(19) United States
(12) Patent Application Publication
(10) Pub. No.: US 2014/0050754 A1
(43) Pub. Date: Feb. 20, 2014

(54) IMMUNOGENIC CHIKUNGUNYA VIRUS PEPTIDES

(71) Applicants: Joo Chuan Tong, Singapore (SG); Jin Kiat Wee, Singapore (SG); Fong Poh Lisa Ng, Singapore (SG); Yiu-Wing Jason Kam, Singapore (SG); Yee Sin Leo, Singapore (SG); Angela Chow, Singapore (SG)

(72) Inventors: Joo Chuan Tong, Singapore (SG); Jin Kiat Wee, Singapore (SG); Fong Poh Lisa Ng, Singapore (SG); Yiu-Wing Jason Kam, Singapore (SG); Yee Sin Leo, Singapore (SG); Angela Chow, Singapore (SG)

(73) Assignees: NATIONAL HEALTHCARE GROUP PTE LTD, Singapore (SG); AGENCY FOR SCIENCE, TECHNOLOGY AND RESEARCH, Singapore (SG)

(21) Appl. No.: 13/992,126

(22) PCT Filed: Dec. 12, 2011
(86) PCT No.: PCT/SG2011/000432

§ 371 Date: Oct. 25, 2013

(30) Foreign Application Priority Data
Dec. 10, 2010 (SG) 201009260-9
Jul. 19, 2011 (SG) 201105239-6

(51) Int. Cl. C07K 14/005; A61K 39/12; G01N 33/569; C07K 16/10

(52) U.S. Cl. CPC C07K 14/005; C07K 16/1081; A61K 39/12; G01N 33/56983
USPC 424/186.1; 530/326; 530/328; 435/188; 530/387.9; 435/325; 435/5

(57) ABSTRACT

The present invention relates to immunogenic peptides of Chikungunya Virus and methods for vaccinating a subject using these peptides. Also disclosed are nucleic acids encoding these peptides and methods for their production.

### CROSS-REFERENCE TO RELATED APPLICATION

This application makes reference to and claims the benefit of priority of an application for “Immunoglobulin (Ig) G binding Chikungunya-associated peptides” filed on Dec. 10, 2010, and an application for “Immunoglobulins (Ig) G-binding Chikungunya peptides” filed on Jul. 19, 2011, with the Intellectual Property Office of Singapore, and there duly assigned applications numbers 201009260-9 and 201105239-6, respectively. The content of said applications respectively filed on Dec. 10, 2010 and Jul. 19, 2011, is incorporated herein by reference for all purposes, including an incorporation of any element or part of the description, claims or drawings not contained herein and referred to in Rule 20.5(a) of the PCT, pursuant to Rule 4.18 of the PCT.

### TECHNICAL FIELD

Various embodiments relate to the field of isolated immunogenic peptides, in particular, isolated immunogenic peptides for treating an Alphavirus infection in a subject.

### BACKGROUND

In several arthralgia causing arbovirus outbreaks, morbidity has been unexpectedly high with extensive incapacitation, including some lethal cases. Some of these arbovirus outbreaks were caused by Chikungunya virus (CHIKV), a virus first isolated in 1953 in Tanzania. Patients infected with CHIKV often developed a contorted posture owing to debilitating joint pains.

The re-emergence of CHIKV since 2005 has caused millions of cases throughout countries around the Indian Ocean and in Southeast Asia. Until now sporadic outbreaks are still ongoing in several countries inflicting naive populations. Singapore, for instance, experienced two successive waves of Chikungunya fever (CHIKF) outbreaks in January and August 2008. Although there were only 718 laboratory-confirmed cases reported in 2008 and 341 cases in 2009, CHIKF remains a public threat due to the low herd immunity. Therefore, the spread of this disease may constitute a major public health problem with severe social and economic impact.

CHIKV is a mosquito-borne virus belonging to the Alphavirus genus of the Togaviridae family. CHIKV is usually transmitted by Aedes mosquitoes.

More specifically, CHIKV is one of the 29 recognised species within the genus Alphavirus in the Togaviridae family (Solignat M, Gay B, Higgs S, Briant L, Devaux C, 2009, “Replication cycle of chikungunya: a re-emerging arbovirus”, Virology 393, pp. 183-197). The virus contains a positive-sense, single-stranded, non-segmented ribonucleic acid (RNA) genome of approximately 11.8 kilobases in length (Strauss J H, Strauss E G, 1994, “The alphaviruses: gene expression, replication, and evolution”, Microbiol Rev 58, pp. 491-562), with a virion diameter of approximately 70 to 100 nm (Simizu B, Yamamoto K, Hashimoto K, Ogata T, 1984, “Structural proteins of Chikungunya virus”, J Virol 51, pp. 254-258). The genome encodes four non-structural proteins (nsP1, nsP2, nsP3 and nsP4) and precursors of structural proteins comprising one capsid protein (C), two envelope surface glycoproteins (E1 and E2) and two additional small proteins (E3 and 6K) (Strauss J H, Strauss E G, supra; Teng T S, Kam Y W, Tan J L, Ng L F P, 2011, “Host responses to Chikungunya virus and perspectives for immune-based therapies”, Future Virology 6, pp. 975-984). Similar to other alphaviruses, the E1 and E2 glycoproteins are postulated to be involved in mediating the fusion and interaction with host receptors during CHIKV infection (Solignat M et al., supra; Voss J E, Vaney M C, Duquerroy S, Vonrhein C, Girard-Blanc C, Crublet E, Thompson A, Bricogne G, Rey F A, 2010, “Glycoprotein organization of Chikungunya virus particles revealed by X-ray crystallography”, Nature 468, pp. 709-712).

CHIKV has a life cycle similar to other alphaviruses and causes sudden onset of fever, rashes, arthritis and other accompanying symptoms. Following the acute phase of the illness, patients develop severe chronic symptoms lasting from several weeks to months, including fatigue, incapacitating joint pain and polyarthritis. However, as in many other arthralgia causing arbovirus infections, the chronic phase is observed only in a fraction of the patients. A role for both innate and adaptive immunity has been proposed but the mechanisms underlying control of viral replication and dissemination, viral clearance, and acute and chronic disease severity remain poorly defined.

The virus is generally maintained in a zoonotic cycle that involves sylvatic and urban CHIKV transmission cycles. Outbreaks occurring in rural countries are mostly due to sylvatic mosquitoes that are capable of infecting both primates and humans, with primates being the primary reservoir for CHIKV. In Asia, CHIKF is identified mostly as an urban disease with humans as the primary reservoir.

Although anti-CHIKV IgM and IgG antibodies have been identified in patients (Panning M, Grywna K, van Esbroeck M, Emmerich P, Drosten C, 2008, “Chikungunya fever in travelers returning to Europe from the Indian Ocean region, 2006”, Emerg Infect Dis 14, pp. 416-422; Yap G, Pok K Y, Lai Y L, Hapuarachchi H C, Chow A, Leo Y S, Tan L K, Ng L C, 2010, “Evaluation of Chikungunya diagnostic assays: differences in sensitivity of serology assays in two independent outbreaks”, PLoS Negl Trop Dis 4: e753), the kinetics of the antibody response have not been well characterized.

Anti-CHIKV IgM and IgG may be detected as early as 10 days from clinical onset, and sero-neutralization assays have confirmed the protective role of anti-CHIKV IgG in infected hosts. However, CHIKV-specific IgG subclass response during clinical progression is unavailable. Understanding the antibody subclass distribution upon CHIKV infection is critical for appropriate prophylactic and therapeutic interventions.

The recognition of CHIKV-associated antigens by the human immune system plays a key role in eliminating CHIKV from the body. This mechanism is based on the prerequisite that there are qualitative or quantitative differences between virus-infected cells and normal human cells. In order to achieve an anti-viral response, the virus-infected cells have to express antigens that are targets of an immune response sufficient for elimination of the virus.

To date, there is no licensed vaccine against CHIKV, although potential CHIKV vaccine candidates have been tested in humans and animals with varying success (Harrison V R, Binn L N, Randall R, 1967, “Comparative immunogenicities of chikungunya vaccines prepared in avian and mammalian tissues”, Am J Trop Med Hyg 16: 786-791; Harrison V R, Eckels K H, Bartelloni P J, Hampton C, 1971, Production and evaluation of a formalin-killed Chikungunya vaccine. J Immunol 107, pp. 643-647; Levitt N H, Ramsburg H H, Hasty S E, Repik P M, Cole F E, Jr., Lupton H W, 1986, “Development of an attenuated strain of chikungunya virus for use in vaccine production”, Vaccine 4, pp. 157-162; Edelman R, Tacket C O, Wasserman S S, Bodison S A, Perry J G, Mangiafico J A, 2000, “Phase II safety and immunogenicity study of live chikungunya virus vaccine TSI-GSD-218”, Am J Trop Med Hyg 62, pp. 681-685; Akahata W, Yang Z Y, Andersen H, Sun S, Holdaway H A, Kong W P, Lewis M G, Higgs S, Rossmann M G, Rao S, et al, 2010, “A virus-like particle vaccine for epidemic Chikungunya virus protects nonhuman primates against infection”, Nat Med 16, pp. 334-338; Plante K, Wang E, Partidos C D, Weger J, Gorchakov R, Tsetsarkin K, Borland E M, Powers A M, Seymour R, Stinchcomb D T, et al, 2011, “Novel chikungunya vaccine candidate with an IRES-based attenuation and host range alteration mechanism”, PLoS Pathog 7, pp. e1002142). Consequently, outbreaks are controlled predominantly by preventing the exposure of people to infected mosquito vectors.

Thus, there is need in the art for vaccines and/or therapeutic antibodies that address the problems mentioned above and exhibit better efficacies and/or lesser drawbacks.

### SUMMARY OF THE INVENTION

In a first aspect, the present invention relates to an isolated immunogenic peptide. The isolated immunogenic peptide is selected from the group consisting of:

(1) peptides comprising the amino acid sequence set forth in any one of SEQ ID Nos. 1 to 95;

(2) peptides consisting of the amino acid sequence set forth in any one of SEQ ID Nos. 1 to 95;

(3) peptides comprising at least 6, 7, 8, 9 or 10 contiguous amino acids of any one of the amino acid sequences set forth in SEQ ID Nos. 96 to 101;

(4) peptides comprising an amino acid sequence that is at least 50, 60, 70, 80 or 90% identical to the sequence of any one of the peptides of (1) to (3);

(5) peptides comprising an amino acid sequence that has at least 50, 60, 70, 80 or 90% sequence similarity to the sequence of any one of the peptides of (1) to (3); and

(6) peptides according to any one of (1) to (5), wherein the peptide comprises at least one chemically modified amino acid.

In a second aspect, a nucleic acid molecule encoding a peptide in accordance with various embodiments of the present invention is provided.

In a third aspect, a vector comprising the nucleic acid molecule in accordance with various embodiments of the present invention is provided.

In a fourth aspect, a recombinant cell comprising the nucleic acid molecule or the vector in accordance with various embodiments of the present invention is provided.

In a fifth aspect, a method for producing a peptide in accordance with various embodiments of the present invention is provided. The method comprises cultivating a recombinant cell in accordance with various embodiments of the present invention in a culture medium under conditions suitable for the expression of the peptide and isolating the expressed peptide from the cultivated cells or the culture medium.

In a sixth aspect, an antibody specifically binding the peptide in accordance with various embodiments of the present invention is provided.

In a seventh aspect, a pharmaceutical composition comprising one or more peptides, one or more nucleic acids, and/or the vector in accordance with various embodiments of the present invention is provided.

In an eighth aspect, a method for vaccinating a subject against Alphaviruses, comprising administering to said subject a therapeutically effective amount of a peptide or a pharmaceutical composition in accordance with various embodiments of the present invention is provided.

In a ninth aspect, a method for treating an Alphavirus infection in a subject, comprising administering to said subject a therapeutically effective amount of a peptide, or a pharmaceutical composition, or an antibody in accordance with various embodiments of the present invention is provided.

In a tenth aspect, a method for monitoring the effectiveness of a treatment of an Alphavirus infection in a subject, comprising contacting a sample obtained from said subject with one or more peptides in accordance with various embodiments of the present invention and determining the level of antibodies specifically binding to said one or more peptides is provided.

In an eleventh aspect, a method for diagnosing an Alphavirus infection in a subject, comprising contacting a sample obtained from said subject with one or more peptides in accordance with various embodiments of the present invention and determining the presence and/or amount of antibodies specifically binding to said one or more peptides in said sample is provided.

In a twelfth aspect, a method for determining the prognosis of a patient infected with Chikungunya-Virus (CHIKV) is provided. The method comprises determining the level of neutralizing IgG3 antibodies specific for a CHIKV antigen in a sample obtained from said patient by contacting said sample with one or more peptides in accordance with various embodiments of the present invention to form peptide:antibody complexes and detecting the presence and amount of said complexes, wherein antibody levels in the post-acute phase that are higher than those of healthy controls are indicative of a lower risk for persistent arthralgia and/or the development of full protective immunity.

In a thirteenth aspect, a method for generating an antibody in accordance with various embodiments of the present invention is provided. The method comprises immunizing a host animal with one or more peptides in accordance with various embodiments of the present invention and (1) isolating the antibodies directed against said one or more peptides from said host animal, or (2) isolating an antibody producing cell that produces antibodies directed against said one or more peptides from said host animal and fusing said antibody producing cell with a myeloma cell to obtain an antibody producing hybridoma cell.

In a fourteenth aspect, the present invention relates to the use of the peptides in accordance with various embodiments of the present invention as a vaccine.

In a fifteenth aspect, the present invention is directed to the use of the peptides in accordance with various embodiments of the present invention as a pharmaceutical agent, such as a therapeutic agent.

In a sixteenth aspect, the invention encompasses also the use of the peptides in accordance with various embodiments of the present invention for the diagnosis of an Alphavirus infection.

### BRIEF DESCRIPTION OF THE DRAWINGS

In the following description, various embodiments of the invention are described with reference to the following drawings, in which:

FIG. 1 shows antibody responses and isotyping of CHIKV-infected patients: (a) virus-specific IgM and IgG antibody titers in plasma samples (n=30), at a dilution of 1:2,000 were determined by ELISA using purified CHIKV virions; (b) virus-specific IgG isotype titers in plasma samples; (c) a profile of IgG3 levels at different time post-illness onset in Early IgG3 and Late IgG3 responders; (d) detection of CHIKV by plasma from CHIKV-infected patients of (i) healthy plasma, (ii) Patient A, and (iii) Patient B; and (e) CHIKV virion-based ELISA being used to determine virus-specific IgG isotype titers in plasma samples (Median 10 days pio, n=30) at a dilution of 1:100, according to various embodiments;

FIG. 2(a) shows images of high throughput immunofluorescence-based cellomics platform of (i) mock; (ii) no plasma; (iii) Early IgG3; (iv) Late IgG3; and (v) healthy plasma, according to various embodiments;

FIG. 2(b) shows in vitro neutralizing activity against CHIKV from plasma samples of Early and Late IgG3 responders for Median 10 days pio, according to various embodiments;

FIG. 3(a) shows plasma samples being added to plates pre-coated with purified CHIK virion for depletion of anti-CHIKV IgG3 Abs, according to various embodiments;

FIG. 3(b) shows depleted samples being subjected to in vitro neutralizing activity detection with a sero-neutralization assay, according to various embodiments;

FIG. 3(c) shows IgG3 antibodies from plasma samples (Median 10 days pio) being depleted and measured for anti-CHIKV IgG3 antibodies with virion-based ELISA, according to various embodiments;

FIG. 3(d) shows depleted samples being subjected to in vitro neutralizing detection in a sero-neutralization assay, according to various embodiments;

FIG. 4 shows (a) viral load in Early IgG3 and Late IgG3 responders during the acute phase of disease; (b) disease severity in Early (High) IgG3 and Late (Low) IgG3 responders during the acute phase of disease; (c) IL-6 levels in Early IgG3 and Late IgG3 responders; (d) comparison of the viral load on median 4 and 10 days pio; (e) persistent arthralgia in Early IgG3 and Late IgG3 responders during the chronic phase of disease, according to various embodiments;

FIG. 5 shows (a) immunoblot analyses for total IgG; (b) anti-CHIKV IgG response for high IgG3; (c) anti-CHIKV IgG response for low IgG3; (d) immunoblot analyses for IgG3; (e) anti-CHIKV IgG3 response for high IgG3; and (f) anti-CHIKV IgG3 response for low IgG3, according to various embodiments;

FIG. 6 shows (a) total cell lysates prepared from transiently expressed capsid protein (Capsid plasmid), E2 glycoprotein (E2 plasmid) and E 1 glycoprotein (E 1 plasmid); (b) total cell lysates prepared from cells transiently transfected with plasmids expressing capsid (Capsid plasmid), E2 (E2 plasmid) and E1 (E1 plasmid); (c) images illustrating purified CHIKV virions subjected to SDS-PAGE and probed with CHIKV-infected patients' plasma at 1:1,000; and (d) outputs from a densitometry reflecting band intensities corresponding to CHIKV structural proteins (Capsid, E2 and E1), according to various embodiments;

FIG. 7 shows measures of absorbance at 450 nm of CHIKV-infected patient plasma (Median 10 days pio) being subjected to peptide-based ELISA using (a) pooled peptides (pool 1-pool 11); and (b) both selected peptide pools (pool 1, pool 2, pool 10 and pool 11) and individual peptides, according to various embodiments;

FIG. 7(c) shows measures of absorbance at 450 nm for selected individual peptides being re-screened with patients' plasma pools, according to various embodiments;

FIG. 8 shows a respective schematic diagram of (a) the localization of the E2 glycoprotein specific epitope (E2EP3); and (b) the localization of E2EP3 in the protein complex situated at the surface of the virus, according to various embodiments;

FIG. 8(c) shows alanine-scan analyses of E2EP3 by anti-CHIKV antibodies, according to various embodiments;

FIG. 8(d) shows alanine substitutions constructed at each position of E2EP3, according to various embodiments;

FIG. 8(e) shows a schematic diagram of the localization of the asparagine (N5) and lysine (K10) residues within the E2EP3 epitope region in the E2 glycoprotein, according to various embodiments;

FIG. 9 shows (a) specific blocking of anti-E2EP3 antibodies in patients' plasma pools; (b) alanine substituted peptides without depletion of E2EP3-specific antibodies in pooled patients' plasma; (c) Anti-CHIKV IgG3 antibodies response from depleted samples using alanine substituted peptides in the depletion assay; and (d) in vitro neutralizing activity of anti-E2EP3 antibodies against CHIKV-infected patients' plasma samples, according to various embodiments;

FIG. 10(a) shows validation of E2EP3 specific IgG3 antibodies in 30 CHIKV-infected patients; according to various embodiments;

FIG. 10 shows (b) CHIK virion-based ELISA used to assess anti-CHIKV IgG titer (whole virus IgG) in CHIKV-infected patients from another Singaporean cohort; (c) screening for IgG3 specific antibodies recognizing E2EP3 in the peptide-based ELISA for CHIKV-infected patients' and healthy donors' plasma of (b); (d) CHIK virion-based ELISA used to assess anti-CHIKV IgG titer (whole virus IgG) in CHIKV-infected patients from another cohort collected in Malaysia; and (e) screening for IgG3 specific antibodies recognizing E2EP3 in the peptide-based ELISA for CHIKV-infected patients' and healthy donors' plasma of (d), according to various embodiments;

FIG. 11 shows (a) E2EP3 specific antibodies titers in non-human primate (NHP) plasma samples; and (b) a graph on percentage infection illustrating specific blocking of anti-E2EP3 antibodies in CHIKV-infected NHP plasma, according to various embodiments;

FIG. 11(c) shows a measure of absorbance at 450 nm for mice within 75 days post infection, according to various embodiments;

FIG. 12 shows a timeline representation of the SGP011 challenge, according to various embodiments;

FIG. 13 shows titer of IgG against KLH-peptides from individual mice with (a) CFA-adjuvanted and (b) PAM3-adjuvanted for Bleed 1; and average titer of IgG against KLH-peptides for (c) CFA-adjuvanted group and (d) PAM3-adjuvanted group for Bleed 1, according to various embodiments;

FIG. 14 shows titer of IgG against KLH-peptides from individual mice with (a) CFA-adjuvanted and (b) PAM3-adjuvanted for Bleed 2; and average titer of IgG against KLH-peptides for (c) CFA-adjuvanted group and (d) PAM3-adjuvanted group for Bleed 2, according to various embodiments;

FIG. 15 shows titer of IgG against SGP11 virion from individual mice with (a) CFA-adjuvanted and (b) PAM3-adjuvanted for Bleed 2; and average titer of IgG against SGP11 virion from individual mice with (c) CFA-adjuvanted and (d) PAM3-adjuvanted for Bleed 2, according to various embodiments;

FIG. 16 shows a graph representing viremia on day 2 post challenge, according to various embodiments;

FIG. 17 shows E2EP3 specific peptide-based ELISA used to measure the titer after E2EP3 peptide vaccination at (a) 19 days post-vaccination, and at (b) 27 days post-vaccination;

FIG. 18 shows (a) in vitro neutralizing activity of E2EP3-vaccinated mouse sera; and (b) output of virus plaque assay (viral load) on mice immunized with E2EP3 or PBS control, according to various embodiments;

FIG. 19 shows CHIKV-induced footpad inflammation: (i) and (iii) represent respective photos of control and infected groups, (ii) and (iv) represent respective photos of control and infected groups, according to various embodiments;

FIG. 20 shows (a) a disease score measurement relative to day 0 for CFA-adjuvant group, (b) footpad sizes relative to day 0 for PAM3-adjuvant group, according to various embodiments;

FIG. 21 shows (a) OD readings of IgG using virion base ELISA, and (b) OD readings of IgM using virion base ELISA, according to various embodiments; and

FIG. 22 shows (a) OD readings of total IgG using E2EP3 peptide-based ELISA, (b) OD readings of IgG3 using E2EP3 peptide-based ELISA (1 in 1000 patients serum dilution), and (c) OD readings of IgG3 using E2EP3 peptide-based ELISA (1 in 200 patients serum dilution), according to various embodiments

FIG. 23 shows a structural analysis of a E2EP3 epitope region, according to various embodiments;

FIG. 24 shows a summary of exemplary algorithms;

FIG. 25 shows single amino acid substitution in peptides (a) 350 and (b) 351 (E2EP3) resulted in alteration of antibody-antigen interactions; (c) a measure of absorbance for (a); and (d) a measure of absorbance for (b), according to various embodiments;

FIG. 26 shows a front view of localisation of peptides 70 to 71, according to various embodiments;

FIG. 27 shows a front view of localisation of peptides 76 to 77, according to various embodiments;

FIG. 28 shows a front view of localisation of peptides (equivalently denoted as SEQ ID Nos.) 41 to 44, according to various embodiments;

FIG. 29 shows a front view of localisation of peptides 62 to 63, according to various embodiments;

FIG. 30 shows a front view of localisation of peptides 64 to 67, according to various embodiments; and

FIG. 31 shows a back view of localisation of peptides 64 to 67, according to various embodiments.

### DETAILED DESCRIPTION

The following detailed description refers to the accompanying drawings that show, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not necessarily mutually exclusive, as some embodiments can be combined with one or more other embodiments to form new embodiments.

In a first aspect, an isolated immunogenic peptide is provided. The isolated immunogenic peptide is selected from the group consisting of: (1) peptides comprising the amino acid sequence set forth in any one of SEQ ID Nos. 1 to 95; (2) peptides consisting of the amino acid sequence set forth in any one of SEQ ID Nos. 1 to 95; (3) peptides comprising at least 6, 7, 8, 9 or 10 contiguous amino acids of any one of the amino acid sequences set forth in SEQ ID Nos. 96 to 101; (4) peptides comprising an amino acid sequence that is at least 50, 60, 70, 80 or 90% identical to the sequence of any one of the peptides of (1) to (3); (5) peptides comprising an amino acid sequence that has at least 50, 60, 70, 80 or 90% sequence similarity to the sequence of any one of the peptides of (1) to (3); or (6) peptides according to any one of (1) to (5), wherein the peptide comprises at least one chemically modified amino acid.

In the context of various embodiments, the term “chemically modified amino acid” may refer to any amino acid that structurally differs from the 20 natural occurring amino acids, namely glycine, alanine, valine, leucin, isoleucin, proline, cysteine, methionine, serine, threonine, glutamine, asparagine, glutamic acid, aspartic acid, lysine, histidine, arginine, phenylalanine, trypthophane, and tyrosine. The term includes amino acids that are chemically modified by adding or deleting a functional group. For example, a chemically modified amino acids comprises any of the natural occurring amino acids that comprises a substitution or modification of one of its functional groups.

As used herein, the term “isolated immunogenic peptide” refers to an immunogenic peptide that has been separated from other peptides or components of a sample or matrix such that it is essentially pure, i.e. free from other contaminating components. For example, an isolated immunogenic peptide may be obtainable by the methods disclosed herein.

In various embodiments, the isolated immunogenic peptide may comprise peptides comprising an amino acid sequence that is about 50%, or about 60%, or about 70%, or about 80% or about 90% identical to the sequence of any one of the peptides (1) comprising the amino acid sequence set forth in any one of SEQ ID Nos. 1 to 95; or (2) consisting of the amino acid sequence set forth in any one of SEQ ID Nos. 1 to 95; or (3) comprising at least 6, 7, 8, 9 or 10 contiguous amino acids of any one of the amino acid sequences set forth in SEQ ID Nos. 96 to 101.

In other embodiments, the isolated immunogenic peptide may comprise peptides comprising an amino acid sequence that has about 50%, or about 60%, or about 70%, or about 80% or about 90% sequence similarity to the sequence of any one of the peptides (1) comprising the amino acid sequence set forth in any one of SEQ ID Nos. 1 to 95; or (2) consisting of the amino acid sequence set forth in any one of SEQ ID Nos. 1 to 95; or (3) comprising at least 6, 7, 8, 9 or 10 contiguous amino acids of any one of the amino acid sequences set forth in SEQ ID Nos. 96 to 101.

As used herein, the term “sequence identity” in relation to a peptide sequence, refers to the degree of amino acid sequence identity between 2 peptide sequences. By way of example only, a sequence identity of 50% between two peptides of 10 amino acids length thus means that 5 of the amino acids are identical whereas the other 5 are different. The term “sequence similarity”, as used herein in relation to a peptide, refers to the degree of amino acid similarity between 2 different peptides. “Similarity” in this context refers to amino acids that have similar properties, i.e. so-called conservative amino acid substitutions. Examples for such conservative amino acid substitutions are substitutions that occur within one group of amino acids with similar properties. These groups include aromatic amino acids (Phe, Tyr and Trp), polar amino acids (Ser, Thr, Gln, Asn, Cys), basic amino acids (Lys, Arg, His), acidic amino acids (Glu and Asp) and non-polar amino acids (Gly, Ala, Val, Leu, Ile, Met).

As used herein, a “peptide” generally has from about 3 to about 100 amino acids, whereas a polypeptide or protein has about 100 or more amino acids, up to a full length sequence translated from a gene. Additionally, as used herein, a peptide can be a subsequence or a portion of a polypeptide or protein. In certain embodiments the peptide consists of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acid residues.

As used herein, an “amino acid residue” refers to any naturally or non-naturally occurring amino acid, any amino acid derivative or any amino acid mimic known in the art. Included are the L− as well as the D− forms of the respective amino acids, although the L forms are usually preferred. In various embodiments, the term relates to the 20 naturally occurring amino acids glycine, alanine, valine, leucin, isoleucin, proline, cysteine, methionine, serine, threonine, glutamine, asparagine, glutamic acid, aspartic acid, lysine, histidine, arginine, phenylalanine, trypthophane, and tyrosine in their L form.

In various embodiments, the peptide may be 10 to 50 amino acids in length. In other embodiments, the peptide may be 15 to 25 amino acids in length.

For example the peptide may comprise a B-cell epitope. A B-cell epitope refers to a peptide sequence that is recognized and bound by a B cell receptor with detectable affinity.

As used herein, the term “bind” may generally refer to combine chemically or form a chemical bond. The term “detectable affinity” may refer to a level of binding strength of the peptide and the receptor, or an antibody to an antigen that can be quantified and/or measurable by standard techniques. For example, detectable affinity may be determined by a binding assay. A detectable affinity range may be observable by, for example but not limited to, surface plasma resonance (SPR) detection.

For example, various embodiments of the present invention may relate to CHIKV-associated peptides that are capable of binding to a molecule of the immunoglobulin (Ig) class of molecules. Such peptides may be, for example, used to design therapeutic and prophylactic agents (i.e. drugs, vaccines) against alpha-viruses such as CHIKV.

Particularly, the inventors have found that the immunoglobulin (Ig) G3 subclass may play a critical role in the clearance of viruses from the human body. In order to elicit an IgG3 immune reaction, foreign proteins/peptides have to be presented to the B cells. B cells recognize antigens as (i) linear, contiguous stretches of amino acids within a protein, or (ii) discontinuous (or non-linear) stretches of amino acids that are brought together spatially by protein folding. It has been estimated that ˜10% of all B cell epitopes are contiguous in nature, with the remainder being discontinuous in structure. In order for an antigen to elicit a humoral immune response, it needs to bind to a B cell receptor. This process may depend on the specificity of the B-cell receptor and on the amino acid sequence of the peptide. In general, B-cell epitopes have a length that varies from 5 to 20 amino acids.

A critical component in the design and development of an anti-viral vaccine is the identification and characterization of viral-associated antigens being recognized by IgG.

The CHIKV antigens, or their epitopes, that are recognized by IgG3 may be molecules derived from the viral proteins. The presence of epitopes in the amino acid sequence of the antigen is absolutely mandatory since only such peptides lead to a B cell response, either in vitro or in vivo.

Therefore, viral-derived peptides may be a starting point for the development of a vaccine against a virus. The methods for identifying and characterizing the peptide sequences may be based on the use of IgG antibodies that have already been induced in the patients.

Because only the antigen epitopes—not the entire antigen—elicit a B cell response, it is therefore important to select only those peptides that are recognized by B cell receptors, so that targets for the specific recognition of viral cells by appropriate B cell receptors are obtained.

For example, peptides may be used for stimulating an immune response that comprise SEQ ID Nos. 1 to 95, and in which at least one amino acid is optionally replaced by another amino acid with similar chemical properties.

Amino acids within the antibody binding site may be replaced by amino acids with similar chemical properties while still retaining the predominant binding of a certain IgG subtype. Thus, for example, in peptides associated with the IgG3 subtype, leucine on position 5 may be replaced by isoleucine, valine or methionine and vice versa, and at the position 8 leucine by valine, isoleucine or alanine, each containing non-polar side chains, without significantly affecting binding affinity.

Furthermore peptides with SEQ ID Nos. 1 to 95 comprising at least one additional amino acid N- or/and C-terminally, or in which at least one amino acid is deleted, may be used.

Furthermore, peptides with SEQ ID Nos. 1 to 95 in which at least one amino acid is chemically modified may be used. The modified amino acid(s) is (are) selected in such way that the modification does not affect the immunogenicity of the peptide, i.e. the peptide demonstrates a similar binding affinity to the IgG molecule and the capability for B cell stimulation.

In various embodiments, the dissociation constant K_{D} of the peptide for the B cell receptor may be at least about 10⁻⁶ M. For example, the K_{D} of the peptide for the B cell receptor may be about 10⁻⁷ M, or about 10⁻⁸ M or even lower. The peptide may be capable of eliciting an IgG or IgM antibody response in a human subject.

In the context of various embodiments, the term “antibody response” generally refers to the generation of antibodies against a given antigen. Factors determining whether an antigen stimulates an antibody response may include a degree of foreignness, size and complexity, dosage of antigen administered, and genetic makeup of host. For example, an antibody response may be a rapid production of antibodies in response to an antigen in an individual who was exposed previously to the same antigen. In one embodiment, the antibody response may be an IgG3 antibody response.

In various embodiments, the peptide may be coupled to a detectable label.

As used herein, the term “detectable” may refer to capable of being ascertained of presence, using various techniques such as fluorescence detection. For example, the label may be selected from the group consisting of a fluorophor, a chromophor, a radiolabel, biotin, streptavidin, a Strep-tag, a 6×His-tag, a Myc-tag, and an enzyme.

In a second aspect, a nucleic acid molecule encoding a peptide in accordance to various embodiments is provided.

The term “nucleic acid molecule” as used herein refers to any nucleic acid in any possible configuration, such as single stranded, double stranded or a combination thereof. Nucleic acids include for instance DNA molecules (e.g., cDNA or genomic DNA), RNA molecules (e.g., mRNA), analogues of the DNA or RNA generated using nucleotide analogues or using nucleic acid chemistry, and PNA (protein nucleic acids). DNA or RNA may be of genomic or synthetic origin and may be single or double stranded. A nucleic acid molecule may furthermore contain non-natural nucleotide analogues and/or be linked to an affinity tag or a label.

Also encompassed by the present invention are nucleic acid sequences substantially complementary to the above nucleic acid sequence. “Substantially complementary” as used herein refers to the fact that a given nucleic acid sequence is at least 90, for instance at least 95, and in some embodiments 100% complementary to another nucleic acid sequence. The term “complementary” or “complement” refers to two nucleotides that can form multiple favourable interactions with one another. Such favourable interactions include and preferably are exclusively Watson-Crick base pairing. A nucleotide sequence is the full complement of another nucleotide sequence if all of the nucleotides of the first sequence are complementary to all of the nucleotides of, the second sequence

For example, the nucleic acid molecule may be a DNA or RNA molecule, and may also be used for immune therapy of an Alphavirus infection, for example but not limited to, CHIKV infection. The peptide which is expressed from the nucleic acid molecule may induce an immune response against CHIKV cells expressing the peptide.

According to a third aspect, the present invention relates to a vector comprising the nucleic acid molecule. The vector may be a plasmid.

The term “vector” relates to a single or double-stranded circular nucleic acid molecule that can be introduced, e.g. transfected, into cells and replicated within or independently of a cell genome. A circular double-stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule encoding an allergen or a fragment thereof can be inserted into a vector by cutting the vector with restriction enzymes and ligating the two pieces together.

In a fourth aspect, a recombinant cell comprising the nucleic acid molecule or the vector is provided.

The term “recombinant cell” may refer to a biological cell that is produced genetic engineering and includes cells that have been genetically engineered such that they contain a nucleic acid sequence that has been artificially introduced into such cells and comprises at least partially non-native sequences.

In various embodiments, the cell may be a prokaryotic cell. In other embodiments, the cell may be a eukaryotic cell.

In an example, cells may be genetically altered using a nucleic acid molecule encoding one or more of the peptides comprising or having the amino acid set forth in any one of SEQ ID NOs. 1 to 95.

For this purpose, the cells may be transfected with the respective DNA sequence encoding the peptides.

In a fifth aspect, a method for producing a peptide in accordance to various embodiments is provided. The method comprises cultivating a recombinant cell in accordance to various embodiments in a culture medium under conditions suitable for the expression of the peptide and isolating the expressed peptide from the cultivated cells or the culture medium. The method may be an in vitro (ex vivo) method or an in vivo method.

In this context, the term “suitable” with respect to the term “conditions” may generally refer to any requirements or settings that allow the expression of the peptide to occur and/or the expressed peptide to be isolated from the cultivated cells or the culture medium. For example, a suitable condition may be of a particular temperature or pressure, or may involve a particular additive or a specific amount thereof.

In a sixth aspect, an antibody specifically binding the peptide in accordance to various embodiments is provided.

The antibody may bind the peptide with a dissociation constant (K_{D}) of at least 10⁻⁶ M. For example, the K_{D} of the peptide may be about 10⁻⁷ M, or about 10⁻⁸ M or even lower.

In a seventh aspect, the invention relates to a pharmaceutical composition comprising one or more peptides, or one or more nucleic acids, or the vector in accordance to various embodiments. The pharmaceutical composition may be a combination of any of the one or more peptides, the one or more nucleic acids, and the vector in accordance to various embodiments.

The term “pharmaceutical composition” may refer to a vaccine composition comprising one or more one or more peptides, or one or more nucleic acids, or the vector in accordance to various embodiments of the invention. Such a vaccine composition is usually administered, e.g. injected, once or multiple times to a subject in order to elicit a protective immune response against Alphaviruses, including, but not limited to, CHIKV. The “pharmaceutical composition” may also refer to a diagnostic composition comprising one or more peptides of the invention for diagnosing Alphavirus infection, including, but not limited to, CHIKV infection in a subject. In still further embodiments, the “pharmaceutical composition” may be a therapeutic composition comprising one or more peptides, or one or more nucleic acids, or the vector in accordance to various embodiments of the invention for treating Alphavirus infection including, but not limited to, CHIKV infection in a subject.

In various embodiments, the pharmaceutical composition may further comprise a pharmaceutically acceptable carrier and/or pharmaceutically acceptable excipients.

The pharmaceutical composition may be used, for example, for parenteral administration, such as subcutaneous, intradermal or intramuscular, or for oral application. For this, the peptides may be solved or suspended in a pharmaceutically acceptable, preferably aqueous carrier. Furthermore, the composition may contain excipients such as buffers, binders, and diluents.

The pharmaceutical composition may further comprise at least one immunostimulatory agent. The at least one immunostimulatory agent may be selected from the group consisting of adjuvants and cytokines. For example, the at least one immunostimulatory agent may be at least one adjuvant selected from the group consisting of complete and incomplete Freud's adjuvant, tripalmitoyl-S-glyceryl-cystein, aluminium salts, virosomes, squalene, MF59, monophosphoryl lipid A, QS21, CpG motifs, ISCOMS (structured complex of saponins and lipids), and Advax.

In another example, the peptides may also be given together with immunostimulatory substances such as cytokines. A comprehensive description of excipients that may be used in such compositions is given, for example in A. Kibbe, Handbook of Pharmaceutical Excipients, 3. Ed., 2000, American Pharmaceutical Association and pharmaceutical press.

In various embodiments, the pharmaceutical composition may comprise a peptide in accordance to various embodiments bound to an antigen-presenting cell (APC).

In an eighth aspect, a method for vaccinating a subject against Alphaviruses, comprising administering to said subject a therapeutically effective amount of a peptide or a pharmaceutical composition in accordance to various embodiments is provided. In various embodiments, said administering step may be repeated at least once. As used herein, the subject may be a mammal, preferably a human.

In a ninth aspect, a method for treating an Alphavirus infection in a subject, comprising administering to said subject a therapeutically effective amount of a peptide, or a pharmaceutical composition, or an antibody in accordance to various embodiments is provided. The method may be an in vitro (ex vivo) method or an in vivo method.

For example, the peptide may be used for treatment and prophylaxis of CHIKV infection and/or alphavirus infection.

Independent studies have shown that the peptides according to various embodiments of the invention are suitable for such use. In these studies it has been shown that specifically generated IgG that are specific for certain peptides were able to neutralize CHIKV effectively and selectively.

Basically, for the use of viral-associated antigens in a viral vaccine, several application forms were possible. For example, the antigen may be administered either as recombinant protein together with suitable adjuvants or carrier systems, or in form of the cDNA encoding the antigen in plasmid vectors.

For example, the pharmaceutical composition may be used for prevention, prophylaxis and/or therapy of CHIKV infection and/or alphaviral infections in general.

The pharmaceutical composition containing at least one of the peptides with SEQ ID NOs. 1 to 95 may be administered to a patient suffering from a CHIKV infection with which the respective peptide or antigen is associated. Thus, a CHIKV-specific immune response based on viral-specific IgG may be elicited.

The amount of the peptide or peptides in the pharmaceutical composition is present in a therapeutically effective amount. The peptides that are present in the composition may also bind to at least two different immunoglobulins.

In a tenth aspect, a method for monitoring the effectiveness of a treatment of an Alphavirus infection in a subject is provided. The method comprises contacting a sample obtained from said subject with one or more peptides in accordance to various embodiments and determining the level of antibodies specifically binding to said one or more peptides. The method may be an in vitro (ex vivo) method or an in vivo method. For example, the sample may be mixed with the one or more peptides and the level of antibodies specifically binding to said one or more peptides may be measured or observed using a binding assay.

In an eleventh aspect, a method for diagnosing an Alphavirus infection in a subject, comprising contacting a sample obtained from said subject with one or more peptides in accordance to various embodiments and determining the presence and/or amount of antibodies specifically binding to said one or more peptides in said sample. The method may be an in vitro (ex vivo) method or an in vivo method. In various embodiments, the sample may be a body fluid, or a cell or a tissue sample.

In one embodiment, the sample may be a body fluid sample and the body fluid may be selected from the group consisting of blood, serum, plasma, urine, synovial fluid, lymph, saliva, tears, liquor cerebrospinalis, vaginal fluid, and semen.

In various embodiments, the Alphavirus may be selected from the group consisting of Chikungunya Virus (CHIKV), Sindbis Virus, Semliki Forest Virus, Mayaro Virus, Ross River Virus, Barmah Forest Virus, Eastern Equine Encephalitis Virus, Western Equine Encephalitis Virus, O'Nyong Nyong Virus (ONNV), Venezuelan Equine Encephalitis Virus, Aura Virus, Bebaru Virus, Cabassou Virus, Eastern Everglades Virus, Fort Morgan Virus, Getah Virus, Highlands J Virus, Middelburg Virus; Mosso das Pedras Virus (78V3531), Mucambo Virus, Ndumu Virus, Pixuna Virus, Rio Negro Virus, Salmon Pancreas Disease Virus, Southern Elephant Seal Virus, Tonate Virus, Trocara Virus, Una Virus, and Whataroa Virus. For example, the Alphavirus may be Chikungunya Virus (CHIKV).

In a twelfth aspect, a method for determining the prognosis of a patient infected with Chikungunya-Virus (CHIKV) is provided. The method comprises determining the level of neutralizing IgG3 antibodies specific for a CHIKV antigen in a sample obtained from said patient by contacting said sample with one or more peptides in accordance to various embodiments to form peptide:antibody complexes and detecting the presence and amount of said complexes, wherein antibody levels in the post-acute phase that are higher than those of healthy controls are indicative of a lower risk for persistent arthralgia and/or the development of full protective immunity. The method may be an in vitro (ex vivo) method or an in vivo method.

In various embodiments, the antibody levels in the post-acute phase that are higher than the mean value obtained from healthy controls±3SD (standard deviation) may be indicative of a lower risk for persistent arthralgia and/or the development of full protective immunity. The CHIKV antigen may be a CHIKV E2 glycoprotein antigen.

In a thirteenth aspect, a method for generating an antibody in accordance to various embodiments is provided. The method comprises immunizing a host animal with one or more peptides in accordance to various embodiments and (1) isolating the antibodies directed against said one or more peptides from said host animal, or (2) isolating an antibody producing cell that produces antibodies directed against said one or more peptides from said host animal and fusing said antibody producing cell with a myeloma cell to obtain an antibody producing hybridoma cell. The method may be an in vitro (ex vivo) method or an in vivo method.

For example, the peptides may be used to generate an antibody. Polyclonal antibodies may be obtained conventionally by immunizing animals by injection of the peptides and subsequent purification of the immunoglobulin.

Monoclonal antibodies may be generated according to standard protocols, such as, for example, described in Methods Enzymol. (1986), 121, Hybridoma technology and monoclonal antibodies.

In a fourteenth aspect, a use of the peptides in accordance to various embodiments as a vaccine is provided.

Synthetic peptides may be used as a vaccine. For this purpose, the peptide may be used in an embodiment together with added adjuvants, or alone. As an adjuvant, for example, the granulocyte macrophage colony stimulating factor (GMCSF) may be used. Further examples for such adjuvants are aluminum hydroxide, mineral oil emulsions such as, for example, Freund's adjuvant, saponins or silicon compounds. The use of adjuvants provides the advantage that the immune response induced by the peptide may be enhanced, and/or the peptide may be stabilized.

The antigen presenting cells carrying the peptide may be used either directly or may be activated prior to their use, for example with the heat shock protein gp96. This heat shock protein induces the expression of MHC class I molecules and co-stimulatory molecules such as B7, and also stimulates the production of cytokines. Together, this supports the induction of immune responses.

In a fifteenth aspect, a use of the peptides in accordance to various embodiments as a pharmaceutical agent is provided.

In a sixteenth aspect, a use of the peptides in accordance to various embodiments for the diagnosis of an Alphavirus infection is provided.

For example, the peptide may be used as a marker to evaluate the progress of a therapy for a viral infection.

The peptide may be used in other immunizations or therapies for monitoring the therapy as well. Therefore, the peptide may not only be used therapeutically but also diagnostically.

In the context of various embodiments, the term “about” or “approximately” as applied to a numeric value encompasses the exact value and a variance of +/−5% of the value.

The phrase “at least substantially” may include “exactly” and a variance of +/−5% thereof. As an example and not limitation, the phrase “A is at least substantially the same as B” may encompass embodiments where A is exactly the same as B, or where A may be within a variance of +/−5%, for example of a value, of B, or vice versa.

In the context of the present invention, the term “comprising” means including, but not limited to, whatever follows the word “comprising”. Thus, use of the term “comprising” indicates that the listed elements are required or mandatory, but that other elements are optional and may or may not be present. The term “consisting of” means including, and limited to, whatever follows the phrase “consisting of”. Thus, the phrase “consisting of” indicates that the listed elements are required or mandatory, and that no other elements may be present.

Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to the skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject-matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

In order that the invention may be readily understood and put into practical effect, particular embodiments will now be described by way of examples and not limitations, and with reference to the figures.

### EXAMPLES

It is to be understood by those skilled in the art that the identified CHIKV peptides may be synthesized to obtain larger quantities or for the use for the below described purposes, or may be expressed in cells.

The above mentioned peptides from CHIKV were isolated and identified as specific ligands from IgG molecules. The term “CHIKV-associated” peptides herein refer to peptides that are isolated and identified from CHIKV material.

The specific ligands may be used in immunotherapy, e.g. to induce an immune response against CHIIKV expressing the respective antigens from which the peptides are derived.

Such an immune response in form of an induction of Cytotoxic T-Lymphocyte (CTL) may be obtained in vivo. In order to obtain such an immune response the peptide is administered to a patient suffering from a CHIKV infection, for example in form of a pharmaceutical composition.

On the other hand, a CTL response against CHIKV expressing the antigens from which the peptides are derived may also be elicited ex vivo. In order to do so, the IgG precursor cells were incubated together with antigen presenting cells and the peptides. Then, the thus stimulated CTL were cultivated, and these activated CTL were administered to the patient.

Furthermore, antigen-presenting cells (APC) were loaded with the peptides ex vivo, and to administer these loaded APC to the CHIKV patient the antigens from which the peptide is derived. Then, the APC themselves may present the peptide to the IgG in vivo, and thereby activate them.

However, the peptides according to various embodiments of the invention may also be used as diagnostic reagents.

Thus, using the peptides it may be found out if IgG are present in an IgG population or have been induced by a therapy that are specifically directed against a peptide.

The peptides may also be used to test for the increase of precursor IgGs with reactivity against the defined peptide.

Furthermore, the peptide may be used as a marker to track the disease course of a viral infection expressing the antigen from which the peptide is derived.

SEQ ID Nos 1 to 95 contain proteins from which the peptides are derived, and the respective positions of the peptides in the respective proteins. The Acc numbers are listed that are used in the gene bank of the “National Center for Biotechnology Information” of the National Institute of Health (see http://www.ncbi.nlm.nih.gov/).

The following examples are provided to further illustrate the present invention and are not intended to be limiting to the scope of the invention.

### Materials and Methods

Patients and Plasma Collection.

Thirty patients, who were admitted with acute CHIKF to the Communicable Disease Centre at Tan Tock Seng Hospital (CDC/TTSH) during the outbreak from Aug. 1 to Sep. 23, 2008 were included in this study. Written informed consent was obtained from all participants. This study was approved by the National Healthcare Group's Domain-specific Ethics Review Board (DSRB Reference No. B/08/026). Plasma specimens were collected at 4 time points post-illness onset (pio): (1) at acute phase (median 4 days pio); (2) at early convalescent phase (median 10 days pio); at late convalescent phased (4-6 weeks pio); at chronic phase (2-3 months pio).

Clinical features definition and clinical samples were as described in Win M K, Chow A, Dimatatac F, Go C J, Leo Y S., “Chikungunya fever in Singapore: acute clinical and laboratory features, and factors associated with persistent arthralgia”, J Clin Virol, 2010, 49, pp. 111-114, and Ng K W, Chow A, Win M K, et al., “Clinical features and epidemiology of chikungunya infection in Singapore”, Singapore Med J, 2009, 50, pp. 785-790.

Illness was defined as “severe”, if a patient had either a maximum temperature greater than 38.5° C., or a maximum pulse rate greater than 100 beats/minute, or a nadir platelet count less than 100×10⁹/L. Arthralgia was defined as having pain in one or more joints, with or without joint inflammation. Patients were later clustered into early IgG3 and late IgG3 responders based on their IgG3 titer measured on median 10 days pio (Table 1).

TABLE 1
Demographic Characteristics, Immunological and Disease Profiles

| | | | Anti- | Anti-CHIKV | |
|---|---|---|---|---|---|
| Patient (Sex, | Duration of fever, | Acute illness | CHIKV IgG | IgG3 | |
| Age in years) | Days | severity^{a} | titer^{b} | classification^{c} | Clinical outcome^{d} |
| CHIKV 1 (M, 40) | 3 | Severe | Low | Early | Complete recovery |
| CHIKV 2 (M, 23) | 8 | Severe | High | Early | Complete recovery |
| CHIKV 3 (M, 62) | 7 | Severe | High | Early | Lethargy, weakness |
| CHIKV 4 (M, 43) | 5 | Severe | Low | Early | Complete recovery |
| CHIKV 5 (M, 29) | 6 | Severe | Low | Early | Complete recovery |
| CHIKV 6 (M, 35) | 7 | Severe | High | Early | Complete recovery |
| CHIKV 7 (M, 30) | 4 | Severe | Low | Early | Complete recovery |
| CHIKV 8 (M, 35) | 4 | Severe | High | Early | Complete recovery |
| CHIKV 9 (M, 26) | 3 | Severe | High | Early | Complete recovery |
| CHIKV 10 (M, 28) | 4 | Severe | Low | Early | Complete recovery |
| CHIKV 11 (M, 49) | 2 | Severe | Low | Early | Complete recovery |
| CHIKV 12 (M, 50) | 6 | Severe | Low | Early | Complete recovery |
| CHIKV 13 (M, 38) | 3 | Severe | High | Early | Complete recovery |
| CHIKV 14 (M, 60) | 3 | Mild | Low | Early | Complete recovery |
| CHIKV 15 (F, 62) | 7 | Severe | High | Early | Complete recovery |
| CHIKV 16 (M, 45) | 0 | Mild | High | Early | Complete recovery |
| CHIKV 17 (M, 34) | 3 | Mild | High | Late | Complete recovery |
| CHIKV 18 (M, 29) | 2 | Severe | Low | Late | Persistent arthralgia |
| CHIKV 19 (F, 67) | 7 | Mild | High | Late | Complete recovery |
| CHIKV 20 (M, 24) | 3 | Mild | Low | Late | Complete recovery |
| CHIKV 21 (M, 34) | 0 | Mild | High | Late | Complete recovery |
| CHIKV 22 (M, 28) | 7 | Mild | High | Late | Complete recovery |
| CHIKV 23 (M, 42) | 2 | Mild | Low | Late | Complete recovery |
| CHIKV 24 (F, 40) | 6 | Mild | Low | Late | Persistent arthralgia |
| CHIKV 25 (F, 31) | 6 | Mild | Low | Late | Persistent arthralgia |
| CHIKV 26 (M, 46) | 9 | Mild | High | Late | Complete recovery |
| CHIKV 27 (M, 26) | 4 | Mild | Low | Late | Persistent arthralgia |
| CHIKV 28 (M, 28) | 5 | Severe | Low | Late | Complete recovery |
| CHIKV 29 (M, 47) | 8 | Mild | Low | Late | Complete recovery |
| CHIKV 30 (M, 39) | 1 | Mild | High | Late | Complete recovery |

^{a}Severity was defined as having a temperature >38.5° C., pulse rate >100 beats/min, or platelet count <100 × 10⁹ cells/L.
^{b}Anti-CHIKV IgG antibody titer was determined by virion-based ELISA from plasma samples collected at 7-10 days post-illness onset. O.D. values > median value of 0.46 were classified as “High” and the rest were defined as “Low”.
^{c}Anti-CHIKV IgG3 isotype titer and response was determined by virion-based ELISA from plasma collected at 7-10 days post-illness onset. During this phase, approximately half of the patient group has already a significant increase of IgG3 antibodies, segregating this cohort into “Early” and “Late” IgG3 responders.
^{d}Clinical outcome at chronic phase that is 2-3 months after post-illness onset.

Computational Mapping.

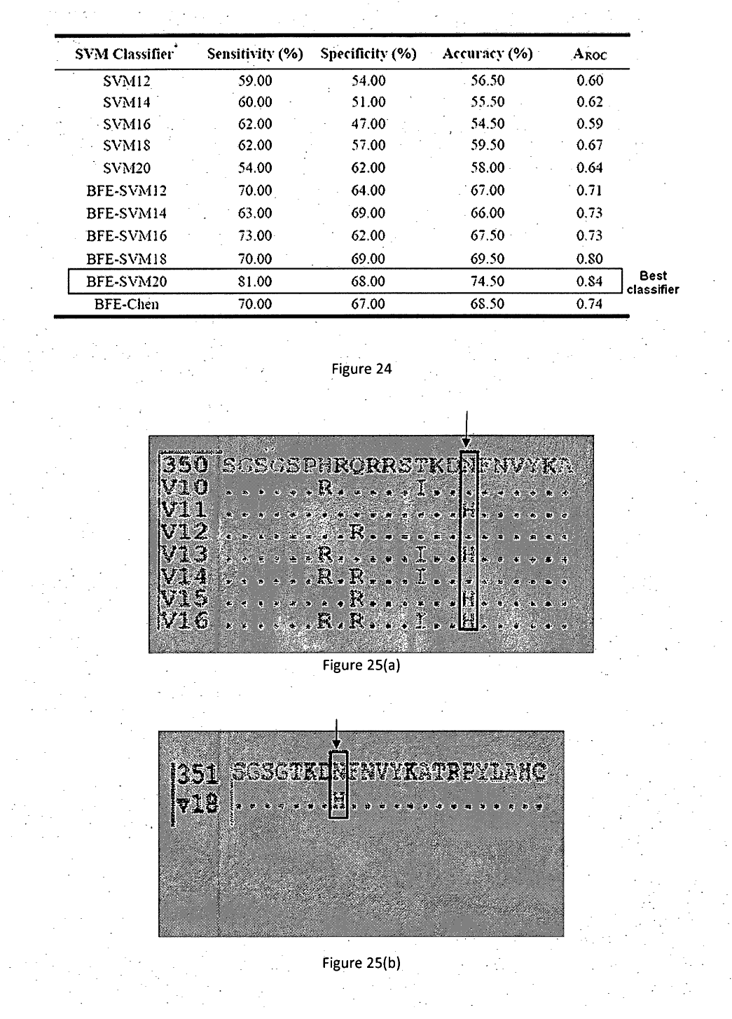

Computational mapping of B-cell epitope sequences on CHIKV proteins was performed using the BayesB web-server available at http://www.immunopred.org/bayesb/index.html. The system achieved an accuracy of about 74.50% and A_{ROC} of about 0.84 on an independent test set and was shown to outperform existing linear B-cell epitope prediction algorithms (FIG. 24 summarizing exemplary algorithms). The best classifier from BayesB outperformed other existing methods. Accuracy and Aroc generally improved over longer peptide lengths.

In comparison to computational mapping, “Wet-lab” examples can be expensive and time-consuming.

Computational prediction may advantageously generate high-throughput experimental leads for further validation; thereby being much cheaper and faster.

Computational prediction may also complement discovery of novel structural features involved in B-cell linear epitope binding.

Plasmid DNA Transfection and Virus Infection (Transient Transfection).

Recombinant CHIKV structural proteins were expressed in HEK 293T cells as described in Song W, Lahiri D K, “Efficient transfection of DNA by mixing cells in suspension with calcium phosphate”, Nucleic Acids Res., 1995, 23, pp. 3609-3611 with modifications. Cells were transfected (20 μg of plasmid DNA per 5×10⁶ cells) using CaPO₄. At about 24 hours post-transfection, cells were washed with PBS and lysed with ice-cold lysis buffer (20 mM Hepes, pH 7.5, 280 mM KCl, 1 mM EDTA, 10% glycerol, 1% NP-40) containing protease inhibitors (20 mM NaF, 0.1 mM Na₃VO₃, 1 mM DTT, 1 mM PMSF). Cell lysates were mixed with Laemmli buffer and stored at about −20° C. for Western blot analyses.

Western Blots.

Cell lysates were collected from 293T producing cells using ice-cold lysis buffer. 50 μg of whole-cell lysate were loaded on 10% SDS-PAGE and transferred onto nitrocellulose membrane at 144 V for about 45 min. Protein immunoblotting analysis was done with plasma samples (Singapore, TTSH) diluted in a ratio of 1:2000, and a secondary antibody (goat anti-human IgG peroxidase conjugate) in a dilution of 1:10000. Bands were visualized on X-ray films (Kodak) by chemiluminescence (Amersham Biosciences).

Virus Production and Purification for Virion-Based ELISA.

The Singapore strain (SGP11) was isolated from a CHIKF patient (Her Z, Malleret B, Chan M, Ong E K, Wong S C, Kwek D J, Tolou H, Lin R T, Tambyah P A, Renia L, et al., “Active infection of human blood monocytes by Chikungunya virus triggers an innate immune response”, J Immunol., 2010, 184, pp. 5903-5913). Virus was propagated in VeroE6 cells and viral particles were purified by ultra-centrifugation as follows: infected culture medium was filtered with 0.45 μm filters after cell debris was removed by centrifugation at 2,000 rpm for about 5 minutes at about 4° C. Clear supernatant was centrifuged at 28,000 rpm for about 3 hours at about 4° C., in the presence of a 20% sucrose cushion. Supernatant was removed and virus particles were reconstituted with 100 μl of Tris/EDTA (TE) buffer and stored in aliquots at about −80° C. Purified CHIK virions were quantified by quantitative reverse transcriptase-PCR (qRT-PCR).

Virion-Based ELISA and Isotyping of CHIKV-Infected Patient Samples.

Polystyrene 96-well microtiter plates (MaxiSorp, Nunc) were coated with purified Chikungunya virus (20000 virion/μl in PBS; 50 μl/well). Wells were blocked with PBST-milk (PBS, 0.05% Tween 20, 5% non-fat milk) and plates were incubated for about 1.5 hours at about 37° C. Plasma samples were then diluted 1:500, 1:2000 in PBST-milk and incubated 1 hour at about 37° C. HRP-conjugated mouse anti-human IgG, IgG1, IgG2, IgG3, IgG4 and IgM (Molecular Probes) were used to detect human antibodies bound to virus-coated wells. Reactions were developed using TMB substrate (Sigma-Aldrich) and stopped with stopping reagent (Sigma-Aldrich). The absorbance was measured at 450 nm. Healthy donor samples were used as controls. ELISA determinations were done in duplicates and the values plotted as means±standard error means (SEM).

Antigenic responses were detected by immunofluorescence assay as described. HEK 293T cells were seeded on coverslips coated with human plasma fibronectin (Sigma-Aldrich). Virus infection was performed at multiplicity of infection (MOI) of 10. At about 6 hours post-infection, cells were fixed with PBS containing 4% paraformaldehyde. Cells were then permeabilized in PBS containing 0.2% Triton-X and blocked with PBS supplied with 10% FBS. Cells were stained with patients' plasma diluted in PBS (1:500) containing 1% BSA for about 1 hour at about 37° C. This was followed by incubation with goat anti-human secondary antibody conjugated to Alexa Fluor 488 (Molecular Probes) for about 1 hour at about 37° C. Cells were washed, mounted and examined with confocal laser-scanning microscope (Fluoview FV 100; Olympus) using 20×NA 0.75 or 60×NA 1.42 objective. Images were collected using FV10-ASW software and processed with Adobe Photoshop software. Levels of cytokines were measured by multiplex bead-based arrays as described in Ng L F, Chow A, Sun Y J, et al., “IL-1beta, IL-6, and RANTES as biomarkers of Chikungunya severity”, PLoS One, 2009, 4, e4261.

Sero-Neutralization Assay.

Neutralizing activity of CHIKV-infected patient samples were test in triplicates and were analyzed by immunofluorescence-based cell infection assay in HEK 293T cells, using Singapore strain CHIKV (SGP11). CHIKV were mixed at MOI 10 with diluted (1:100, 500 or 1,000) heat-inactivated human plasma and incubated for 2 hours at about 37° C. with gentle agitation (350 rpm). Virus-antibody mixtures were then added to HEK 293T cells seeded in 96-well plates and incubated for 1.5 hours at about 37° C. Virus inoculums (medium) were removed, and cells were replenished with DMEM medium supplied with 5% FBS and incubated for about 6 hours at about 37° C. before fixation with 4% paraformaldehyde followed by immunofluorescence quantification using the Cellomics ArrayScan V. The Cellomics ArrayScan. V was used as a complementary means of assessing neutralization capabilities of patients' plasma (same set-up as mentioned above), but involves an assessment endpoint at 6 hours post-infection (pi) to capture possible early protective responses against virus infection. Percentage infectivity was detected with High Content Screening and was calculated according to the equation: % Infectivity=100×(% responder from sero-neutralization group/% responder from virus infection group).

Epitope Determination and Structural Localization.

Peptide-based ELISA was performed to screen CHIKV-infected patients' plasma for viral epitopes using synthesized biotinylated-peptides (Mimotopes). Eighteen-mer overlapping peptides were generated from consensus sequence based on alignments of different CHIKV amino acid sequences (accession numbers: EF452493, EF027139, DQ443544, EU703760, EF012359, NC004162, FJ445430, FJ445431, FJ445432, FJ445433, FJ445463, FJ445502 and FJ445511). Synthesized biotinylated-peptides were dissolved in dimethyl sulphoxide (DMSO) to obtain a stock concentration of approximately 15 μg/mL. All the peptide samples were screened in triplicates using plasma from either CHIKV-infected patients or healthy donors, as well as in the absence of plasma as described below. Briefly, streptavidin-coated microplates (Pierce) were first blocked with 1% sodium caseinate (Sigma-Aldrich) diluted in 0.1% PBST (0.1% Tween-20 in PBS), before coating with peptides diluted at 1:1,000 in 0.1% PBST and incubated at room temperature for about 1 hour on a rotating platform. Plates were then rinsed with 0.1% PBST before incubation with human plasma samples diluted at 1:200 to 1:2,000 in 0.1% PBST for about 1 hour at room temperature. This was followed by incubation with the respective anti-human IgG and isotype-specific antibodies conjugated to HRP (Molecular Probes) at dilutions from 1:500 to 1:4,000 in 0.1% PBST supplemented with 0.1% sodium caseinate for about 1 hour at room temperature to detect for any antibodies bound to the peptide samples. Binding was detected with TMB substrate solution (Sigma-Aldrich) and color development was stopped with Stop reagent (Sigma-Aldrich). Absorbance was measured at 450 nm using a microplate autoreader (Tecan). Peptides are considered positive if absorbance values are higher than the mean+6 standard deviation (SD) values of negative controls. Structural data was retrieved from PDB (id: 3N44 and 2XFB) and visualized using the software CHIMERA (Pettersen E F, Goddard T D, Huang C C, Couch G S, Greenblatt D M, Meng E C, Ferrin T E, “UCSF Chimera—a visualization system for exploratory research and analysis”, J Comput Chem, 2004, 25, pp. 1605-1612). Solvent excluded molecular surfaces were generated with the help of MSMS package (Sanner M F, Olson A J, Spehner J C, “Reduced surface: an efficient way to compute molecular surfaces”, Biopolymers, 1996, 38, pp. 305-320). Coloring of the E2 domains and orientation of the E1-E2 heterodimer asymmetric unit relative to the viral membrane were based on previously described data (Voss J E, Vaney M C, Duquerroy S, Vonrhein C, Girard-Blanc C, Crublet E, Thompson A, Bricogne G, Rey F A, “Glycoprotein organization of Chikungunya virus particles revealed by X-ray crystallography”, Nature, 2010, 468, pp. 709-712).

Alanine Scanning.

Eighteen peptide sequences were synthesized with substitution of a native amino acid for an alanine (EMC microcollections GmbH). Peptides were dissolved in DMSO to obtain a stock concentration of approximately 15 μg/mL. All the peptide samples were screened in triplicates using plasma from either CHIKV-infected patients or healthy donors. Outputs were expressed as percentage binding capacity relative to the original E2EP3 sequence peptide.

Affinity Depletion of CHIKV Anti-E2EP3 Antibodies.

For affinity depletion of human anti-E2EP3 antibodies, synthetic biotinylated E2EP3 peptide (EMC microcollections GmbH) was added at 450 ng/well to streptavidin-coated plates (Pierce) and incubated at room temperature for about 1 hour in PBS containing 0.1% Tween-20 (0.1% PBST). Human plasma samples were added and incubated for about 25 minutes at room temperature for absorption. The unbound portion was collected after 21 rounds of absorption. ELISA analysis was performed to verify the levels of the antibodies during affinity depletion.

Peptide Blocking Assay.

Synthetic soluble E2EP3 peptide (EMC microcollections GmbH) (100 μg/mL) was mixed with diluted (1:500) heat-inactivated human plasma or serially diluted (from 1:100 to 1:3200) heat-inactivated NHP plasma and incubated for about 1 hour at about 37° C. with gentle agitation (350 rpm). Samples were then mixed with CHIKV at Multiplicity of Infection (MOI) 10 and incubated for about 2 hours at about 37° C. with gentle agitation (350 rpm). Sero-neutralization assay was performed to verify the neutralizing activity

Affinity Depletion of Anti-CHIKV Antibodies.

For affinity depletion of human anti-CHIKV antibodies, purified CHIK virion (1×10⁶ virions/well) were added to Maxisorp plates (Nunc) and incubated at about 4° C. for about 24 hours in PBS. Human plasma samples were added and incubated for about 25 minutes at room temperature for absorption. The unbound portion was collected after 21 rounds of absorption. ELISA analysis was performed to verify the levels of the antibodies during affinity depletion.

Affinity Depletion of Human Isotype IgG3 Antibodies.

For affinity depletion of human isotype IgG3 antibodies, mouse biotinylated monoclonal anti-human IgG3 antibodies (30 μg/mL, Molecular Probes) were added to Immobilizer Streptavidin plates (Nunc) and incubated at room temperature for about 1 hour in PBS containing 0.02% Tween-20 (0.02% PBST). Human plasma samples were added and incubated for about 25 minutes at room temperature for absorption. The unbound portion was collected after 21 rounds of absorption. ELISA analysis was performed to verify the levels of the antibodies during affinity depletion.

Recombinant CHIKV Plasmids.

Codon-optimized C-terminal FLAG-tagged cDNA clones encoding for CHIKV capsid, E2 and E1 were generated (Genscript Corporation) and sub-cloned into pcDNA3.1 expression vector (Invitrogen) to form the pcDNA-C-FLAG, pcDNA-E2-FLAG, and pcDNA-E1-FLAG expression plasmids respectively. Positive clones containing full-length inserts were screened by restriction analysis and confirmed by DNA sequencing.

Rhesus Macaques Studies.

Five-year-old cynomolgus macaques (Macaca fascicularis) were imported from Mauritius. All animals were negative for SIV, Simian T-Lymphotropic Virus, Herpes B virus, filovirus, SRV-1, SRV-2, measles, dengue and CHIKV, and were maintained in a biosafety level 3 facility. Studies were approved by the regional animal care and use committee (“Comite Regional d'Ethique sur l'experimentation animale Ile de France Sud”, Fontenay-aux-Roses, France), reference number: 07-012, in accordance with European directive 86/609/EEC. Animals were infected with 10⁶ PFU (in 1 ml PBS) LR2006-OPY1 CHIKV by I.V. inoculation, as described in Labadie K, Larcher T, Joubert C, Mannioui A, Delache B, Brochard P, Guigand L, Dubreil L, Lebon P, Verrier B, et al., “Chikungunya disease in nonhuman primates involves long-term viral persistence in macrophages”, J Clin Invest, 2010, 120, pp. 894-906. Animals were bled and observed daily for one week than twice a week to assess viral replication, inflammation and clinical signs of infection. No virus was detected in plasma samples at 9 and 13 days post inoculation.

Mouse Studies, Vaccination and Virus Plaque Assay.

Lyophilized KLH-E2EP3 peptide was dissolved in DMSO (Sigma-Aldrich) to a working concentration of 5 mg/mL. Three-weeks old, female, C57BL/6J (sample size, n=7) were vaccinated subcutaneously in the abdominal flank with 100 μg of KLH-E2EP3 peptide prepared in 100 μl emulsion with 50% Complete Freund's Adjuvant (CFA) (Sigma-Aldrich) in PBS. Vaccinated mice were further boosted another two times at day 14 and day 21 with 50 μg of the peptide prepared in Incomplete Freund's Adjuvant (IFA) (Sigma-Aldrich). Control mice (n=7) were vaccinated with PBS/CFA and PBS/IFA on first vaccination and subsequent booster shots respectively. Sera were collected from all mice at day 19 and day 27 post-vaccination for downstream E2EP3 peptide-based ELISA. All protocols were approved by the Institutional Animal Care and Use Committee of the Agency for Science, Technology and Research (A*STAR), IACUC number: 080383. At day 30, C57BL/6J mice from E2EP3-vaccinated and PBS-control groups were inoculated with 10⁶ PFU (in 50 μl PBS) SGP11 CHIKV. Virus was inoculated in the subcutaneous (s.c.) region at the ventral side of the right hind footpad, towards the ankle. Viremia and degree of inflammation were monitored. Viremia analysis was performed for day 2 and day 6. Ten μl of blood was collected from the tail of each mouse in 1 μl of citrate and 89 μl of Hank's buffer (Sigma-Aldrich) and serially diluted up to 10⁻³ times with Hank's buffer. Vero E6 cells were pre-seeded at 2.5×10⁵ cells per well in 24-wells plate and incubated at about 37° C. for about 20 hours. Ninety (90) μl of diluted virus mix was inoculated into each well and incubated for about 1 hour at about 37° C. Virus overlay was removed and the infected monolayers were washed once with 1 ml of sterile PBS. One ml of 1% w/v carboxymethylcellulose (Calbiochem) in DMEM with 5% FBS was then added onto the infected monolayers. Plates were incubated at about 37° C. with 5% CO₂ for about 72 hours and visualized by staining the monolayer with 1 ml of 0.1% w/v crystal violet (Sigma-Aldrich)/10% v/v formaldehyde (Sigma-Aldrich) for about 2 hours at room temperature. Hind footpads of mice were measured daily using a vernier calliper from day 0 to day 14 post-infection. Measurements were done for the height (thickness) and the breadth of the foot and quantified as [height×breadth]. Degree of inflammation was expressed as relative increase in footpad size as compared to pre-infection with the following formula: [(day x−day 0)÷day 0] where x is the footpad measurements for each respective day post-infection.

Data (or Statistical) Analysis.

Data are presented as mean±standard error mean (SEM) or as mean±standard deviation (SD). Differences in responses among groups at various time points and between groups and controls were analyzed using appropriate tests (Mann-Whitney U test, Fisher's exact test, Kruskal-Wallis with Dunn's post-test, One-way ANOVA with Tukey post-test, Two-way ANOVA with Bonferroni's multiple comparisons test). Statistics were performed with GraphPad Prism 5.04.

### Timing and Isotype-Specificity of the Antibody Response

CHIKV-specific antibody responses for 30 infected individuals collated during the CHIKV outbreaks in late 2008 to 2009 were studied. It was also assessed whether an isotype-specific antibody response was correlated with the neutralizing activity in vitro, disease severity and patients' viral load.

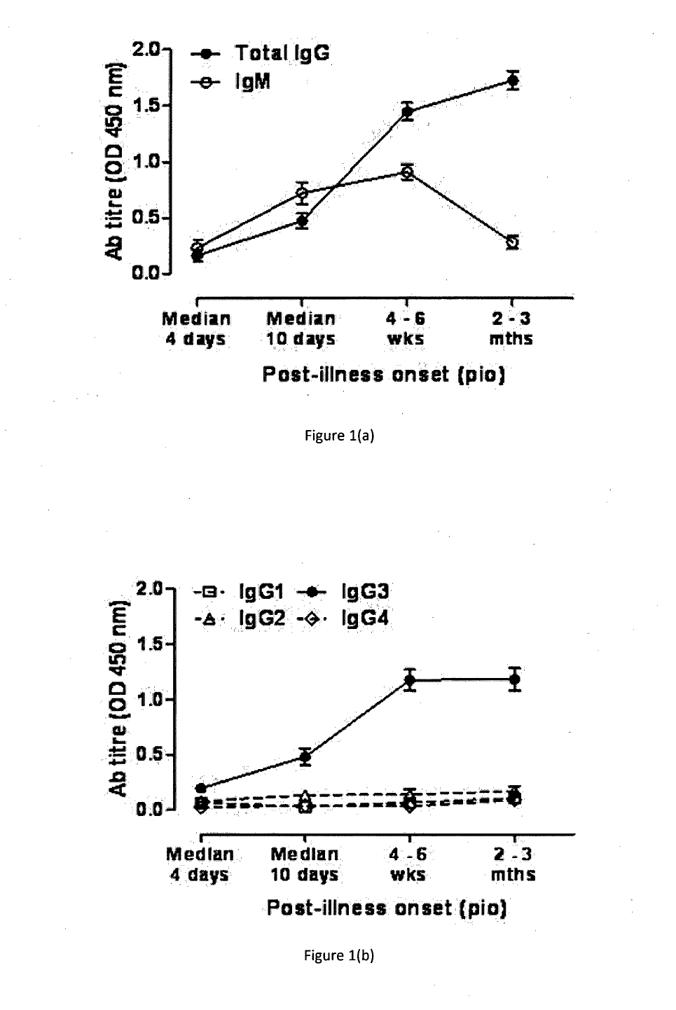

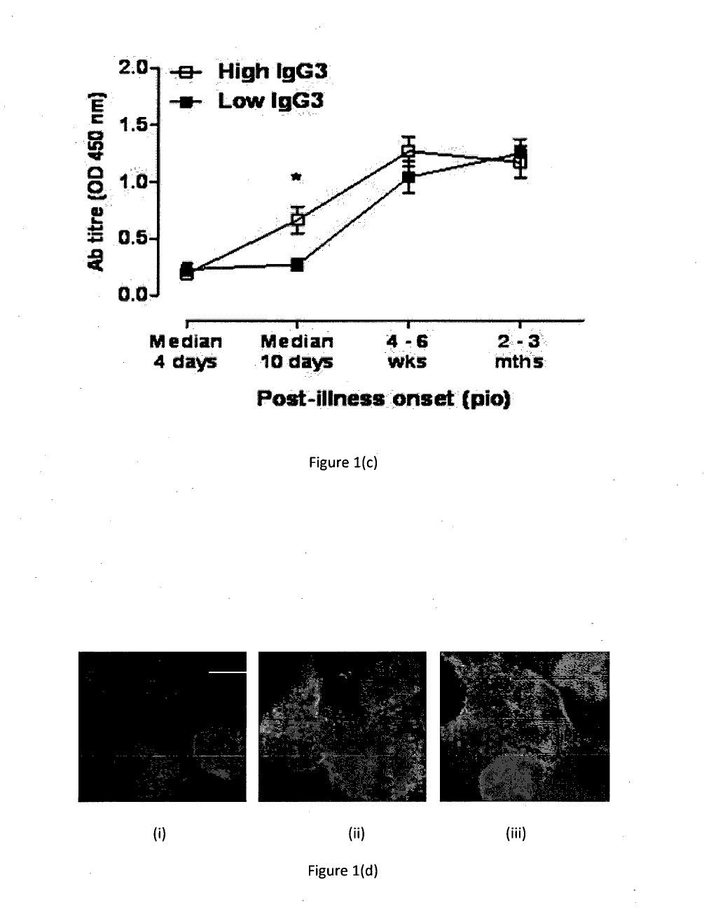

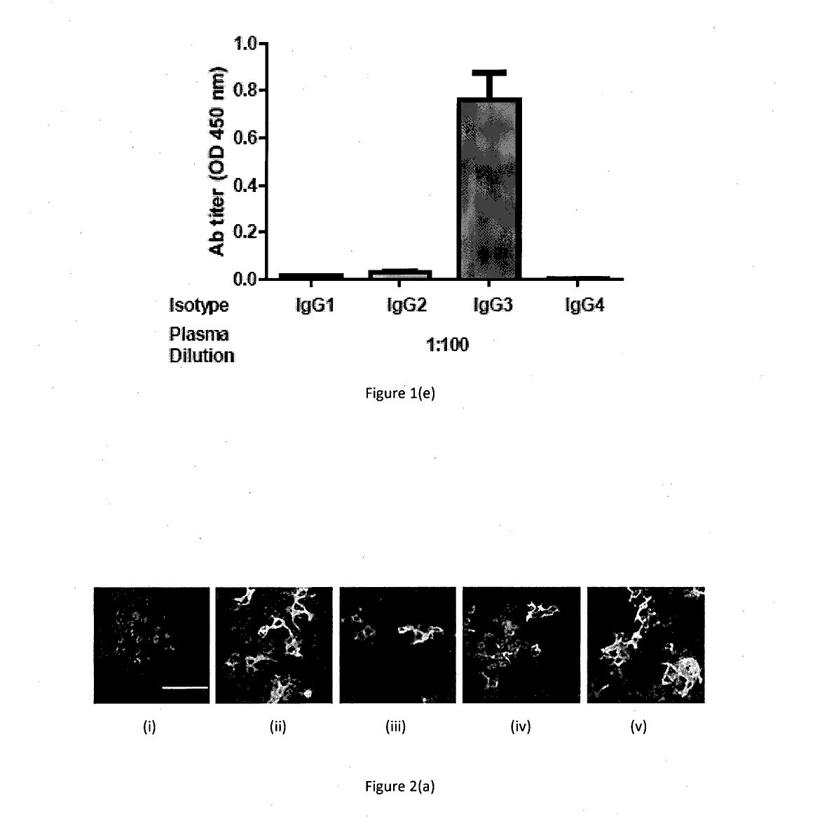

FIG. 1 shows antibody responses and isotyping of CHIKV-infected patients, in accordance to various embodiments.

The antibody kinetics of anti-CHIKV specific IgM and IgG antibodies during the course of illness were studied. It was demonstrated that a transient anti-CHIKV IgM antibody response in the acute phase of illness and a classical switch of Ig antibodies from IgM to IgG was observed at the convalescent phase (FIG. 1(a) depicting the total IgG and IgM).

In FIG. 1(a), virus-specific IgM and IgG antibody titers in plasma samples (n=30), at a dilution of 1:2,000 were determined by ELISA using purified CHIKV virions.

The distribution of CHIKV-specific antibodies among the four subclasses was studied by ELISA. IgG3 antibody was the dominant isotype upon CHIKV infection (FIG. 1(b) depicting the isotype specific IgG). FIG. 1(b) shows virus-specific IgG isotype titers in plasma samples. IgG1 (□), IgG2 (Δ), IgG3 () or IgG4 (♦) were determined as in FIG. 1(a) using specific secondary antibodies. Relatively low levels of IgG 1, IgG2 and IgG4 antibodies persisted throughout the course of illness (FIG. 1(b)).

FIG. 1(c) shows a profile of IgG3 levels at different time post-illness onset in Early IgG3 (n=16) and Late IgG3 responders (n=14) according to the pattern of IgG3 titer at median 10 days. Data are presented as mean±SEM. Data are representative of two independent examples with similar results. Statistical significance was measured using Mann-Whitney U test (** represents P<0.01). Interestingly, two clusters were observed according to the pattern of anti-CHIKV IgG3 antibody response (FIG. 1(c)). Two-way ANOVA analysis showed a significant difference between cluster 1 (Early IgG3 level at 7-10 days pio) and cluster 2 (Late IgG3 level at 7-10 days pio). The important role of anti-viral IgG3 antibody neutralizing potency was observed with other viruses, including measles and HIV. This was the first report demonstrating the induction of a specific human antibody isotype, IgG3, upon CHIKV infection.

FIG. 1(d) shows detection of CHIKV by plasma from CHIKV-infected patients. HEK 293T cells were infected with CHIKV (SGP11) at MOI 10, fixed at about 6 hour post infection and stained with two representative patients' plasma (i.e., (ii) Patient A and (iii) Patient B) at 2-3 months pio, using a dilution of 1:500. Healthy plasma (FIG. 1(d)(i)) was used as a control. CHIKV antigen was detected by anti-human IgG antibody conjugated to Alexa Fluor 488 (green). DAPI was used to stain the nucleus. Scale bar: 10 μm.

FIG. 1(e) shows CHIKV virion-based ELISA being used to determine virus-specific IgG isotype titers in plasma samples (Median 10 days pio, n=30) at a dilution of 1:100. Anti-CHIKV IgG1, IgG2, IgG3 or IgG4 antibodies were determined using specific secondary antibodies. Data are presented as mean±SEM and are representative of two independent examples with similar outcomes.

In order to characterize the immune response against CHIKV, prospective follow-up with 30 patients who were admitted for acute CHIKF during the CHIKF outbreak in Singapore between August and September 2008 were conducted. CHIKV-specific antibody responses were quantified in the acute phase starting 4 days after infection until the late chronic phase 2-3 months post-infection. As expected IgG levels gradually increased during the early convalescent phase at median 10 days post-illness onset (pio) while IgM peaked after 4-6 weeks and declined to background levels, as seen in FIG. 1(a). Plasma from these patients was not only reactive to the CHIKV virion-based ELISA, but also specifically detected CHIKV antigens in CHIKV-infected cells by immunofluorescence staining (FIG. 1(d)).

CHIKV-specific IgG antibodies were found to be almost exclusively of the IgG3 isotype. The levels of virus-specific IgG1, IgG2 and IgG4 titer did not increase during the course of infection (FIG. 1(b)) even when high concentration of plasma was used (FIG. 1(e)). While IgG3 was the dominant isotype in all members of the cohort, a comparison of the individual titers during the early convalescence phase revealed striking differences within the patient group. At median 10 days pio, approximately only half of the group had already a significant increase of IgG3, segregating this study cohort into “early IgG3” and “late IgG3” responders (FIG. 1(c), Table 1).

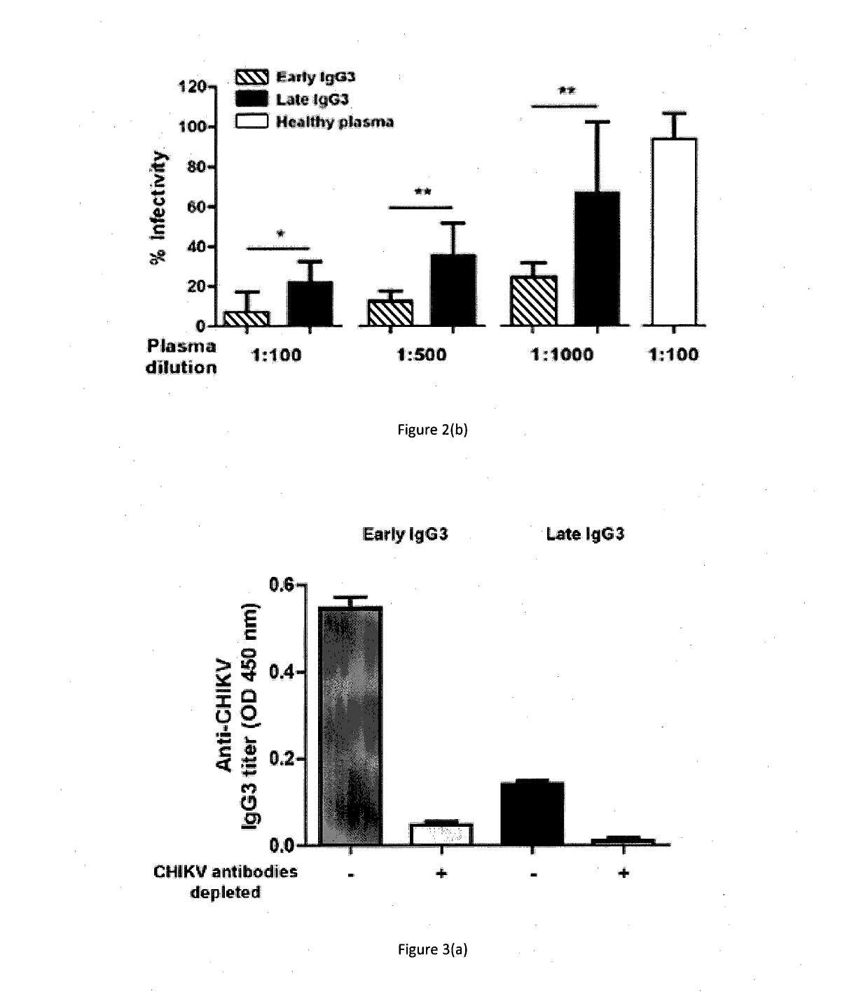

FIG. 2 shows neutralizing activity of CHIKV-infected patient plasma samples in vitro, in accordance to various embodiments.

Patients' plasma samples collected at median 10 days and 2-3 months post-infection were tested for neutralization effects against CHIKV infection in vitro using the high throughput immunofluorescence-based cellomics platform as seen in FIG. 2(a) depicting cases of (i) mock; (ii) no plasma; (iii) low IgG3; (iv) high IgG3; and (v) healthy plasma. Virus samples were pre-incubated with patients' plasma collected at median 10 days pio from Early and Late IgG3 groups before being added to the cells. Non-infected (mock) or virus samples pre-incubated with healthy donor plasma were used as controls. Analysis was performed at about 6 hours post-infection. Images were captured with 60× magnification. Scale bar: 50 μm. Representative microscopic images per treatment condition are illustrated.

The evaluations were interpreted as the percentage of infection between wells infected with immune complexes (virus+patient sample) and wells infected with only virus (data not shown). FIG. 2(b) shows that plasma samples demonstrated neutralizing response in a dose-dependent manner in vitro.

In FIG. 2(b), in vitro neutralizing activity against CHIKV from plasma samples of Early and Late IgG3 responders for Median 10 days pio is provided. Plasma samples (Median 10 days pio) were tested in triplicates at different dilutions. Healthy plasma was used as a control and performed in the same conditions. Dilution at 1:100 is shown. Outputs were presented as mean±SD of percentage control infection. Data are representative of three independent examples. Statistical significance was measured using Mann-Whitney U test (* represents P<0.05; ** represents P<0.01).

Early (high) IgG3 responders showed strong neutralizing response during the early convalescent phase of disease. However, strong neutralizing response was developed only at the later convalescent phase of disease in Late (low) IgG3 responders.

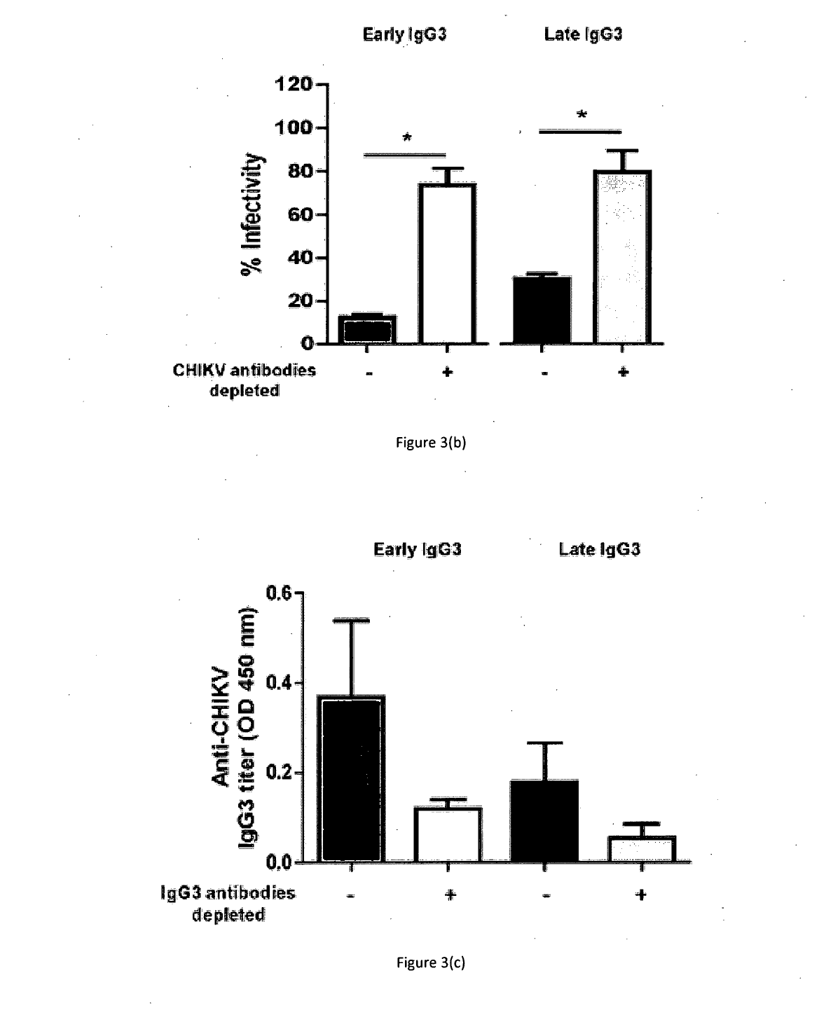

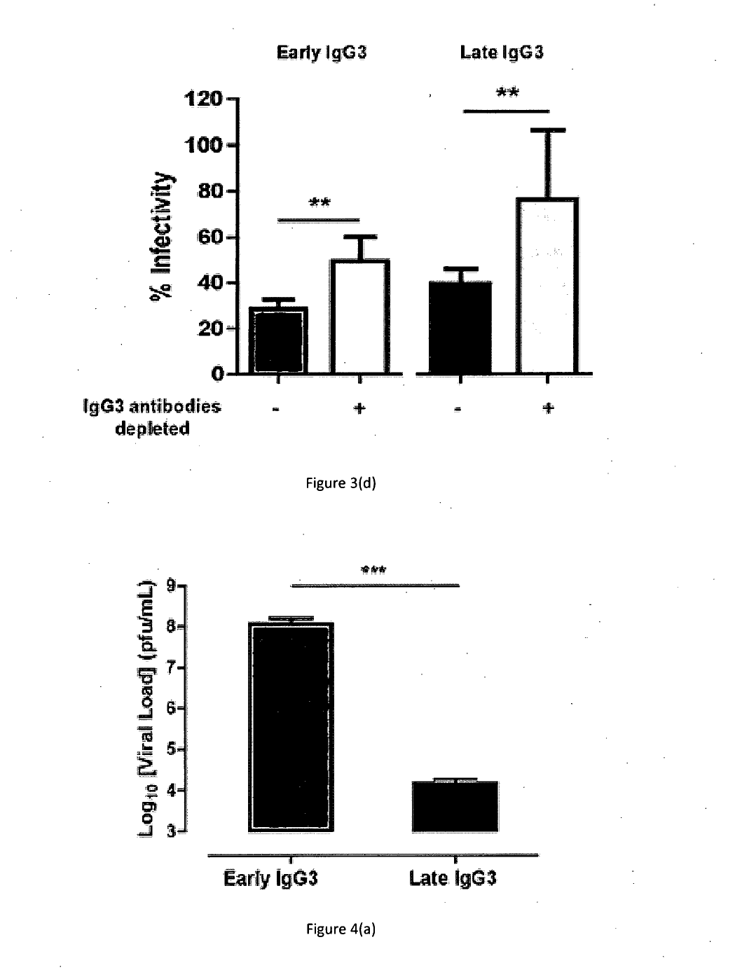

FIG. 3 shows isotype specific anti-CHIKV antibodies have neutralizing activity in vitro, with respective standard deviations, in accordance to various embodiments.

The mechanism of anti-CIHKV antibodies neutralization was demonstrated by depletion example. Patient plasma samples (High IgG3 and Low IgG3) were depleted according to the methods as described herein and efficiency of anti-CHIKV IgG3 anitbodies depletion was found to be higher than 70%, relative to the undepleted samples.

High IgG3 responders showed strong neutralizing response during the early convalescent phase of disease, at the level similar to the Low IgG3 responders. However, depletion strongly reduced the neutralizing activity of plasma from Low IgG3 responders, as compared to the High IgG3 responders. In this example, “mock” samples which represent non-infected controls, and “SPG11”, which represents Chikungunya virus (Singapore strain) were used. In FIG. 3, the indications “−” and “+” correspondingly represent the initial state(s) and depleted state(s) of the respective plasma samples, and the indication “HC” represents healthy control(s).

To further explain, FIG. 3(a) shows plasma samples being added to plates pre-coated with purified CHIK virion for depletion of anti-CHIKV Abs. Depleted samples were subjected to anti-CHIKV IgG3 antibodies detection with virion-based ELISA. FIG. 3(b) shows depleted samples being subjected to in vitro neutralizing activity detection with a sero-neutralization assay. FIG. 3(c) shows IgG3 antibodies from plasma samples (Median 10 days pio) being depleted and measured for anti-CHIKV IgG3 antibodies with virion-based ELISA. FIG. 3(d) shows depleted samples being subjected to in vitro neutralizing detection in a sero-neutralization assay. All samples assayed were performed at 1:500 dilution (n=3). Outputs were presented as in FIG. 3(b). Plasma from healthy donors was used as negative controls. Data were presented as mean±SD. Data were representative of three independent examples. Statistical significance was measured using Mann-Whitney U test (* represents P<0.05; ** represents P<0.01).

To determine if the antibodies have also protective capacity, in vitro infections of HEK 293T cells with CHIKV were carried out in the presence of plasma from patients or healthy donors (FIG. 2). The examples revealed that plasma samples collected at median 10 days pio effectively inhibited CHIKV infection (FIG. 2(a)). Pre-incubation of CHIKV with plasma samples induced a clear and dose-dependent reduction in the detection of CHIKV antigens (FIGS. 2(a) and 2(b)). In line with the observed differences in IgG3 titer, plasma from early IgG3 responders showed a higher neutralizing activity than plasma from the late IgG3 responders (FIG. 2(b)). To confirm the protective role of anti-CHIKV IgG3 antibodies, CHIKV-infected patient plasma samples were depleted of antibodies against the purified CHIK virion (FIG. 3(a)). Removal of anti-CHIKV IgG3 antibodies led to a marked decrease in neutralization for both Early and Late IgG3 responders (FIG. 3(b)). In addition, the partial removal of IgG3 from the plasma of CHIKV patients by plate-bound anti-IgG3 reduced the IgG3 titer by 70-80% (FIG. 3(c)), led to a marked decrease in neutralization for both early and late IgG3 responders (FIG. 3(d)), at least suggesting or even confirming the importance of IgG3 antibodies in virus neutralization

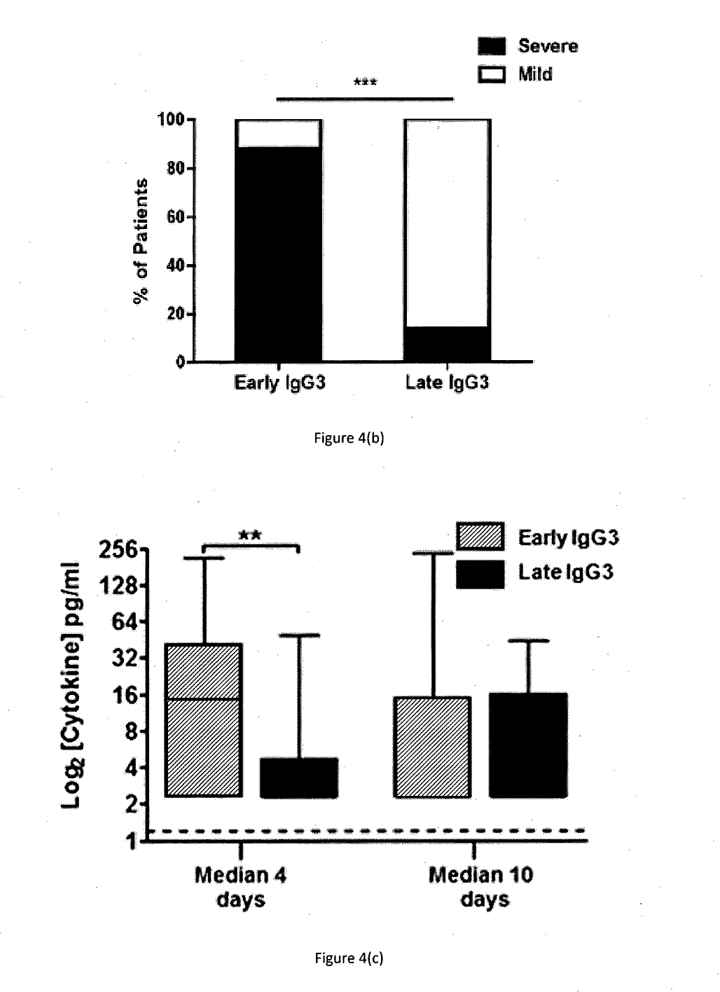

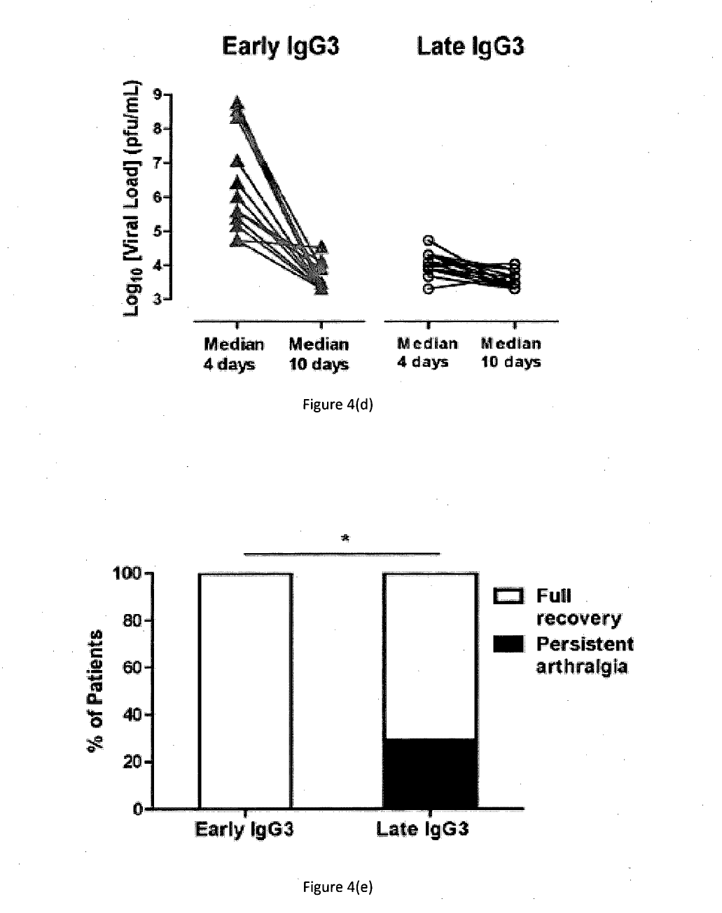

Since CHIKV-IgG3 played a key role in the control of CHIKV infections, viral load and disease progression in early and late IgG3 responders were examined. FIG. 4 shows antibody responses correlate or associate with the disease progression in vivo, in accordance to various embodiments. Data were presented as mean±SEM.

Differences in viral loads, severity and prolonged clinical phenotypes between the two clusters were examined. The high viral loads detected in patient plasma samples during the course of disease, indicated the efficiency of virus replication in vivo. High viremia is correlated to disease severity during the acute phase of illness (FIGS. 4(a) and 4(b) depicting viral load at acute phase of 2 to 4 days pio and percentage of patients with acute severe diseases, respectively).

In FIG. 4(a), viral load in Early IgG3 and Late IgG3 responders during the acute phase of disease (Median 4 days pio) is provided. Data are presented as mean±SD. Statistical significance was measured using Mann-Whitney U test (* represents P<0.05). A much higher viral load in the early IgG3 responders was observed when compared to the late IgG3 responders (FIG. 4(a)). This was particularly evident on median day 4 pio, suggesting that the high IgG3 titers of early IgG3 responders were indeed induced by a high viremia.

In FIG. 4(b), disease severity in Early (High) IgG3 and Late (Low) IgG3 responders during the acute phase of disease. Severity was previously defined. the histogram shows the percentage of patients having mild (n=14) or acute severe clinical phenotypes (n=16). Statistical significance was measured using two-sided Fisher's exact test, between the number of patients with severe phenotype in two responder groups (*** represents P<0.0001).

About 90% of early IgG3 responders were observed to develop severe disease during the acute phase of the infection compared to less than 10% of late IgG3 responders (FIG. 4(b)). In this cohort, disease severity was previously shown to be associated with increased plasma levels of two known endogenous pyrogens IL-113 and IL-6 (Ng L F, Chow A, Sun Y J, et al., “IL-1beta, IL-6, and RANTES as biomarkers of Chikungunya severity”, PLoS One 2009, 4, e4261; and Chow A, Her Z, Ong E K, et al., “Persistent arthralgia induced by chikungunya virus infection is associated with interleukin-6 and granulocyte macrophage colony-stimulating factor”, J Infect Dis 2011, 203, pp. 149-157).

FIG. 4(c) shows IL-6 levels in Early IgG3 and Late IgG3 responders that were determined using a multiplex-bead based assay. Horizontal dotted lines represent median values of healthy controls. Statistical significance was measured using Mann-Whitney U test (** represents P<0.01).

Interestingly, high levels of IgG3 in Early IgG3 responders also correlated with higher IL-6 levels especially during the initial phase of infection (median 4 days pio) (FIG. 4(c)). IL-6 being one of the major B-cell growth factor and an inducer of IgG3, may explain this finding.

In addition, Early IgG3 responders were observed to show limited in vivo virus replication; as compared to Late IgG3 responders (FIG. 4(d)).

Comparison of the viral load on median 4 and 10 days pio indicated that early IgG3 responders exhibited a very efficient clearance of CHIKV. While the average viral load on day 4 differed by more than 3 logs, they reached similar low levels as the late IgG3 responders after completing the acute phase of infection at median 10 days pio (FIG. 4(d)). Thus, the early increase of IgG3 was apparently associated with an efficient clearance of the virus.

Early IgG3 responders showed efficient viral clearance during the acute phase of disease; as compared to Late IgG3 responders (FIG. 4(e)). In FIG. 4(e), persistent arthralgia in Early IgG3 and Late IgG3 responders during the chronic phase of disease (2-3 months pio) is provided. The histogram showed the percentage of patients with full recovery or persistent arthralgia. Statistical significance was measured using two-sided Fisher's exact test, between the patients who have fully recovered and patients who still have persistent arthralgia in the two responder groups (* represents P=0.0365). This may be attributed to a differential IgG3 antiviral response during the acute phase of disease. The correlation between the IgG3 expression and viral clearance was analyzed within the early stage of illness.

Notably, while Early IgG3 responders develop more severe symptoms during the acute phase, they completely recovered from the infection. None of them developed any persistent arthralgia (FIG. 4(e)). This, however, was not the case for Late IgG3 responders. Despite having a low viremia, about 30% of this group developed arthralgia during the later stage of the disease (FIG. 4(e)). This may suggest that a strong early IgG3 response triggered by a high viral load is needed to fully protect against chronic long-term effects of the CHIKV infection.

A therapeutic agent for Chikungunya virus comprising a peptide having the sequence of Capsid and E2 glycoprotein, or a variant thereof having at least 70% amino acid identity therewith, or a fragment thereof having at least 15 amino acid residues, or a derivative thereof, wherein said variant, fragment or derivative has a common antigenic cross-reactivity to said isolated peptide,

Group of 95 possible amino acid sequences (about 18 amino acids in length) generated from the with the amino acid sequences of the Capsid and E2 glycopotein to select from for the Chikungunya-associated peptide.

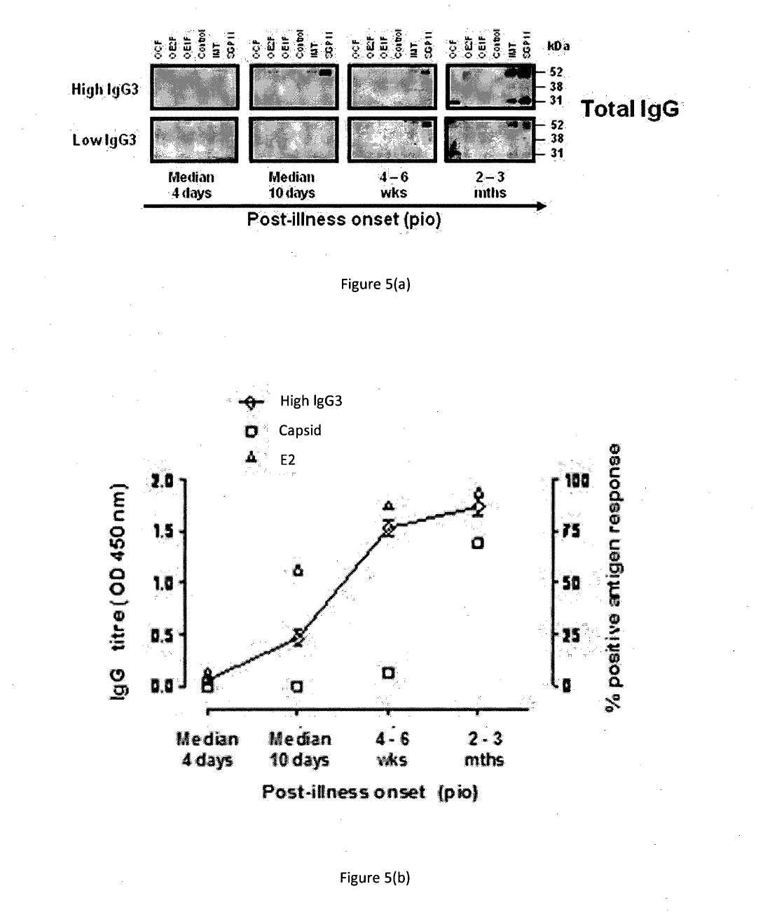

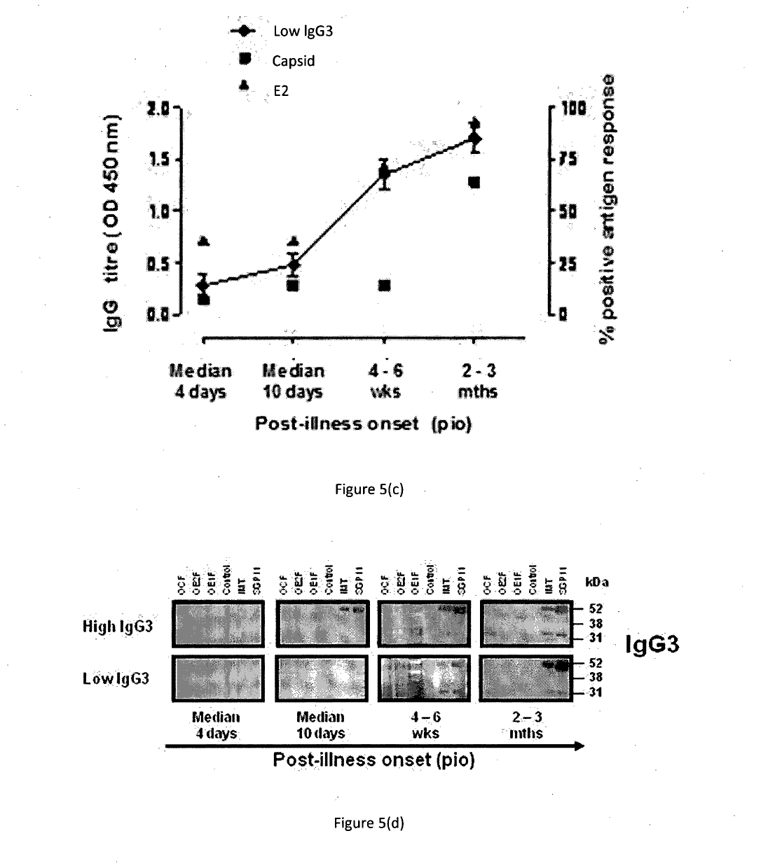

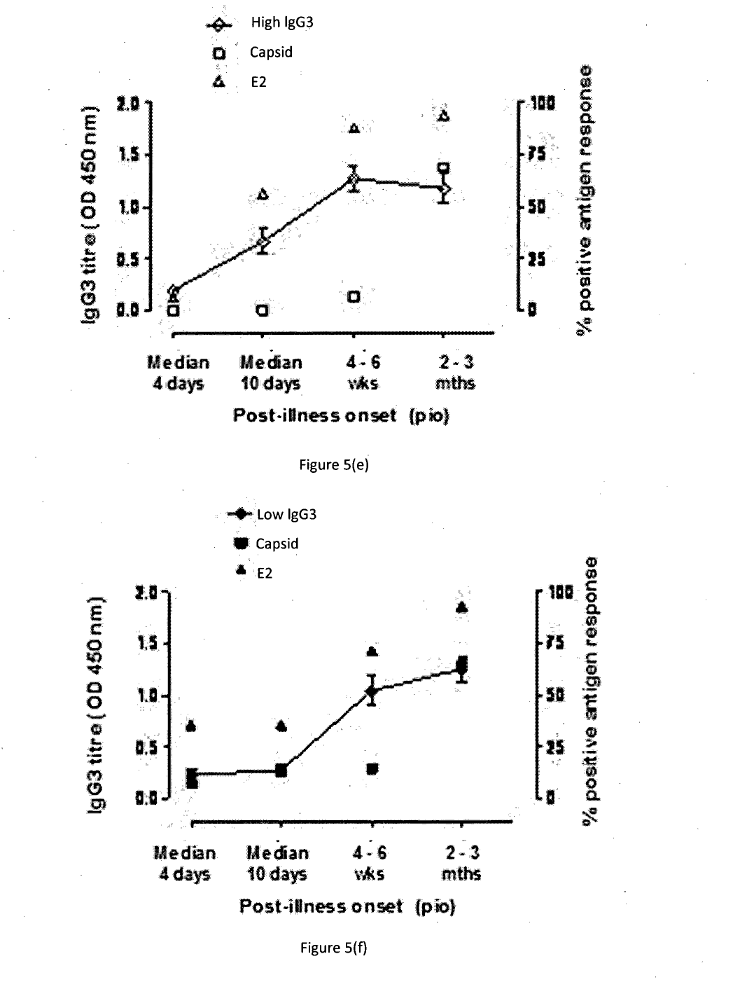

FIG. 5 shows Capsid and E2 glycoproteins contributed to the antigenic responses in vivo, in accordance to various embodiments.

During the acute phase of disease, using computational mapping tools and protocols, it was found that the glycoprotein, E2 was the immunodominant viral protein upon CHIKV infection. Immune response against the capsid was observed only during convalescence whereas anti-E1 antibody response was undetected in any sample (FIG. 5(a)).

The percentage of patients who showed immune responses against E2 glycoproteins correlated positively with the anti-CHIKV IgG response (FIG. 5 depicting (b) high IgG3 and (c) low IgG3). Immune response against capsid proteins was observed only at the later stage of disease.

Similarly, IgG3 was determined to be the major IgG subclass corresponding to viral antigen detection (FIG. 5(d)).

As in the case of FIGS. 5(b) and 5(c), the percentage of patients who showed immune responses against E2 glycoproteins correlated positively with the anti-CHIKV IgG3 response (FIG. 5 depicting (e) high IgG3 and (f) low IgG3). Immune response against capsid proteins was also observed only at the later stage of disease.

The following alphaviruses may be targeted for peptide-based therapies:
O'nyong-nyong virus (strain SG650) (Uniprot ID: sp|O90369.1|POLS_ONNVS)
O'nyong-nyong virus (strain Igbo Ora) (Uniprot ID: sp|O90371.1|POLS_ONNVI)
O'nyong-nyong virus (strain Gulu) (Uniprot ID: sp|P22056.1|POLS_ONNVG)
Semliki forest virus (Uniport ID: sp|P03315.1|POLS_SFV)
Ross river virus (strain T48) (Uniprot ID: sp|P08491.3|POLS_RRVT)
Ross river virus (strain NB5092) (Uniprot ID: sp|P13890.1|POLS_RRVN)
Ross river virus (strain 213970) (Uniprot ID: sp|P17517.1|POLS_RRV2)
Mayaro virus (strain Brazil) (Uniprot ID: sp|Q8QZ72.1|POLS_MAYAB)
Sagiyama virus (Uniprot ID: sp|Q9JGK8.1|POLS_SAGV)

Further, thirty-six other CHIKF patients were recruited from the same hospital and a single sample was taken during admission without further follow up. Serum samples were also obtained from fifteen CHIKF patients (median 14 days pio) seen at the University Malaya Medical Centre in Kuala Lumper in 2008-2009. Clinical features definition are as previously described.

### E2 Glycoprotein is the Dominant Antigen Recognized by CHIKV-Infected Patients:

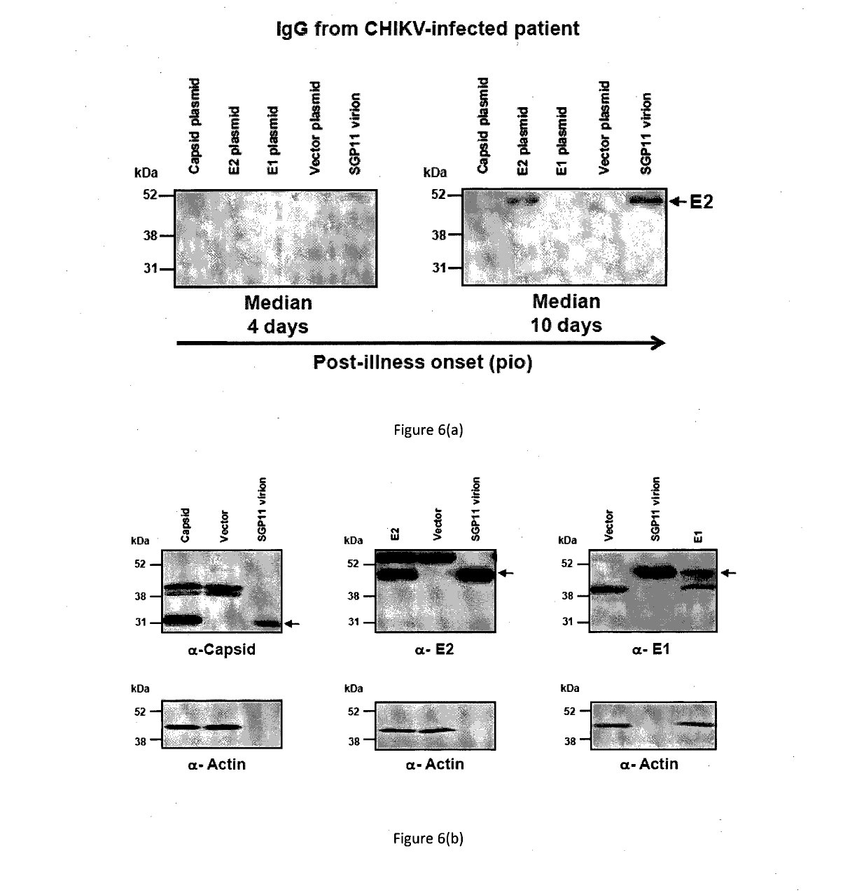

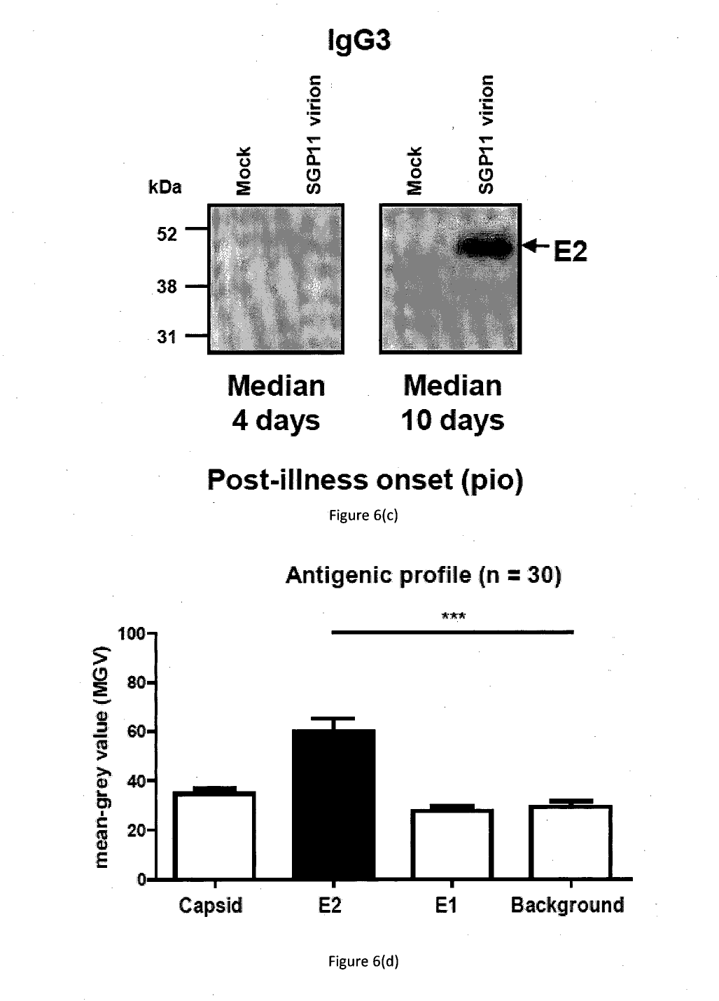

Surface proteins of RNA viruses are targets of neutralizing antibodies. In order to identify which of the surface proteins of CHIKV may be recognized, plasma samples obtained from 30 CHIKV-patients were analyzed. The samples were collected during acute median 4 days post-illness onset (pio) and early convalescent phase (median 10 days pio). Reactivity of each plasma sample was assessed by western blot using purified CHIKV virions (FIG. 6(a)) as well as by lysates of cells transiently expressing recombinant forms of the major CHIKV surface proteins (capsid, E2 and E1 glycoproteins). Identity of the expressed protein was validated with antibodies specific for the respective surface molecule revealing also an accurate molecular weight of about 31 kDa (capsid), 52 kDa (E2) and 51 kDa (E1), as shown in FIG. 6(b).

In FIG. 6(a), total cell lysates were prepared from transiently expressed capsid protein (Capsid plasmid), E2 glycoprotein (E2 plasmid) and E1 glycoprotein (E1 plasmid). Vector transfected (Vector plasmid) cell lysates were used as negative control. Lysates and purified CHIKV virions (SGP11 virion) were subjected to SDS-PAGE gel and probed with a representative CHIKV-infected patient's plasma at a dilution of 1:2,000, followed by secondary human anti-IgG-HRP. Sizes of molecular weight markers are indicated accordingly.

In FIG. 6(b), total cell lysates were prepared from cells transiently transfected with plasmids expressing capsid (Capsid plasmid), E2 (E2 plasmid) and E1 (E1 plasmid). Vector transfected (Vector plasmid) were used as negative controls. Lysates and purified CHIKV virion (SGP11) were subjected to SDS-PAGE and probed with antigen specific polyclonal rabbit antisera (Biogenes) at a dilution of 1:2,000, followed by secondary anti-rabbit IgG HRP antibodies.

IgG may first be measured at the early convalescence time of median 10 days pio, a time point when CHIKV is no longer detectable in the blood. In line with this observation, no specific IgG-bands were evident when using plasma from the acute phase 4 days pio (FIG. 6(a), left panel), whereas a clear IgG-response was detected at median 10 day pio (FIG. 6(a), right panel). Notably, the plasma stained only one specific band corresponding to the E2 glycoprotein. At this time point, no major reactivity was observed for the capsid or the E1 protein, which was consistent for all 30 patients' samples.

FIG. 6(c) shows purified CHIKV virions subjected to SDS-PAGE and probed with CHIKV-infected patients' plasma at 1:1,000, followed by secondary anti-human IgG3 isotype specific antibodies, according to various embodiments.

Quantification of the scanned western blots therefore revealed only for E2 bands intensities that were different from the background (FIG. 6(d)). In FIG. 6(d), band intensities corresponding to CHIKV structural proteins (Capsid, E2 and E1) were analyzed by densitometry for all patient samples (n=30). Outputs were expressed as mean-grey value (MGV)±SD. Data were representative of 2 independent examples with similar results. (*** represents P<0.001 by Kruskal-Wallis test with Dunn's post-test).

Thus, in line with earlier reports on other alphaviruses (Strauss E G, Stec D S, Schmaljohn A L, Strauss J H, “Identification of antigenically important domains in the glycoproteins of Sindbis virus by analysis of antibody escape variants”, J Virol, 1991, 65, pp. 4654-4664; Kerr P J, Fitzgerald S, Tregear G W, Dalgarno L, Weir R C, “Characterization of a major neutralization domain of Ross river virus using anti-viral and anti-peptide antibodies”, Virology, 1992, 187, pp. 338-342; Griffin D, “Roles and reactivities of antibodies to alphaviruses”, Seminars in Virology, 1995, 6, pp. 249-255), E2 glycoprotein is the main target in naturally-acquired immunity in infected patients who just cleared their viremia.

### Identification of Other Epitopes in the E2 Glycoprotein:

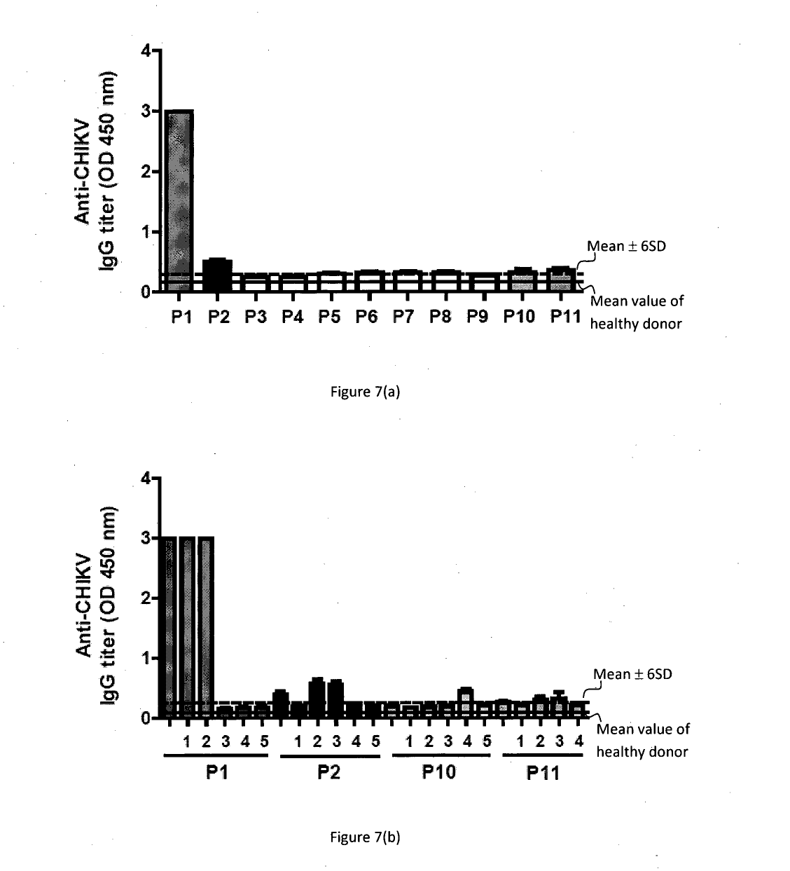

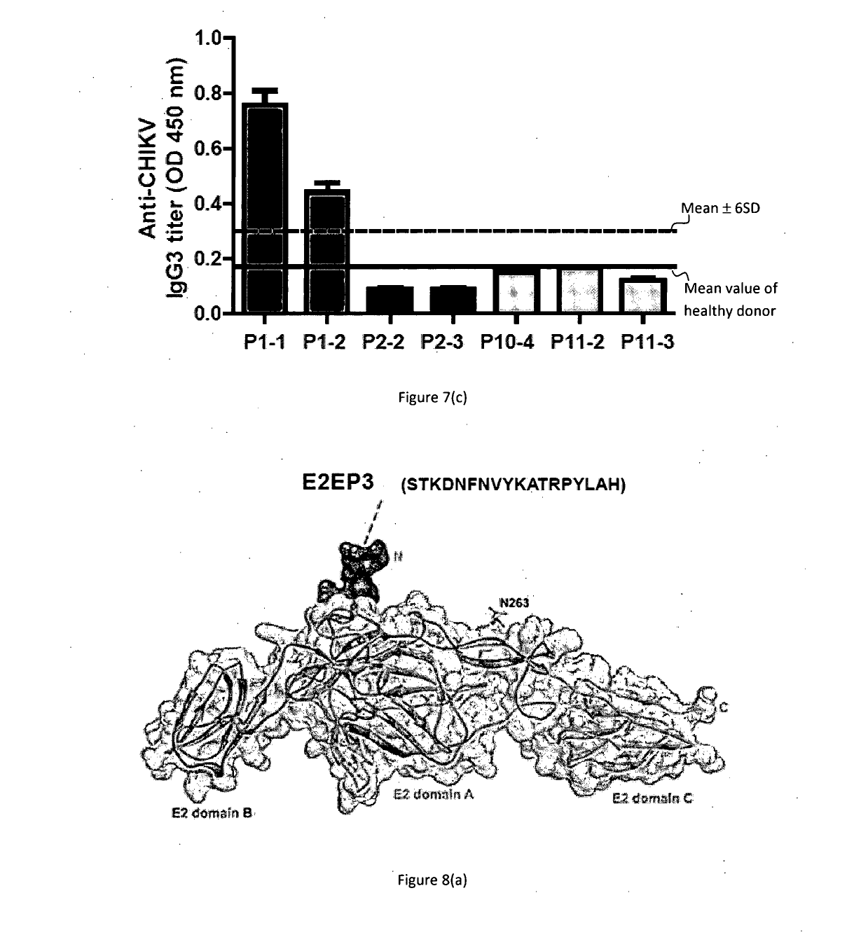

Overlapping peptides corresponding to the CHIKV E2 glycoprotein were screened for antibody binding using patients' plasma. For overlapping peptides, N-terminus region of the E2 glycoprotein starts from pool 1 to pool 11 consecutively to the C-terminus of the protein (FIG. 7). In FIG. 7(a), CHIKV-infected patient plasma pools (Median 10 days pio) were subjected to peptide-based ELISA at a dilution of 1:2,000, followed by secondary human anti-IgG-HRP using pooled peptides (pool 1-pool 11). As described in FIG. 7(b), the same set of patient plasma pools were subjected to peptide-based ELISA at a dilution of 1:2,000, followed by secondary human anti-IgG-HRP using both selected peptide pools (pool 1, pool 2, pool 10 and pool 11) and individual peptides.

With five peptides in each pool. Based on the outputs shown in FIG. 7, seven positive peptide pools were detected. Cutoff values were set at 6 SD (for higher stringency) above the mean of the healthy donors.

Individual peptides from the ‘positive peptide pools’ were re-screened again under the same patients' plasma conditions to determine the specific peptides recognised by the patients' plasma (FIG. 7(c)). Similar cutoff values were used to determine peptide specificity. FIG. 7(c) shows selected individual peptides being re-screened with patients' plasma pools at a dilution of 1:200, followed by secondary human anti-IgG3-HRP. Black solid line represented the mean value of the healthy donors and dotted line represented the value of mean±6 SD. Values above mean±6 SD were considered positive. Ouputs represented an average of 2 independent examples.

In order to identify linear epitopes within the E2 glycoprotein, a peptide library consisting of overlapping peptides was scanned with the pooled patients' plasma (FIG. 7(a)). The library covered the entire E2 glycoprotein and consisted of 18-mer peptides, each with an overlap of 10 amino acids. Analysis of pools combining 5 consecutive peptides revealed that the IgG-response was most pronounced against the N′-terminal part of the E2 glycoprotein (pool 1). Only some minor reactivity was detected to the other regions of the protein (pool 2, pool 10 and pool 11) (FIG. 7(a)). Plasma samples were next assayed with the complete set of single peptides from each of the 4 active pools (FIG. 7(b)). It was found that the antibodies strongly recognized the first 2 peptides of pool 1. In a previous study, it was established that the early IgG response against CHIKV was almost exclusively driven by antibodies of the IgG3 isotype. A very similar picture therefore emerged when anti-IgG3 instead of anti-IgG was used for detection (FIG. 7(c)). Although the sensitivity of the IgG3 assay was generally weaker, the two peptides of pool 1 were clearly detectable, showing a slightly stronger titer for first peptide of P1-1.

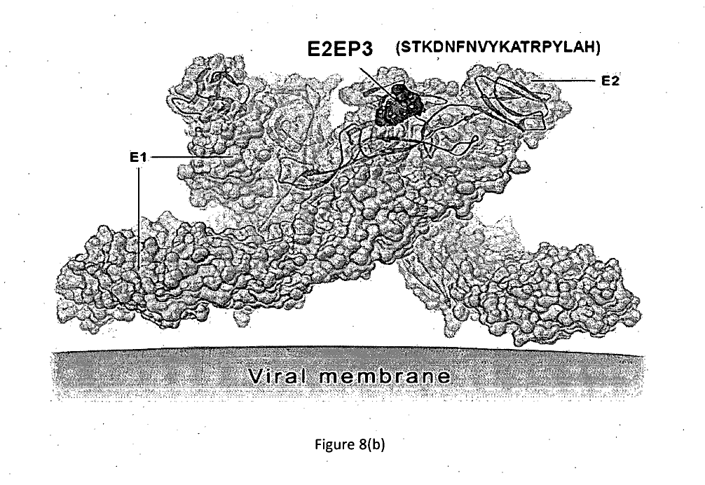

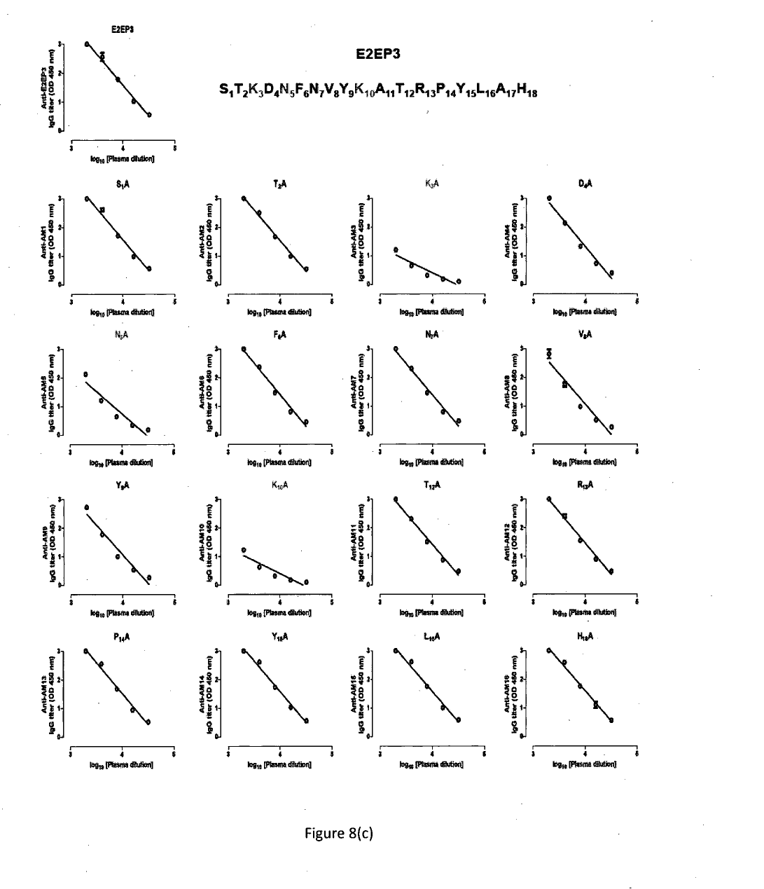

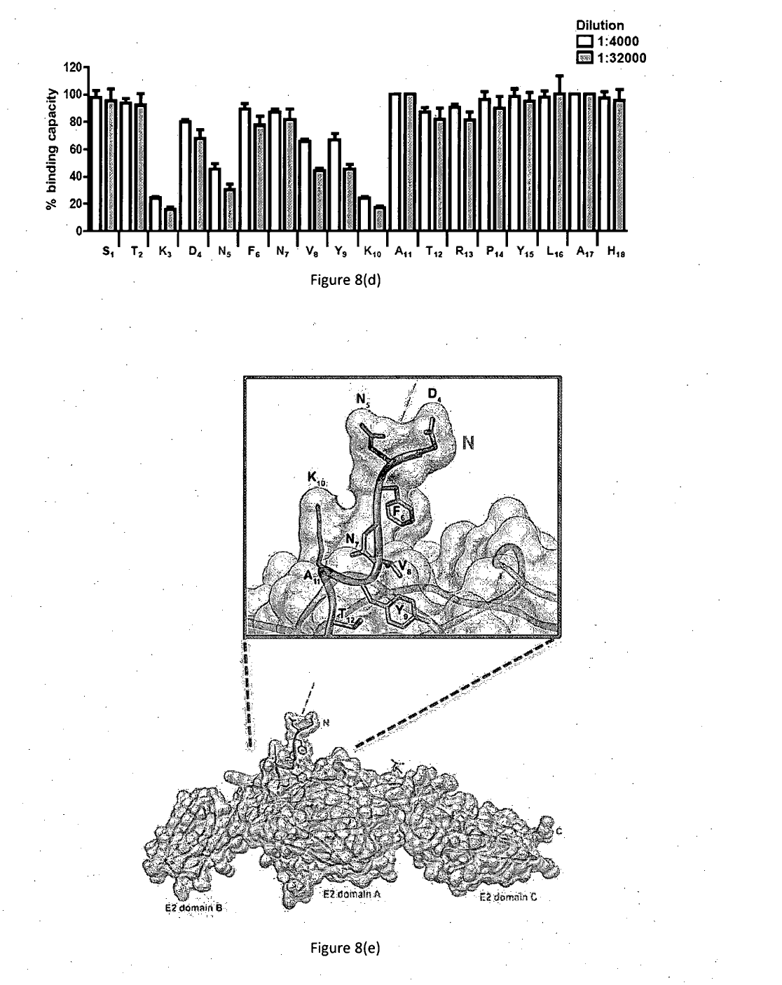

FIG. 8(a) shows a schematic diagram of the localization of the E2 glycoprotein specific epitope (denoted as E2EP3) in the E2 glycoprotein alone based on structural data retrieved from PDB records: 3N44. Tertiary structure of E2 glycoprotein is arranged into three structural domains (E2 domain A-amino terminal; E2 domain B-centre; E2 domain C-carboxyl terminal).

FIG. 8(b) shows a schematic diagram of the localization of E2EP3 in the protein complex situated at the surface of the virus based on structural data retrieved from PDB records: 2×FB. Spatial arrangement of E1 glycoprotein and E2 glycoprotein on the viral membrane surface are indicated accordingly.

The strong response against the first two peptides suggested that the epitope (termed here “E2EP3”) may be present within the overlapping part of peptides (e.g., P1-1 and P1-2 in FIG. 7(c)). The sequence alignment revealed that the overlap (STKDNFNVYKATRPYLAH) was located proximal to the furin cleavage site. The site was required for the proteolytic generation of E2 and E3 glycoproteins from the common precursor protein and the “furin loop” was conserved in alphaviruses. The availability of the recent crystal structure of the CHIKV E1-E2 glycoprotein further allowed the precise localization of E2EP3 epitope. In the mature E2 glycoprotein (FIG. 8(a)), the amino acids of E2EP3 form the N-terminal part of the molecule. This region is prominently exposed on the surface of the virus, forming a stalk that points away from the virus envelope (FIGS. 8(a) and 8(b)).

FIGS. 8(c) to 8(e) show an alanine-scan analysis of E2EP3 by anti-CHIKV antibodies.

Plasma pools (Median 10 days pio) were tested in triplicates at dilutions from 1:2,000 to 1:32,000, as indicated in FIG. 8(c). FIG. 8(c) shows alanine-scan analyses of E2EP3 by anti-CHIKV antibodies. Alanine substitutions were constructed at each position of E2EP3 except the existing alanines. CHIKV-infected patients' plasma pools were used to validate binding capacity. Plasma pools at median 10 days pio were tested in a set of serial dilutions from 1:2000 to 1:32000 and assayed in triplicates. Outputs were expressed as percentage binding capacity relative to the original E2EP3 sequence (% binding capacity) ±SD. Evaluations were performed in triplicates.

In FIG. 8(d), alanine substitutions were constructed at each position of E2EP3 except the existing alanine residues. CHIKV-infected patients' plasma pools were used to validate the binding capacity.

FIG. 8(e) shows a schematic diagram of the localization of the asparagine (N5) and lysine (K10) residues within the E2EP3 epitope region in the E2 glycoprotein based on structural data retrieved from PDB records: 3N44. The structure for K3 was not resolved and therefore could not be localized. Tertiary structure of E2 glycoprotein was arranged into three structural domains (E2 domain A-amino terminal; E2 domain B-centre; E2 domain C-carboxyl terminal). Enlarged image shows the spatial position of the different amino acid residues within E2EP3 with N5 and K10 highlighted in red.

Using a library of peptides containing a series of alanine-substituted amino acids (Cunningham B C, Wells J A, “High-resolution epitope mapping of hGH-receptor interactions by alanine-scanning mutagenesis”, Science, 1989, 244, pp. 1081-1085), both the core-binding region as well as the key amino acids recognized by anti-E2EP3 antibodies of patients' plasma may be identified. The alanine-scan (FIG. 8(d)) showed good correlation with the crystal structure (FIG. 8(e)). Based on this data, the core-binding region of E2EP3 comprises aa3-10 (STKDNFNVYK), which represents the exposed part of the sequence (aa1-3 were not resolved in the crystal structure).

A particularly strong abrogation of binding was observed after replacing residues K₃, N₅ and K₁₀. Their amino acid side chains are either polar (N₅) or positively charged (K₃, K₁₀), and were exposed to solvent in the crystal structure. The substitution of these amino acids reduced antibody binding to below 40% compared to the original E2EP3 peptide (FIG. 8(d)).

### The Neutralizing Effect of Patients' Plasma is Directed Predominantly Against E2EP3:

The neutralizing capacity of CHIKV-specific antibodies in the plasma was tested in vitro. For this, CHIKV were pre-incubated with the pools of patients' plasma before infecting HEK 293T cells. Immunofluorescence staining followed by single-cell quantification using the Cellomics high content screen was used to assess infectivity by determining the number of CHIKV positive cells. Pooled plasma from infected patients effectively neutralized CHIKV infection.

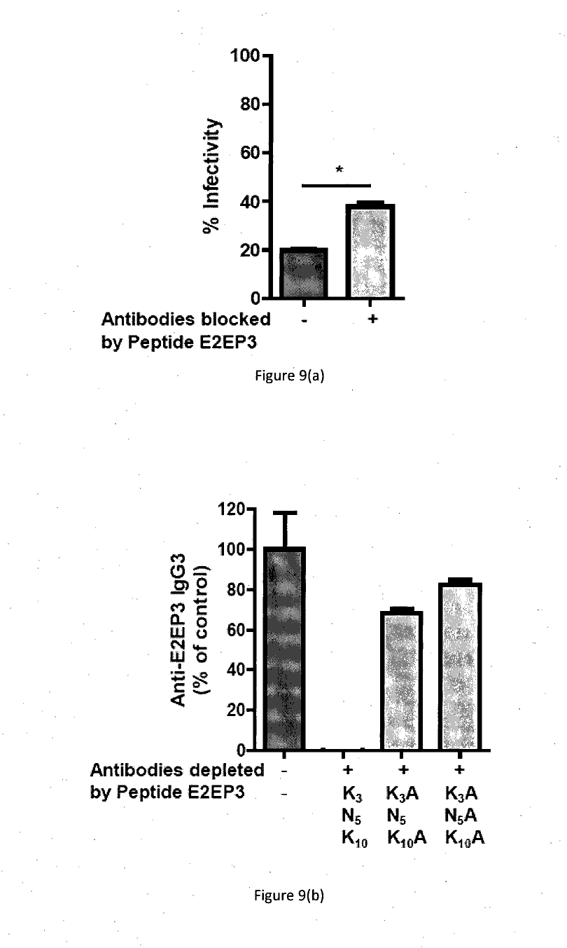

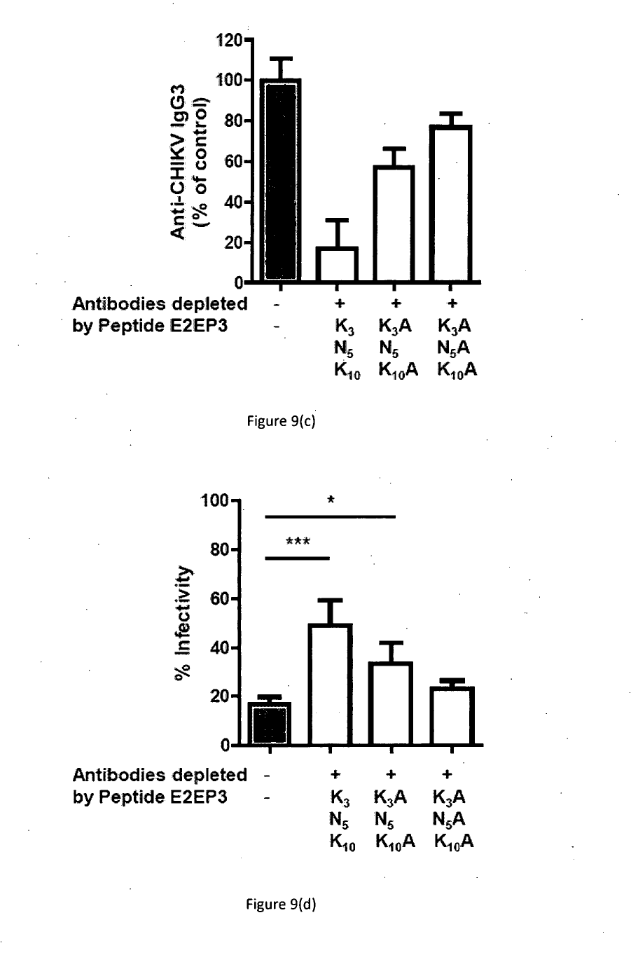

In FIG. 9(a), anti-E2EP3 antibodies in patients' plasma pools were specifically blocked by soluble E2EP3 peptide and followed by in vitro neutralization assay. Outputs were expressed as percentage control infection. Data were presented as mean±SD. Neutralization assays were performed at 1:500 dilution (n=3). (* represents P<0.05, Mann-Whitney U test).

The infection rate decreased to approximately 20% of total cells (FIG. 9(a)). The addition of soluble E2EP3 peptide to the plasma however partially abrogated the neutralization. Blocking with E2EP3 peptide increased CHIKV infection from 20% to almost 40%, at least suggesting or even verifying that antibodies to E2E3P were indeed strongly neutralizing.

This observation was further confirmed in examples where E2EP3-specific IgG3 antibodies were selectively depleted. Exposure of the patients' plasma to surface-bound E2EP3 peptide completely removed all E2EP3-specific IgG3, while a partial depletion was achieved with peptides where the key amino acids K₃, N₅ and K₁₀ were alanine-substituted (E2EP3-specific IgG3 was depleted by 30% for peptide K₃A/K₁₀A, and by 15% for peptide K₃A/N₅A/K₁₀A) (FIG. 9(b)).

In FIG. 9(b), alanine substituted peptides did not deplete E2EP3-specific antibodies in pooled patients' plasma. Plasma samples (Median 10 days pio) were incubated with E2EP3 (K₃, N₅, K₁₀), E2EP3 with double alanine substitution at lysine residues (K₃A, N₅, K₁₀A) or triple alanine substitution at lysine and asparagine (K₃A, N₅A, K₁₀A) peptides. E2EP3 specific peptide-based ELISA was performed to measure the depletion efficiency. Outputs were expressed as percentage control IgG3 titer from non-depleted samples. Data were presented as mean±SD. Examples were performed in triplicates.

The impact of the complete or partial depletion of E2EP3-specific IgG3 antibodies was then tested by comparing the titers of the plasma pools on whole virus (FIG. 9(c)).

In FIG. 9(c), depleted samples as described in FIG. 9(b) were subjected to anti-CHIKV IgG3 antibodies detection. Virion-based ELISA was performed as described to measure the depletion efficiency. Outputs were expressed as percentage control IgG3 titer from non-depleted samples. Data were presented as mean±SD. Examples were performed in triplicates.

The removal of E2E3P-specific antibodies reduced the total anti-CHIKV IgG3 titer by almost 80%. The partial removal by peptide K₃A/K₁₀A decreased the titer by 40%, while peptide K₃A/N₅A/K₁₀A decreased by 20% (FIG. 9(c)). The drastic reduction in the titer indicates that anti-E2EP3 antibodies make up a substantial fraction of the total CHIKV specific IgG3.

The removal of E2EP3-specific IgG3 also directly translated into a reduced neutralization capacity of the plasma pools (FIG. 9(d)).

In FIG. 9(d), in vitro neutralizing activity of anti-E2EP3 antibodies against CHIKV-infected patients' plasma samples was observed. E2EP3 specific antibodies from pooled plasma samples (Median 10 days pio) were depleted by E2EP3 (K₃, N₅, K₁₀), E2EP3 with double alanine substitution (K₃A, N₅, K₁₀A) and triple alanine substitution (K₃A, N₅A, K₁₀A). Neutralization assays were performed at 1:500 dilution (n=3). Non-depleted plasma and healthy plasma were used as controls. Outputs were expressed as percentage control infection. Data were presented as mean±SD. (* represents P<0.05; *** represents P<0.001 by one-way. ANOVA with Tukey post-test).

Depletion of plasma with E2EP3 partly restored virus infectivity from about 20% to more than 50%. As expected, only a gradual decrease of the neutralizing efficacy was observed for the alanine-substituted E2EP3 peptides K₃A/K₁₀A and K₃A/N₅A/K₁₀A (FIG. 9(d)). Thus, during early convalescence, E2EP3 specific IgG3 antibodies largely mediate the neutralizing effect in patients' plasma.

### E2EP3 Specific IgG3 is a Common Marker of Early CHIKV-Infection:

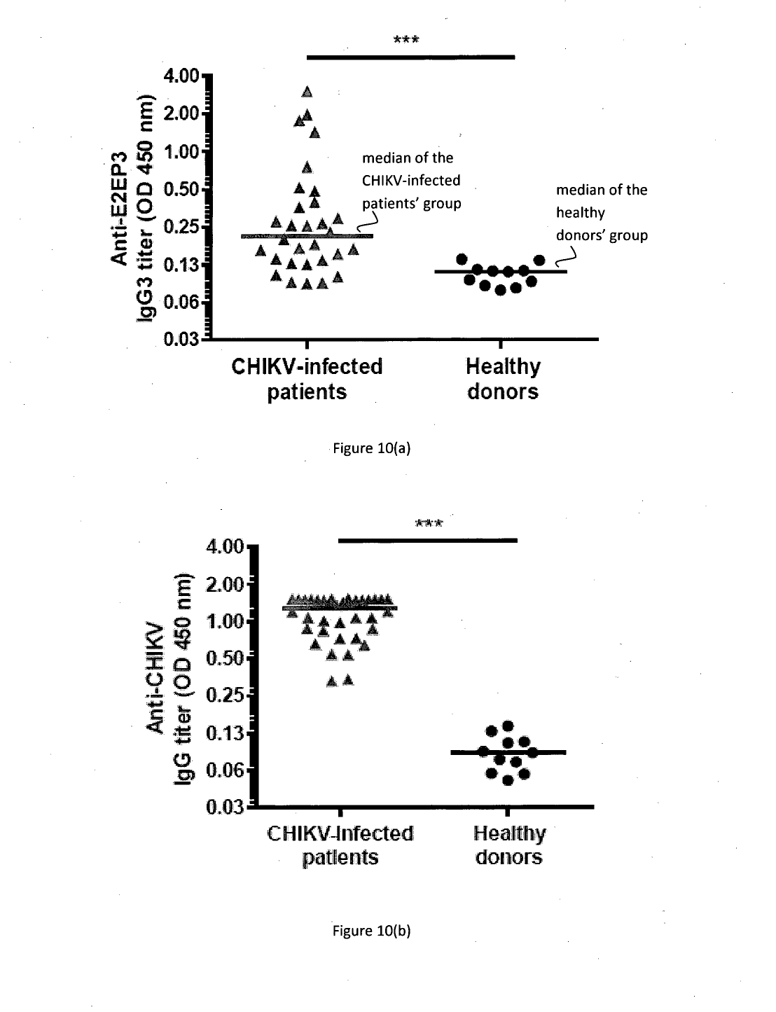

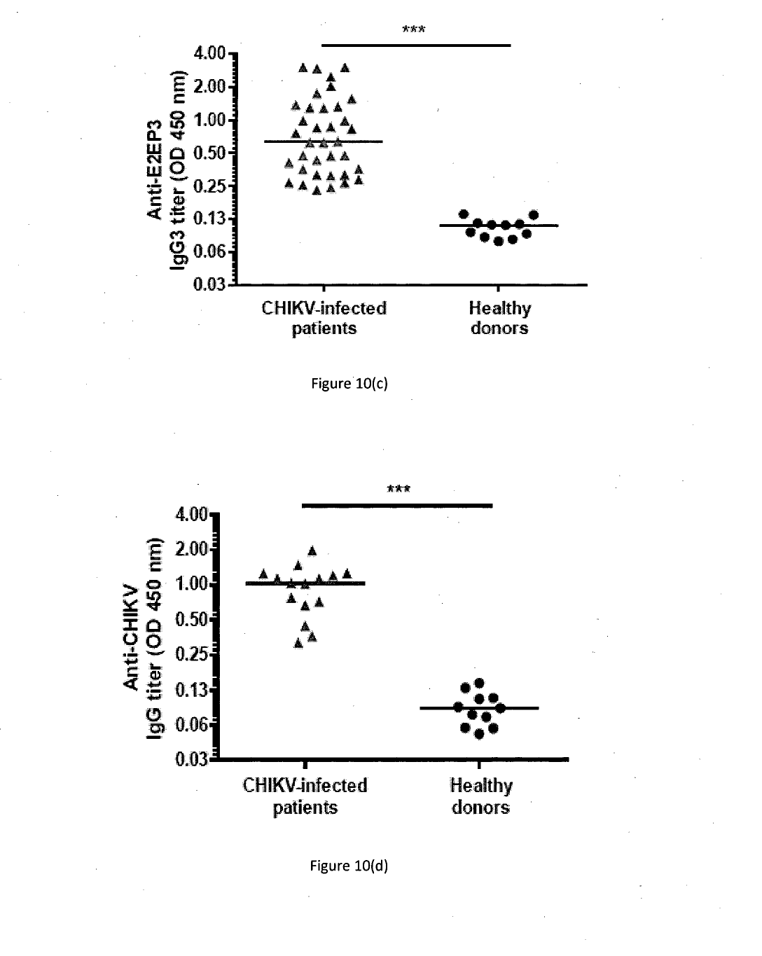

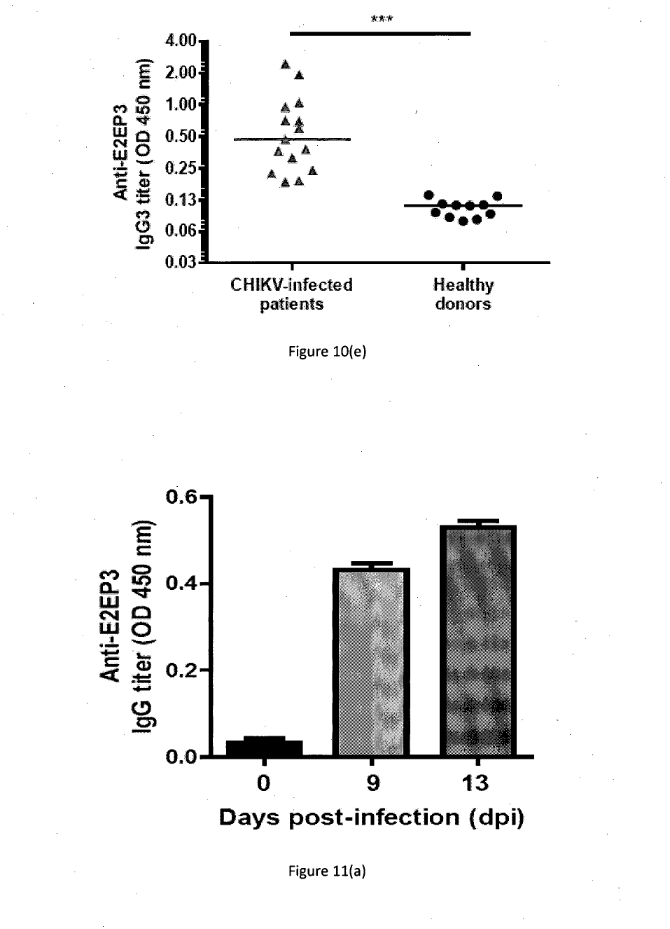

At median 10 days pio, almost all of the patients from this cohort were sero-positive for E2EP3 IgG3 antibodies (FIG. 10(a)).

FIG. 10(a) shows validation of E2EP3 specific IgG3 antibodies in 30 CHIKV-infected patients. Individual plasma samples at median 10 days pio were subjected to E2EP3 specific peptide-based ELISA at a dilution of 1:200, followed by secondary human anti-IgG3 isotype HRP. Healthy donors' plasma (n=11) were used as controls. Samples assayed were performed at triplicate. *** represents P<0.001 by Mann-Whitney U test. The y axis is plotted in log 2 scale. Similar denotes were applicable to FIGS. 10(b) to 10(e).

To further validate the specificity and versatility of E2EP3 as a suitable early detection target, plasma samples were screened from another 36 CHIKV-infected patients collected from a separate cohort together with plasma obtained from 11 healthy donors (FIG. 10). Plasma were again collected during the early convalescent phase (median 10 days pio) and tested for anti-E2EP3 IgG3 antibodies by ELISA (FIG. 10(c)). Whole virus was used as a reference FIG. 10(b)).

In FIG. 10(b), CHIK virion-based ELISA was used to assess anti-CHIKV IgG titer in CHIKV-infected patients from another Singaporean cohort collected at median 10 days pio (n=36). Healthy donors' plasma (n=11) were used as controls. Individual samples were subjected to virion-based ELISA at a dilution of 1:2,000, followed by secondary human anti-IgG-HRP. *** represents P<0.001 by Mann-Whitney U test. Examples were performed in triplicates.

In FIG. 10(c), CHIKV-infected patients' and healthy donors' plasma were screened for IgG3 specific antibodies recognizing E2EP3 in the peptide-based ELISA. Individual samples were subjected to E2EP3 specific peptide-based ELISA at a dilution of 1:200, followed by secondary human anti-IgG3 isotype HRP. *** represents P<0.001 by Mann-Whitney U test. Examples were performed in triplicates.

As in the previous cohort, specific E2EP3-binding was detected in virtually all CHIKV-infected patients with a clear segregation from the sero-negative healthy control donors (FIGS. 10(b) and 10(c)). Similar results were also obtained in a cohort from Malaysia where early convalescence samples of median 14 days pio were collected at outbreaks a few months later (Sam I C, Chan Y F, Chan S Y, Loong S K, Chin H K, Hooi P S, Ganeswrie R, AbuBakar S, “Chikungunya virus of Asian and Central/East African genotypes in Malaysia”, J Clin Virol, 2009, 46, pp. 180-183). Likewise, all of the patients screened were sero-positive for E2EP3, while no reactivity against the epitope was detected in healthy donors (FIGS. 10(d) and 10(e)).

In FIG. 10(d), CHIK virion-based ELISA were used to assess anti-CHIKV IgG titer in 15 CHIKV-infected patients from another cohort collected in Malaysia at median 14 days pio. Healthy donors' plasma (n=11) were used as controls. Individual samples were subjected to virion-based ELISA at a dilution of 1:2,000, followed by secondary human anti-IgG-HRP. *** represents P<0.001 by Mann-Whitney U test. Examples were performed in triplicates.

In FIG. 10(e), CHIKV-infected patients' and healthy donors' plasma were screened for IgG3 specific antibodies recognizing E2EP3 in a peptide-based ELISA. Individual samples were subjected to E2EP3 specific peptide-based ELISA at a dilution of 1:200, followed by secondary human anti-IgG3 isotype HRP. *** represents P<0.001 by Mann-Whitney U test. Examples were performed in triplicates. The same set of healthy donors' plasma comprising of donors from Singapore and Malaysia were used as controls throughout the study. The y axis is plotted in log 2 scale.

Thus, E2EP3 specific IgG3 antibodies appear to be a common early marker for CHIKV-infections at the population level.

### E2EP3 in Pre-Clinical Models—Marker and Vaccine:

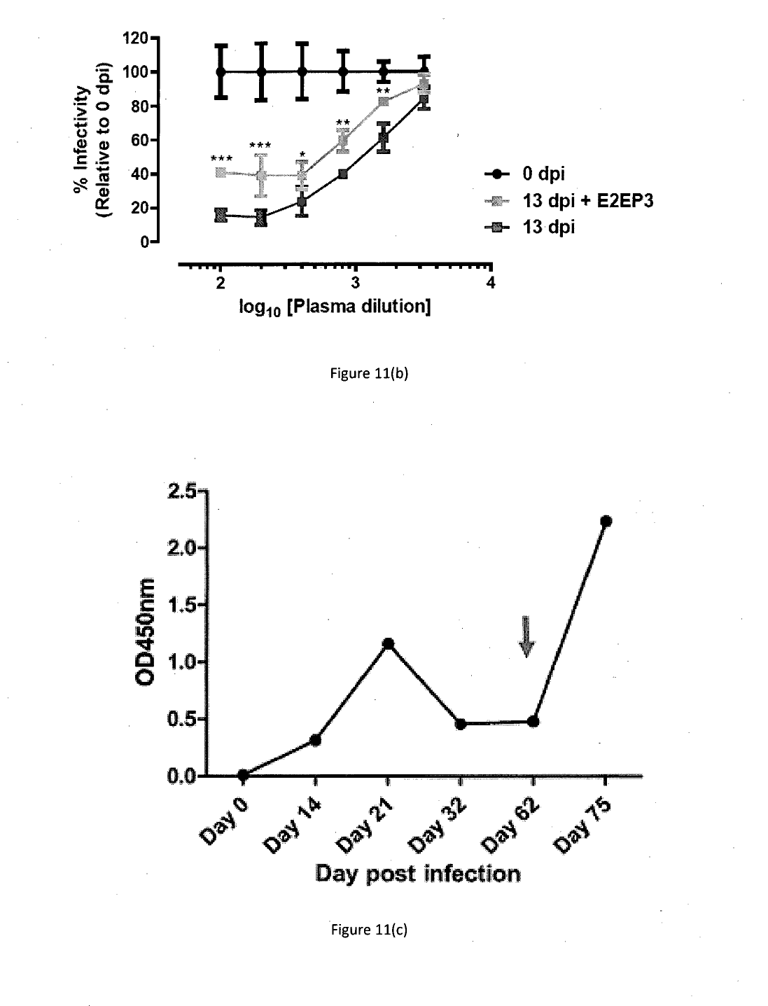

Non-human primates (NHP) are the most relevant and commonly used pre-clinical models for viruses (Liu X, Luo M, Trygg C, Yan Z, Lei-Butters D C, Smith C I, Fischer A C, Munson K, Guggino W B, Bunnell B A, et al., “Biological Differences in rAAV Transduction of Airway Epithelia in Humans and in Old World Non-human Primates”, Mol Ther, 2007, 15, pp. 2114-2123; Morgan C, Marthas M, Miller C, Duerr A, Cheng-Mayer C, Desrosiers R, Flores J, Haigwood N, Hu S L, Johnson R P, et al., “The use of nonhuman primate models in HIV vaccine development”, PLoS Med, 2008, 5, e173; Higgs S, Ziegler S A, “A nonhuman primate model of chikungunya disease”, J Clin Invest, 2010, 120, pp. 657-660; Labadie K, Larcher T, Joubert C, Mannioui A, Delache B, Brochard P, Guigand L, Dubreil L, Lebon P, Verrier B, et al., “Chikungunya disease in nonhuman primates involves long-term viral persistence in macrophages”, J Clin Invest, 2010, 120, pp. 894-906). To explore whether the E2EP3 epitope is a main target for the protective response, plasma samples from CHIKV-infected NHP were characterized with regard to their reactivity against E2EP3. Nine days after CHIKV-infection, plasma samples had already detectable anti-CHIKV IgG titers and importantly, also detected E2EP3 (FIG. 11(a)). FIG. 11(a) shows. E2EP3 specific antibodies titers in plasma samples (0, 9 and 13 days pi) being determined by E2EP3 specific peptide-based ELISA at a dilution of 1:2,000. Data were presented as mean f SD.

In in vitro neutralization assays CHIKV-infected NHPs plasma reduced CHIKV infectivity by 80% (FIG. 11(b)). In FIG. 11(b), anti-E2EP3 antibodies in CHIKV-infected NHP plasma were specifically blocked by soluble E2EP3 peptide, and followed by in vitro neutralization assay. Outputs were expressed as percentage infection relative to 0 dpi. Data were presented as mean±SD. A set of serial dilutions from 1:100 to 1:3,200 was made and samples assayed were performed in triplicates. * represents P<0.05; ** represents P<0.01; *** represents P<0.001 by two-way ANOVA with Bonferroni's multiple comparisons test.

Addition of soluble E2EP3 peptide abrogated the inhibitory effect of monkey plasma samples significantly throughout the whole dilution series (from 1:100 to 1:3200) when compared to the untreated plasma samples (FIG. 11(b)). Thus, as in humans, E2EP3 antibodies are part of the protective CHIKV response in NHPs.

The potential of E2EP3 epitope as a vaccine target was further assessed in a mouse model. For this, C57BL/6 mice were vaccinated with E2EP3 covalently linked to KLH in the presence of Freund's Adjuvant. Mice were primed and boosted twice with the immunogen (emulsified first with Complete [CFA] and then with Incomplete Freund's Adjuvant [IFA]) over a period of 21 days.

Non-human primate has humoral response to CHIKV similar to that of the human.

In effort to assess whether E2EP3 epitope may be a potential candidate for epitope vaccine design, the antigenicity was tested in relevant animal models.

Mice sera recognise B cell epitope of interest after rechallenge with CHKV particles.

BAL/C mice inoculated with CHIKV, and a booster shot of CHIKV particles was performed at Day 62 post-infection. Sera from mice were collected at day zero, 14, 21, 32, 62 and 75 post-infection (dpi) and used to detect mouse IgG against E2EP3 or more specifically, EMCp3 (FIG. 11(c)). After the first inoculation, antibodies to EMCp3 were detected after infection and peaked about day 21. A drop in antibody binding was observed in day 32 but antibody responses were restored after CHIKV re-infection (as indicated by the arrow in FIG. 11(c) at day 62).

Data from mice models confirmed that this epitope region is well-recognised across species, providing a good pre-clinical model for vaccine trials.

### Vaccination Schedule 2 for Longer CHIKV E2-KLH Peptide:

### Materials for Vaccination Schedule 2

3-week old C57BL/6 mice (7 mice per group)

Phosphate Buffered Saline (PBS)

Peptide: KLH—{STKDNFNVYKATRPYLAHC}

Adjuvants: (1) complete (only for first round) and incomplete (for subsequent rounds of vaccination) Freund's Adjuvant; (2) PAM3-Cys Adjuvant

### Groups

Group A: seven B6 mice/group>Peptide+CFA/IFA

Group B: seven B6 mice/group>PBS+CFA/IFA

Group C: seven B6 mice/group>Peptide+PAM3-Cys

Group D: seven B6 mice/group>PBD+PAM3-Cys

### Vaccination Method

Subcutaneous

100 μg of peptide for first injection

50 μg of peptide for subsequent injections

Start date for SGP011 challenge: 17 Jun. 2011 (Friday)

FIG. 12 shows a timeline representation of the SGP011 challenge.

### Followup Procedures

Bleed 1 (Day 15): (a) Peptide-based (KLH) ELISA

Bleed 2 (Day 22): (a) Peptide-based (KLH) ELISA; (b) Virion-based ELISA; and (c) In vitro neutralisation

Post Infection Follow-up (Day 23): (a) Footpad measurement (Day 23-Day 37); and (b) Plaque Assay (Day 25, Day 27 and Day 29).

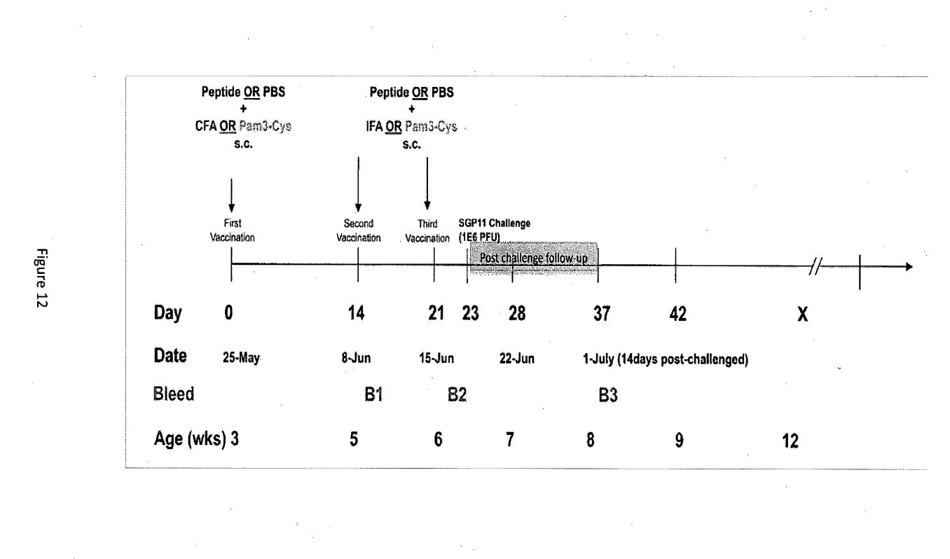

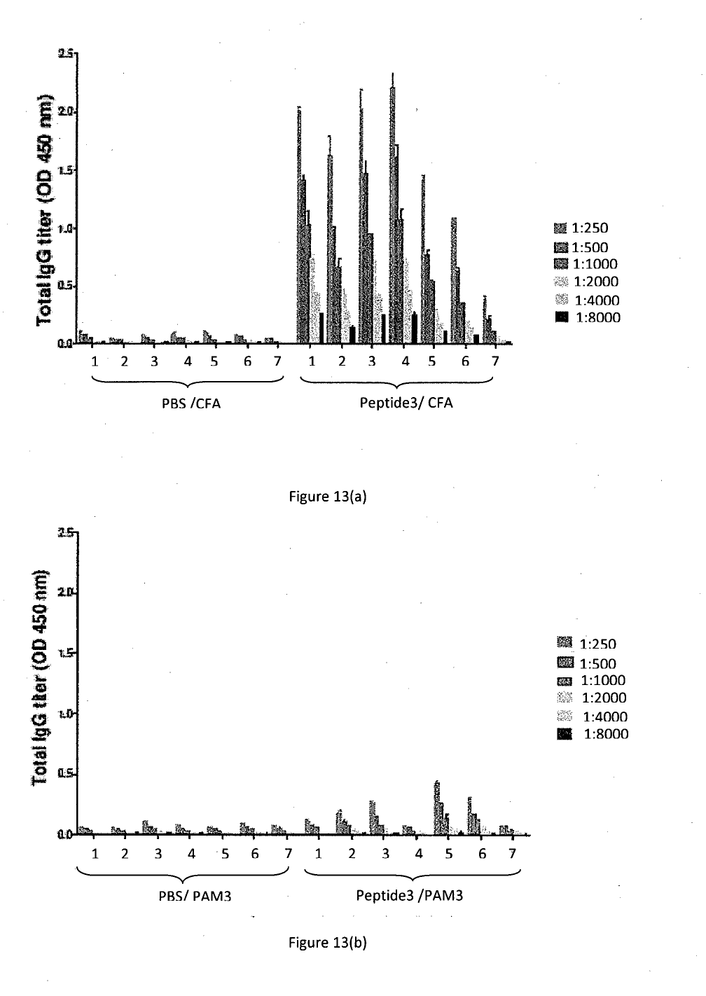

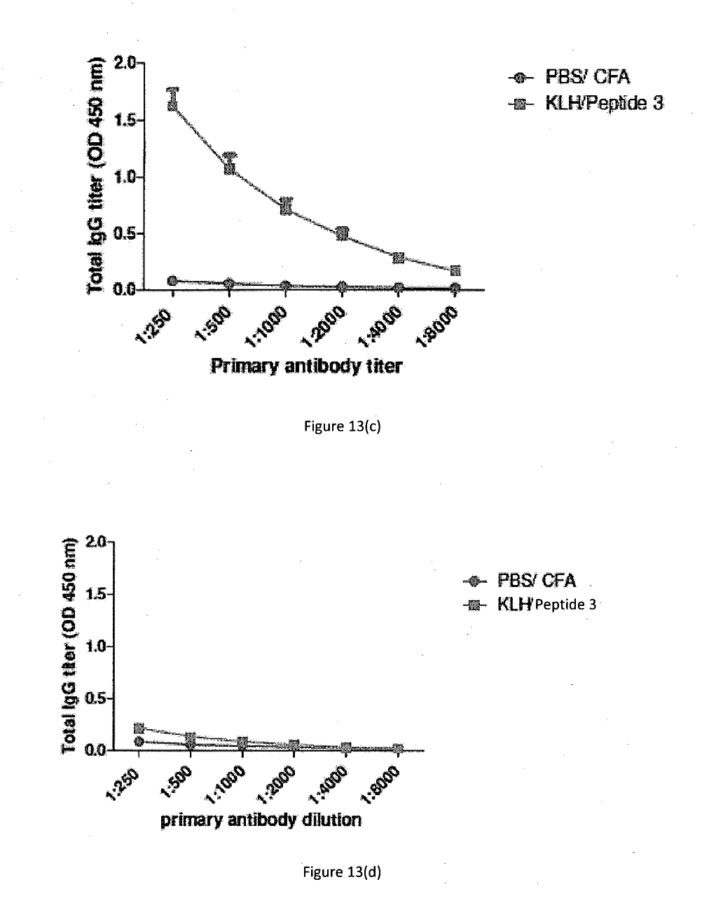

FIG. 13 shows titer of IgG against KLH-peptides from individual mice with (a) CFA-adjuvanted and (b) PAM3-adjuvanted for Bleed 1. Dilution factors of 1:250, 1:500, 1:1000, 1:2000, 1:4000, 1:8000 were conducted.

Peptide 3/CFA samples (1 to 7) shows larger total IgG titer than that of PBS/CFA samples (1 to 7), i.e, about 10 times larger.

Peptide 3/PAM3 samples (1 to 7) shows larger total IgG titer than that of PBS/CFA samples (1 to 7), i.e, about 3 times larger

FIG. 13 shows average titer of IgG against KLH-peptides for (c) CFA-adjuvanted group and (d) PAM3-adjuvanted group for Bleed 1. Dilution factors of 1:250, 1:500, 1:1000, 1:2000, 1:4000, 1:8000 were conducted. As the ratio increased from 1:250 to 1:8000, total IgG titer decreased correspondingly.

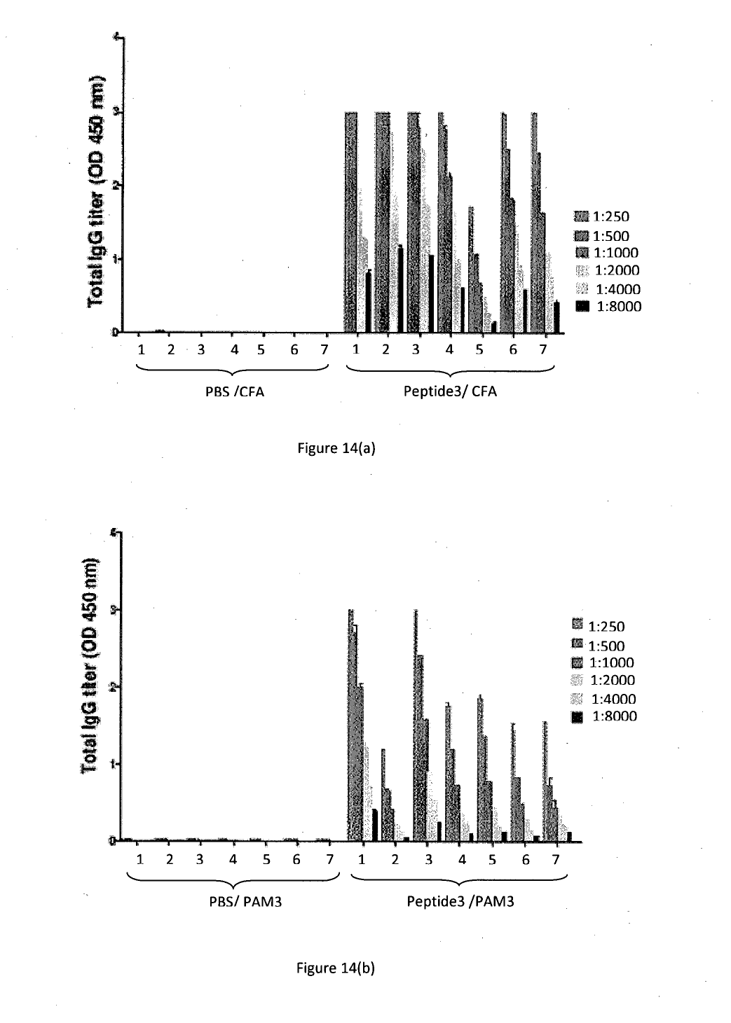

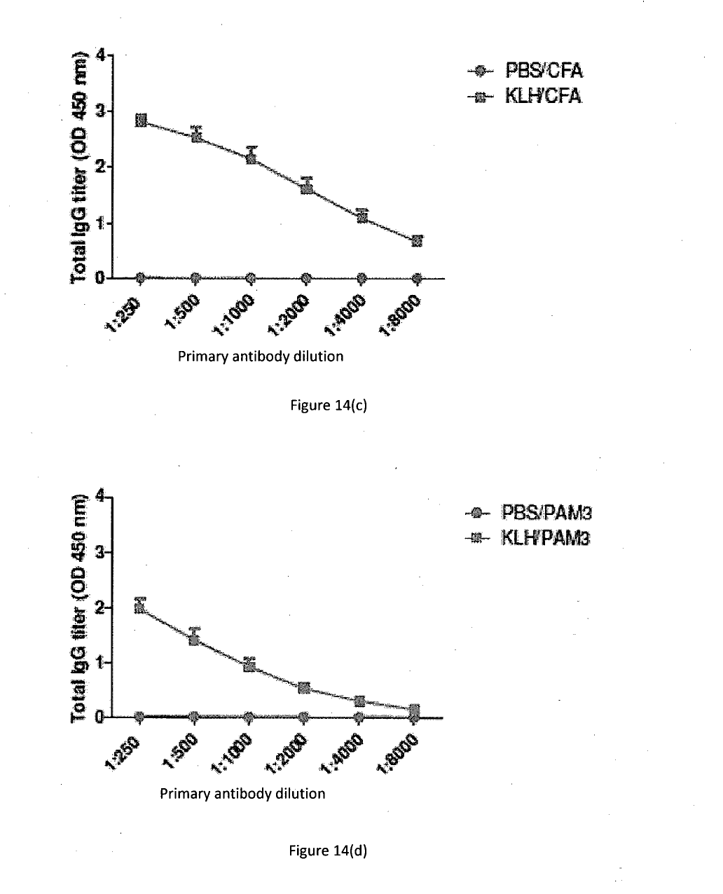

FIG. 14 shows titer of IgG against KLH-peptides from individual mice with (a) CFA-adjuvanted and (b) PAM3-adjuvanted for Bleed 2. Dilution factors of 1:250, 1:500, 1:1000, 1:2000, 1:4000, 1:8000 were conducted.

Peptide 3/CFA samples (1 to 7) shows larger total IgG titer than that of PBS/CFA samples (1 to 7), levels of which are almost zero.

Peptide 3/PAM3 samples (1 to 7) shows larger total IgG titer than that of PBS/CFA samples (1 to 7), levels of which are almost zero.

FIG. 14 shows average titer of IgG against KLH-peptides for (c) CFA-adjuvanted group and (d) PAM3-adjuvanted group for Bleed 2. Dilution factors of 1:250, 1:500, 1:1000, 1:2000, 1:4000, 1:8000 were conducted. As the ratio increased from 1:250 to 1:8000, total IgG titer decreased correspondingly.

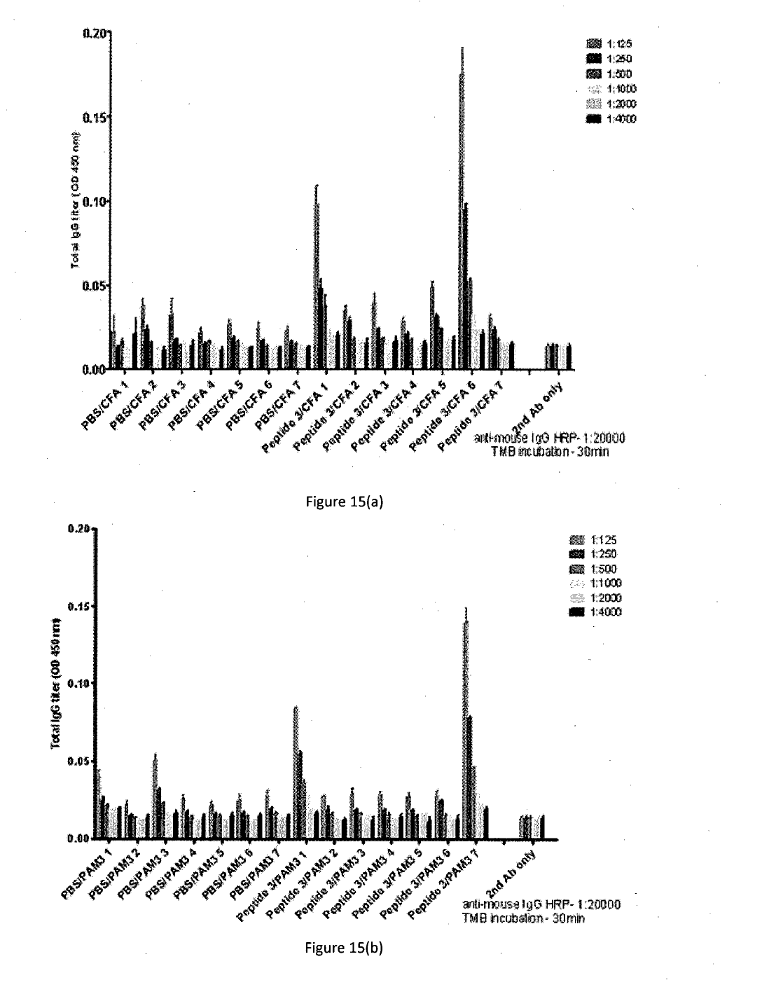

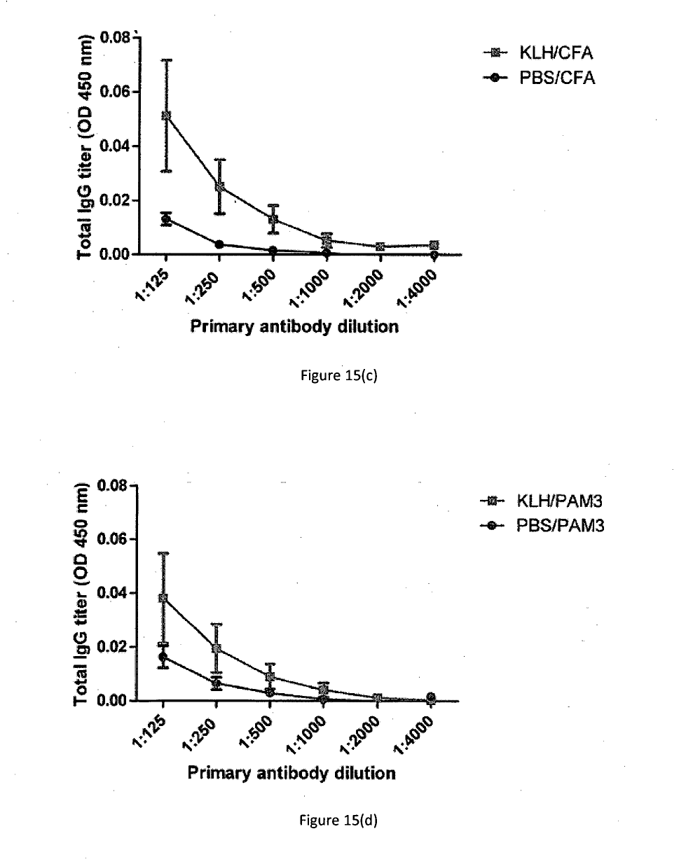

FIG. 15 shows titer of IgG against SGP11 virion from individual mice with (a) CFA-adjuvanted and (b) PAM3-adjuvanted for Bleed 2. Dilution factors of 1:125, 1:250, 1:500, 1:1000, 1:2000, 1:4000 were conducted.

Peptide 3/CFA samples (1 to 7) shows comparable total IgG titer than that of PBS/CFA samples (1 to 7), except for Peptide 3/CFA 1 and for Peptide 3/CFA 6 showing a surge increase in IgG titer, especially for dilution factors of 1:125, 1:250 and 1:500.

Peptide 3/PAM3 samples (1 to 7) shows comparable total IgG titer than that of PBS/CFA samples (1 to 7), except for Peptide 3/CFA 1 and for Peptide 3/CFA 1 showing a considerable increase in IgG titer, especially for dilution factors of 1:125, 1:250 and 1:500.

FIG. 15 shows average titer of IgG against SGP11 virion from individual mice with (c) CFA-adjuvanted and (d) PAM3-adjuvanted for Bleed 2. Dilution factors of 1:125, 1:250, 1:500, 1:1000, 1:2000, 1:4000 were conducted. Background signal were removed. As the ratio increased from 1:125 to 1:4000, total IgG titer decreased correspondingly.

KLH/CFA vaccinated group showed anti-CHIKV IgG antibodies response, up to 1:500 dilution. However, for KLH/PAM3 vaccinated group, the positive signal (or response) was not promising or indicative.

Measurement of inflamed footpad after virus challenge was performed. Mice were challenged on day 23 after the first vaccination and footpad was measured daily till 14 days after post infection (pi) challenged.

In a normal in vivo CHIKV infection in C57.BL6 mice, viremia peaked at 2 day post infection and viremia may fall below detection limit of plaque assay by day 5 post infection. Footpad may have two phases of inflammation namely primary peak on day 6 post infection and secondary peak on day 2 post infection.

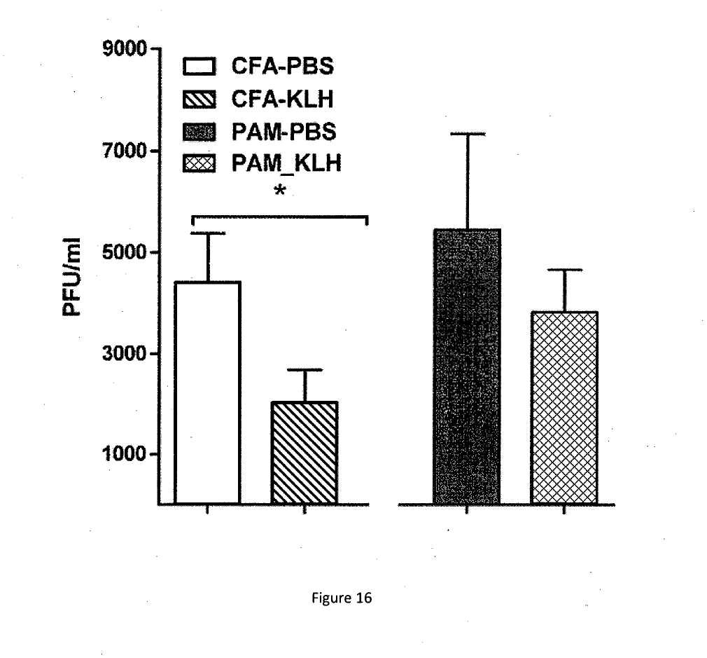

FIG. 16 shows a graph representing viremia on day 2 post infection. The sensitivity of the assay was 1000 PFU/ml of blood. Mann's Whitney analysis were used to compare CFA/PBS to CFA/KLH and PAM/PBS to PAM/KLH respectively (* represents p<0.05).

Viremia fell below detection limit of plaque assay for day 6 post infection. Plaque assay outcomes for day 4 post infection was found not suitable due to lifting of cells.

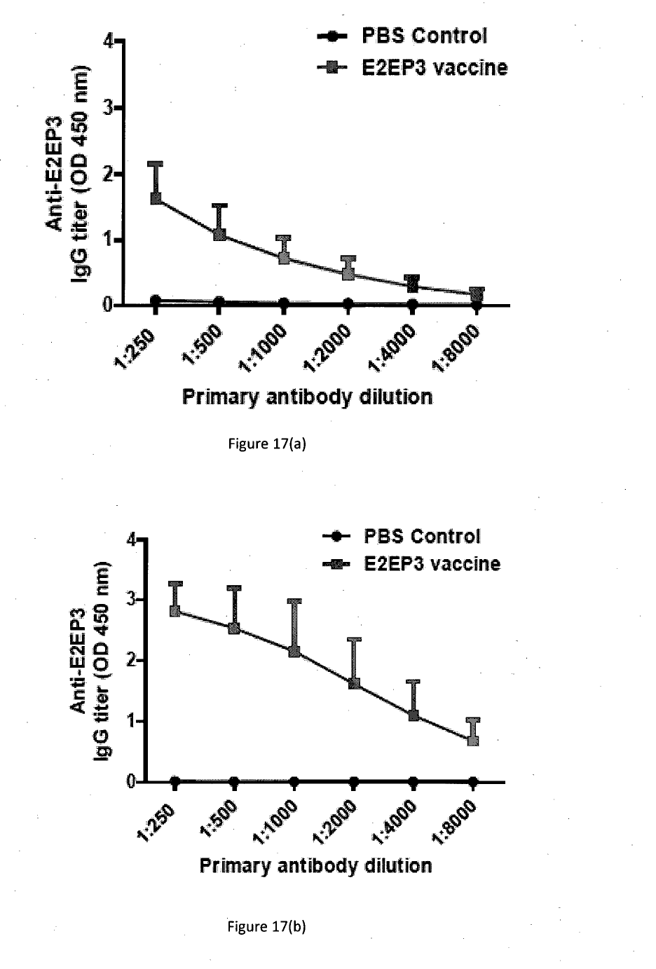

In FIGS. 17 to 20, the evaluation of protection against CHIKV challenge in mice vaccinated with E2EP3 peptide is provided.

Significant anti-E2EP3 titer was detected 19 days post-vaccination after the 1^{st} boost (FIG. 17(a)) and was further increased after the 2^{nd} boost at 27 days post-vaccination (FIG. 17(b)). Importantly, the sera obtained at 27 days post-vaccination were able to neutralize CHIKV-infection in vitro. In FIGS. 17(a) and 17(b), mouse anti-E2EP3 IgG antibodies were detected after the second and third vaccination. All outputs were expressed as mean±SD. Samples assayed were performed at triplicate.

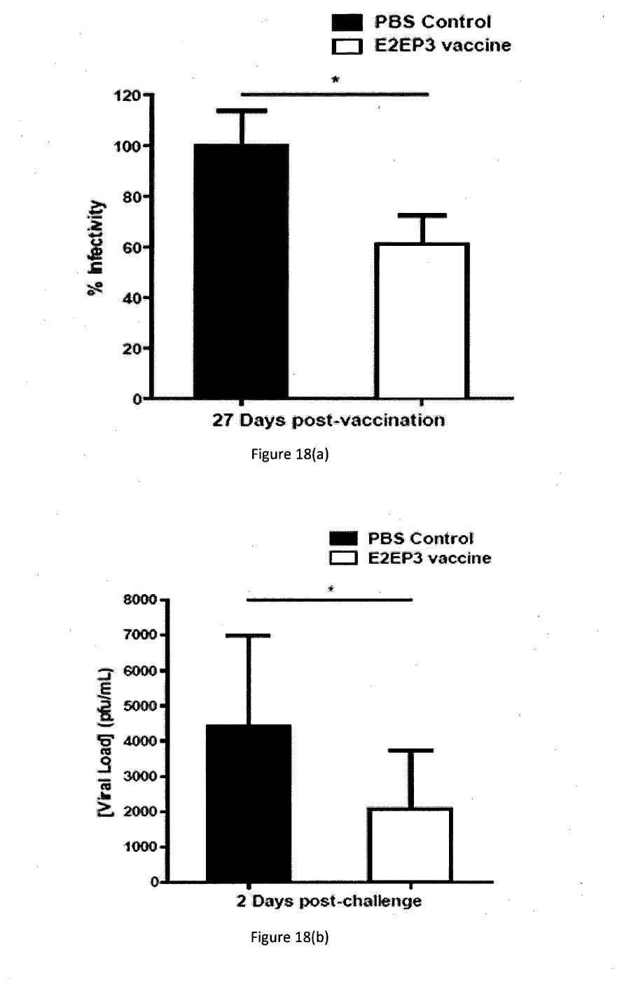

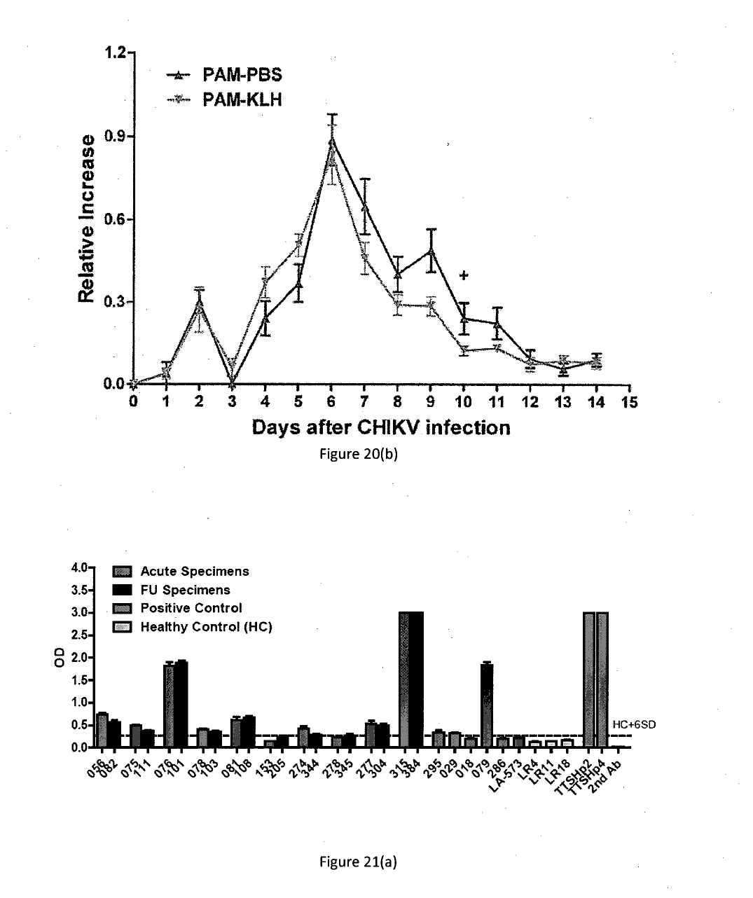

FIG. 18(a) shows in vitro neutralizing activity of E2EP3-vaccinated mouse sera. Mice were immunized with E2EP3 peptide complex to KLH or PBS Control emulsified with Complete Freund's Adjuvant (CFA) subcutaneously, and were boosted two more times with Incomplete Freund's Adjuvant (IFA). Sera was collected at 27 days post-vaccination and assayed for in vitro neutralization at a dilution of 1:100 (n=3). Outputs were expressed as percentage infection relative to PBS Control. Data were presented as mean±SD. * represents P<0.05 by Mann-Whitney U test. FIG. 18(b) shows mice immunized with E2EP3 or PBS Control being challenged subcutaneously with 10⁶ PFU CHIKV (SGP11). CHIKV viremia was measured at 2 days post-challenge by virus plaque assay. The detection limit was 1,000 pfu/mL. Data were presented as mean±SD. * represents P<0.05 by Mann-Whitney U test. FIG. 20(a) shows disease score measurement. Footpad sizes from day 0 to day 14 post-challenge were quantified by [width×thickness]. Footpad swelling (inflammation) relative to day 0 was obtained with the formula: [(day x−day 0)÷day 0], where x represents footpad sizes from day 1 to day 14. Data were presented as mean±SD. * represents P<0.05 by Mann-Whitney U test.

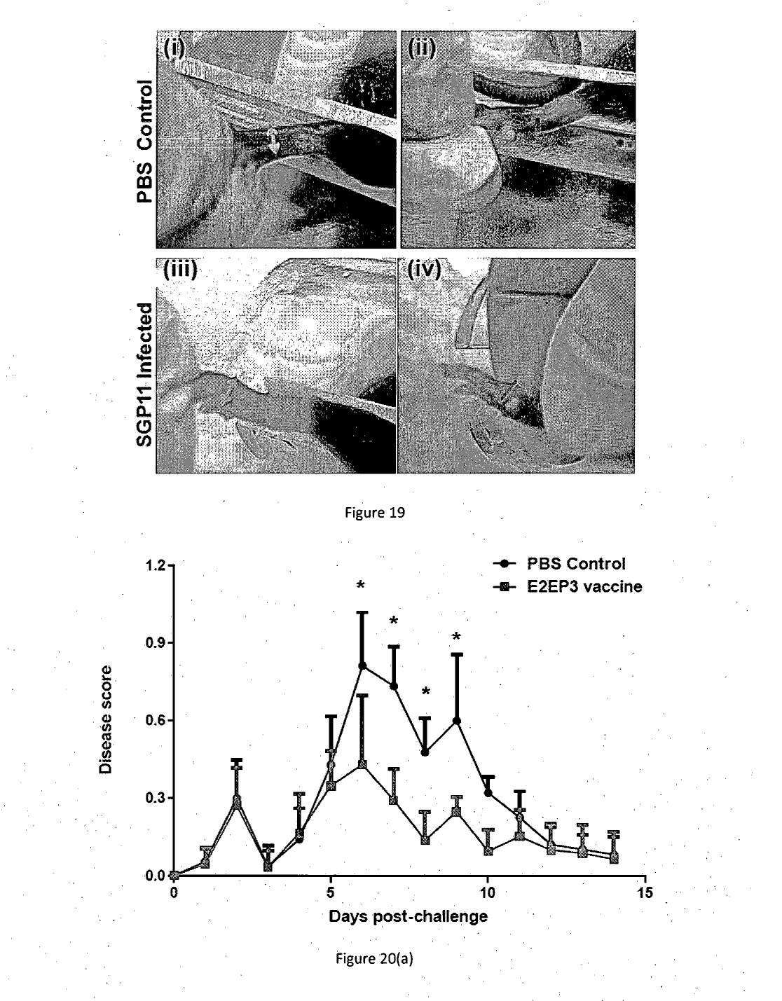

Compared to the PBS-vaccinated control group, infectivity was reduced by approximately 40% (FIG. 18(a)). Moreover, virus challenge in mice at 30 days post-vaccination indicated a partial protection by E2EP3 as viremia was reduced from 4,500 pfu/mL to 2,000 pfu/mL at 2 days post-challenge (FIG. 18(b)). This reduction of virus titer was also reflected in clinical symptoms used to monitor the virus-induced inflammation (FIG. 19).

FIG. 19 shows CHIKV-induced footpad inflammation. Effect of CHIKV injected into the footpad: (i) and (iii) represent respective photos of control and infected groups, and measurement of the width are indicated by double-headed arrows; and (ii) and (iv) represent respective photos of control and infected groups, and measurement of the thickness are indicated by lines.

Maximal footpad swelling in the PBS-vaccinated group was more than twice as that of the E2EP3-vaccinated group (FIG. 20(a)). E2EP3 may therefore be used both as a marker as well as a potential vaccine component in pre-clinical models for CHIKV therapy.

Measure of foot inflammation was also performed. FIG. 20(b) shows footpad sizes relative to day 0 for PAM group. Data were normalized to size relative to pre-infected phase (i.e., day 0). + represents p<0.05 for PAM group based on Mann's Whitney analysis. Footpad size increase peak about day 6 relative to day 0.

In a preliminary study on the naturally-acquired antibody response in CHIKV-infected patients, anti-CHIKV IgG were found to be detected only at the early convalescence phase of median 10 days pio. Typically, at that stage (i.e., early convalescence phase of median 10 days pio), most of the virus may already be cleared and may usually be no longer detectable in the blood. More surprisingly, virtually all anti-CHIKV IgG found at that stage of the disease were observed to be of the IgG3 isotype. While it may be expected that the early neutralizing antibody response was targeting the proteins of the envelope of the virus, it was shown in this example that in fact most of these IgG3 antibodies recognized a single epitope forming a prominently exposed stalk on the E2 glycoprotein.

When using complete CHIKV virion particles E2 glycoprotein was the only one of the three known surface proteins that reacted to the IgG of the patients' plasma. Neither capsid nor E1 glycoprotein were detectable by western blot analysis. It was shown that other structural proteins including the E1 glycoprotein (Cho B, Jeon B Y, Kim J, Noh J, Park M, Park S, “Expression and evaluation of Chikungunya virus E1 and E2 envelope proteins for serodiagnosis of Chikungunya virus infection”, Yonsei Med J, 2008, 49, pp. 828-835; Kowalzik S, Xuan N V, Weissbrich B, Scheiner B, Schied T, Drosten C, Muller A, Stich A, Rethwilm A, Bodem J, “Characterisation of a chikungunya virus from a German patient returning from Mauritius and development of a serological test”, Med Microbiol Immunol, 2008, 197, pp. 381-386; Yap G, Pok K Y, Lai Y L, Hapuarachchi H C, Chow A, Leo Y S, Tan L K, Ng L C, “Evaluation of Chikungunya diagnostic assays: differences in sensitivity of serology assays in two independent outbreaks” PLoS Negl Trop Dis, 2010, 4, e753) and capsid (Cho B, Kim J, Cho J E, Jeon B Y, Park S, “Expression of the capsid protein of Chikungunya virus in a baculovirus for serodiagnosis of Chikungunya disease”, J Virol Methods, 2008, 154, pp. 154-159) were also detected to varying degrees by patients' IgGs from patients' samples collected at later time points or stages. However, especially at the early phase of infection the E2 glycoprotein was apparently the only major target. At later time points, contributions by epitopes of other proteins may further increase the complexity of the patterns of antigenic recognition.

CHIKV represents a ‘novel’ virus for the naive population. Most infected individuals did not have any prior encounters with CHIKV, and therefore lacked the complete CHIKV-specific antibodies. E2EP3 may be an early target since it is a structural element shared with other alphaviruses.

While E2 glycoprotein was clearly the dominant surface antigen, the most striking observation was that a vast majority of the early anti-CHIKV IgG3 antibodies were directed against a single linear epitope. Depletion examples indicated that E2EP3-specific antibodies represented nearly 70 to 80% of the anti-CHIKV IgG of the patients' sera (FIG. 9(c)). Published crystal structure data and alanine scan revealed the precise location of this dominant epitope.

E2EP3 is located at the N-terminus of the E2 glycoprotein. It is part of the furin-loop and forms a prominent little stalk facing away from the virus envelope with sufficient flexibility for antibody recognition. While it almost appears to be ‘destined’ to be recognized by antibodies, its surface exposure is likely to be a consequence of the need to be reached by furin. Furin is a golgi-resident protease (Thomas G, “Furin at the cutting edge: from protein traffic to embryogenesis and disease”, Nat Rev Mol Cell Biol, 2002, 3, pp. 753-766) and is also used by various viruses including HIV (Hallenberger S, Bosch V, Angliker H, Shaw E, Klenk H D, Garten W, “Inhibition of furin-mediated cleavage activation of HIV-1 glycoprotein gp160”, Nature, 1992, 360, pp. 358-361). It is mandatory for the maturation of alphaviruses where it facilitates cleavage of the p62 precursor into E2 and E3 glycoproteins (Heidner H W, Knott T A, Johnston R E, “Differential processing of sindbis virus glycoprotein PE2 in cultured vertebrate and arthropod cells: J Virol, 1996, 70, pp. 2069-2073; Zhang X, Fugere M, Day R, Kielian M, “Furin processing and proteolytic activation of Semliki Forest virus”, J Virol, 2003, 77, pp. 2981-2989; Ozden S, Lucas-Hourani M, Ceccaldi P E, Basak A, Valentine M, Benjannet S, Hamelin J, Jacob Y, Mamchaoui K, Mouly V, et al., “Inhibition of Chikungunya virus infection in cultured human muscle cells by furin inhibitors: impairment of the maturation of the E2 surface glycoprotein”, J Biol Chem, 2008, 283, pp. 21899-21908.

Early anti-CHIKV IgG3 were strongly neutralizing. In this study, these findings were extended verifying that E2EP3-specific antibodies were able to block viral infection (FIG. 9). Examples further showed that neutralizing antibodies to this epitope were also present in plasma samples of NHPs (FIG. 11). Thus, E2EP3 was shown to be important for viral defense both in humans as well as in the pre-clinical animal model commonly used for the study of CHIKV infections.

Notably, E2EP3 is a true linear determinant. In mice, it may therefore be shown that short E2EP3 peptides linked to KLH may indeed be able to induce protective antibody responses. E2EP3 may therefore represent an ideal candidate that could be incorporated in vaccine formulations aiming to prevent CHIKV infections. As a basic proof-of-principle, it was shown in the mouse model that a simple peptide formulation was effective at inducing neutralizing antibodies that not only reduced viremia, but also diminished viral induced-pathologies such as joint inflammation (FIG. 17).

Antibodies to E2EP3 were detected during early convalescence after viremia was cleared. These antibodies served as reliable early serologic markers for CHIKV infections. In three independent cohorts (2 from Singapore and 1 from Malaysia), E2EP3-specific antibodies were detected in almost all the blood samples taken between 10 to 14 median days pio from infected patients, whereas none of the control plasma reacted against the epitope. E2EP3 may therefore be used in diagnostic kits, such as epitope-based immunochromatographic tests (ICT). Early detection may allow for more cost-effective patient management (Cuzzubbo A J, Endy T P, Nisalak A, Kalayanarooj S, Vaughn D W, Ogata S A, Clements D E, Devine P L, “Use of recombinant envelope proteins for serological diagnosis of Dengue virus infection in an immunochromatographic assay”, Clin Diagn Lab Immunol, 2001, 8, pp. 1150-1155; Marot-Leblond A, Nail-Billaud S, Pilon F, Beucher B, Poulain D, Robert R, “Efficient diagnosis of vulvovaginal candidiasis by use of a new rapid immunochromatography test”, J Clin Microbiol, 2009, 47, pp. 3821-3825) since high levels of IgG3 at that time may be associated with an absence of persistent arthralgia. In addition, E2EP3 may also be used for serology detection in sylvatic infections of primates just like screening of SIVs-infected animals with peptides in Africa (Simon F, Souquiere S, Damond F, Kfutwah A, Makuwa M, Leroy E, Rouquet P, Berthier J L, Rigoulet J, Lecu A, et al., “Synthetic peptide strategy for the detection of and discrimination among highly divergent primate lentiviruses”, AIDS Res Hum Retroviruses, 2001, 17, pp. 937-952; Worobey M, Telfer P, Souquiere S, Hunter M, Coleman C A, Metzger M J, Reed P, Makuwa M, Hearn G, Honarvar S, et al., “Island biogeography reveals the deep history of SIV”, Science, 2010, 329, pp. 1487).

Notably, patients who rapidly developed high levels of IgG3 has higher viremia and endured a more severe disease during the acute viremic phase, but did not experience persistent arthralgia. Thus, the early induction of IgG3 antibodies is a marker of protection against persistent arthralgia.

It may allow identification of patients with increased risks of disease and may imply that low viral load during acute infection may compromise establishing fully protective immunity.

N-terminal portion (aa 1-19) of the E2 glycoprotein was found to represent one of the targets of anti-CHIKV IgG3. Sequence of the peptide region called E2EP3 is STKDNFNVYKATRPYLAHC (SEQ ID No. 89). As a linear B-cell epitope, it may have potential use in future diagnostics and therapeutic applications.

The peptide-based screen was sensitive enough to detect specific epitopes that recognise the CHIKV E2 glycoprotein. Although the signals differ for the peptides, this may be due to the different binding affinities of the CHIKV antibodies and the epitope regions. Other influencing factors may be due to the different degree of exposure of the amino acid residue were on the glycoprotein. Steric hinderance as well as chemical properties of the epitopes which may in turn affect the chemical bonds between the antibody and the epitopes may be another factor. Nonetheless, the epitope regions identified in this example have been verified directly from patients and may act good targets for diagnostic markers and vaccine candidates.

In summary, it was established that the naturally-acquired early IgG3 response against CHIKV was strongly focused on the E2EP3 epitope. As a simple linear epitope, it may open new options for both diagnostic and prevention of CHIKV infections. Due to the resurgence of CHIKV and other alphaviruses, interests for prophylactic vaccines have already regained importance. Such vaccines would be useful for travelers and/or populations at risk during outbreaks and E2EP3 could become an integral component to achieve protection.

### Screening of CHIKV Antibodies (IgG and IgM) Against SGP11 Virion in 16 Thailand Patients Samples:

### Materials for Screening of Thailand Patients Samples

SGP11 Virion Coated Plate

Constituted peptide 3

Patients' Samples (batch 1)

TABLE 2
Samples ID

| | | | |
|---|---|---|---|
| 08P00056 | 08P00082 | 08P00278 | 08P00345 |
| 08P00075 | 08P00111 | 08P00227 | 08P00304 |
| 08P00076 | 08P00101 | 08P00315 | 08P00384 |
| 08P00078 | 08P00103 | 08P00295 | 08P00279 |
| 08P00081 | 08P00108 | 08P00029 | 08P00286 |
| 08P00153 | 08P00205 | 08P00018 | LAB-00573 |
| 08P00274 | 08P00344 | | |

Materials for Virion based-ELISA:
Coating buffer (PBS)
Washing buffer (PBST) (PBS+0.05% Tween 20)
Blocking buffer (PBST+5% milk)
Blocking buffer for antibodies (PBST+2.5% milk)
Maxisorp 96-well plate (Nunc 44-2404) (from storeroom)

Materials for Peptide 3-ELISA:
1×PBS: 0.01 M, pH 7.2
Washing buffer: 0.1% PBST (1×PBS supplied with 0.1% v/v Tween 20)
Blocking buffer: 0.1% PBST supplied with 1% w/v sodium caseinate (Sigma-Aldrich cat #C8654)
Conjugate diluent: 0.1% PBST+0.1% w/v sodium caseinate
Secondary antibody: HRP-conjugated goat anti-human IgG (H+L) (Invitrogen cat #62-7120)
Substrate solution: TMB (Sigma-Aldrich cat #T8665)
Stop solution: 0.5 M H₂SO₄ (Sigma-Aldrich cat #S5814)
Streptavidin-coated plate (clear, 96-well, from Pierce #15124)

### Methods for Screening of Thailand Patients Samples

Preparation of SGP11 coated Plates (10 plates):
Prepare purified CHIKV (SGP011, sucrose cushion purified, by Fok Moon) (1.85e9 copies/ul). Dilute to 2000 virion/μl
Dispense 50 ul into each well of the plate.
Cover the plate, rock for about 1 day in about 4° C. and store plate at about 4° C. Plates kept longer than 2 months from preparation were discarded.

Detection Virion-based ELISA:
Remove the coating solution. Wash the plate 6 times with washing buffer.
Fill the wells (300 μl/well) with blocking buffer.
Incubate for about 1.5 hours at about 37° C. (in CO₂ incubator).
Wash plate 6 times with PBST.
Dilute patient plasma by 2000× in 1 ml of milk/PBST. Add 100 μl of 1^{st} antibody (diluted plasma) in blocking buffer into the appropriate wells.
Cover the plate and incubate for about 1 hour at about 37° C. (in CO₂ incubator).
Wash 6 times with PBST.
Dilute anti human IgG or IgM antibodies 4000× in milk/PBST. Add 100 μl of 2^{nd} antibody in blocking buffer into the appropriate wells.
Cover the plate and incubate for about 0.5 hour at about 37° C. (in CO₂ incubator).
Wash 6 times with PBST
Add 100 μl of 1×TMB to each well.
Incubate for about 15 minutes (IgG) and about 30 minutes (IgM) at room temperature in the dark.
Add 100 μl stop solution to each well.
Read plate at 450 nm.

Detection Peptide 3 ELISA:
Block non-specific absorption by dispensing 200 μA of blocking buffer into each well of the dry, streptavidin-coated plate. Allow to incubate for about 1 hour at about 20° C.
Wash the plates with PBST, 4 times.
Peptide 3 solutions are diluted to a working strength of 1:1000 with PBST.
Transfer 100 μl of each of the diluted peptide solutions into the corresponding well positions of the streptavidin-coated plate.
Place the plate on a shaker table and allow the reaction to proceed for about 1 hour at room temperature. After incubation, wash plate 5× with PBST. [2 plates was air dried in room temperature, sealed and are further tested at 2 weeks and 1 month time point.]
Dilute the serum to be tested, using conjugate diluent. For total IgG samples, serum were diluted 1:2000. For IgG₃ samples, sera were diluted 1:1000. Add 100 μl of the diluted serum to each of the wells of the plates containing captured peptides. Place the plate on a shaker table and incubate with agitation for about 1 hour at about 20° C.
Remove the incubation mixture, wash 5× with PBST. Detect bound antibody with a suitable dilution of conjugate solution consisting of a saturating level of horse radish peroxidase-labelled anti-species antibody. For total IgG samples, antibody is diluted 1:4000. For IgG₃, antibody were diluted 1:500. Dispense 100 μl of the dilute conjugate into each well and incubate at about 20° C. for about 1 hour with agitation.
Remove the incubation mixture by flicking the plate and repeat the washes as previously described. Finally, wash the plate twice with PBS only.
Detect the presence of peroxidase by adding 100 μl of TMB substrate solution to each well. Total IgG samples were incubated for about 10 minutes. IgG₃ samples were incubated for about 45 minutes. Add 100 μl of Stop reagent per well and measure absorbance (OD) using a microplate reader at 450 nm (reference wavelength approx. 690 nm). IgG3 detection was repeated at 1:200 dilution for primary antibody with 10-minute TMB step.

### Plates Layout

TABLE 3
Anti-IgM secondary antibody

| Plate 1 | 1 | 2 | 3 | 4 | 5 | 6 | 7 | 8 | 9 | 10 | 11 | 12 |
|---|---|---|---|---|---|---|---|---|---|---|---|---|
| A | 056 | 056 | 056 | 082 | 082 | 082 | 075 | 075 | 075 | 111 | 111 | 111 |
| B | 076 | 076 | 076 | 101 | 101 | 101 | 078 | 078 | 078 | 103 | 103 | 103 |
| C | 081 | 081 | 081 | 108 | 108 | 108 | 153 | 153 | 153 | 205 | 205 | 205 |
| D | 274 | 274 | 274 | 344 | 344 | 344 | 278 | 278 | 278 | 345 | 345 | 345 |
| E | 227 | 227 | 227 | 304 | 304 | 304 | 315 | 315 | 315 | 384 | 384 | 384 |
| F | 295 | 295 | 295 | 029 | 029 | 029 | 018 | 018 | 018 | 279 | 279 | 279 |
| G | 286 | 286 | 286 | LA-573 | LA-573 | LA-573 | LR4 | LR4 | LR4 | LR11 | LR11 | LR11 |
| H | LR18 | LR18 | LR18 | TTSHp2 | TTSHp2 | TTSHp2 | TTSHp4 | TTSHp4 | TTSHp4 | 2nd | 2nd | 2nd |
| | | | | | | | | | | AB | AB | AB |

TABLE 4
Anti-IgG secondary antibody

| Plate 1 | 1 | 2 | 3 | 4 | 5 | 6 | 7 | 8 | 9 | 10 | 11 | 12 |
|---|---|---|---|---|---|---|---|---|---|---|---|---|
| A | 056 | 056 | 056 | 082 | 082 | 082 | 075 | 075 | 075 | 111 | 111 | 111 |
| B | 076 | 076 | 076 | 101 | 101 | 101 | 078 | 078 | 078 | 103 | 103 | 103 |
| C | 081 | 081 | 081 | 108 | 108 | 108 | 153 | 153 | 153 | 205 | 205 | 205 |
| D | 274 | 274 | 274 | 344 | 344 | 344 | 278 | 278 | 278 | 345 | 345 | 345 |
| E | 227 | 227 | 227 | 304 | 304 | 304 | 315 | 315 | 315 | 384 | 384 | 384 |
| F | 295 | 295 | 295 | 029 | 029 | 029 | 018 | 018 | 018 | 279 | 279 | 279 |
| G | 286 | 286 | 286 | LA-573 | LA-573 | LA-573 | LR4 | LR4 | LR4 | LR11 | LR11 | LR11 |
| H | LR18 | LR18 | LR18 | TTSHp2 | TTSHp2 | TTSHp2 | TTSHp4 | TTSHp4 | TTSHp4 | 2nd | 2nd | 2nd |
| | | | | | | | | | | AB | AB | AB |

### OD Readings Using Virion Base ELISA

Table 5 shows a summary of average IgG OD and average IgM OD measured for acute plasma samples and FU plasma samples, listed in Table 2.

TABLE 5

| | Avg | CHIKV | Avg | CHIKV | | Avg | CHIKV | Avg | CHIKV | |
|---|---|---|---|---|---|---|---|---|---|---|
| Acute | IgG | IgG | IgM | IgM | FU | IgG | IgG | IgM | IgM | |
| Plasma | OD | Positive | OD | Positive | plasma | OD | Positive | OD | Positive | Dengue |
| 08P00056 | 0.743 | Yes | 0.210 | No | 08P00082 | 0.553 | Yes | 0.206 | No | Yes |
| 08P00075 | 0.485 | Yes | 0.255 | No | 08P00111 | 0.373 | Yes | 0.230 | No | Yes |
| 08P00076 | 1.809 | Yes | 0.343 | No | 08P00101 | 1.888 | Yes | 0.483 | No | Yes |
| 08P00078 | 0.409 | Yes | 0.596 | No | 08P00103 | 0.353 | Yes | 0.498 | No | Yes |
| 08P00081 | 0.617 | Yes | 0.260 | No | 08P00108 | 0.663 | Yes | 0.279 | No | Yes |
| 08P00153 | 0.143 | No | 0.079 | No | 08P00205 | 0.239 | No | 0.121 | No | No |
| 08P00274 | 0.419 | Yes | 0.162 | No | 08P00344 | 0.282 | Yes | 0.144 | No | No |
| 08P00278 | 0.232 | No | 0.254 | No | 08P00345 | 0.275 | Yes | 0.301 | No | No |
| 08P00227 | 0.530 | Yes | 0.220 | No | 08P00304 | 0.488 | Yes | 0.246 | No | No |
| 08P00315 | 3.000 | Yes | 0.199 | No | 08P00384 | 3.000 | Yes | 0.274 | No | No |
| 08P00295 | 0.342 | Yes | 0.160 | No | | | | | | NA |
| 08P00029 | 0.314 | Yes | 0.177 | No | | | | | | NA |
| 08P00018 | 0.193 | No | 0.063 | No | | | | | | NA |
| 08P00279 | 1.833 | Yes | 0.204 | No | | | | | | NA |
| 08P00286 | 0.197 | No | 0.196 | No | | | | | | NA |
| LAB-00573 | 0.220 | No | 0.117 | No | | | | | | NA |

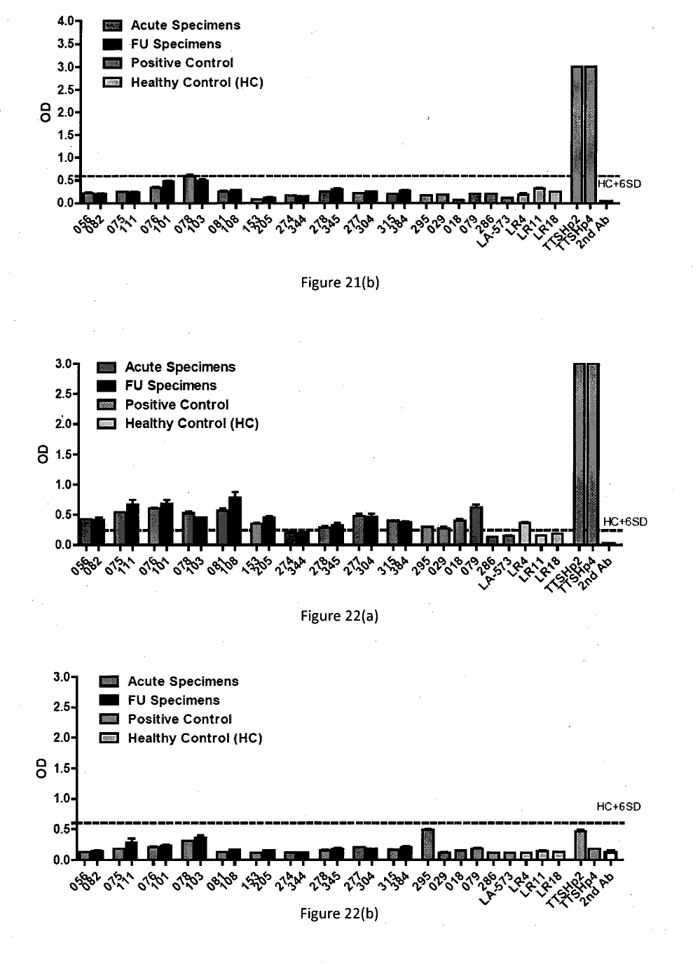

Patients were considered positive when OD reading was greater than average OD of (healthy controls+6SD) (FIGS. 21(a) and 21(b)).

FIG. 21(a) shows OD reading of IgG using virion base ELISA. LR4, LR11 and LR18 were used as healthy controls (samples were from previously screened negative patients from Thailand). Samples with OD reading beyond the ranged readable by machine was assigned a value of ‘3’. All samples were labeled as the last 3 digits of plasma ID given in Table 5. Cut off for positive readings was set at HC+6SD. Pooled serum samples from TTSH CHIKV patients at time point 2 and time point 4 were used as positive controls. The definitions for the samples are also applicable for FIG. 21(b).

FIG. 21(b) shows OD reading of IgM using virion base ELISA.

### OD Readings Using Peptide 3 ELISA

Table 6 shows a summary of virion IgG OD, average IgG OD and average IgG30D measured for acute plasma samples and FU plasma samples, listed in Table 2.

TABLE 6

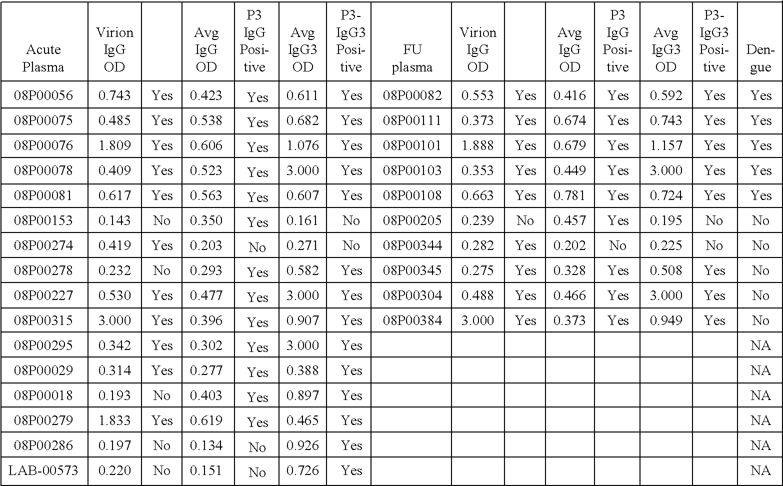

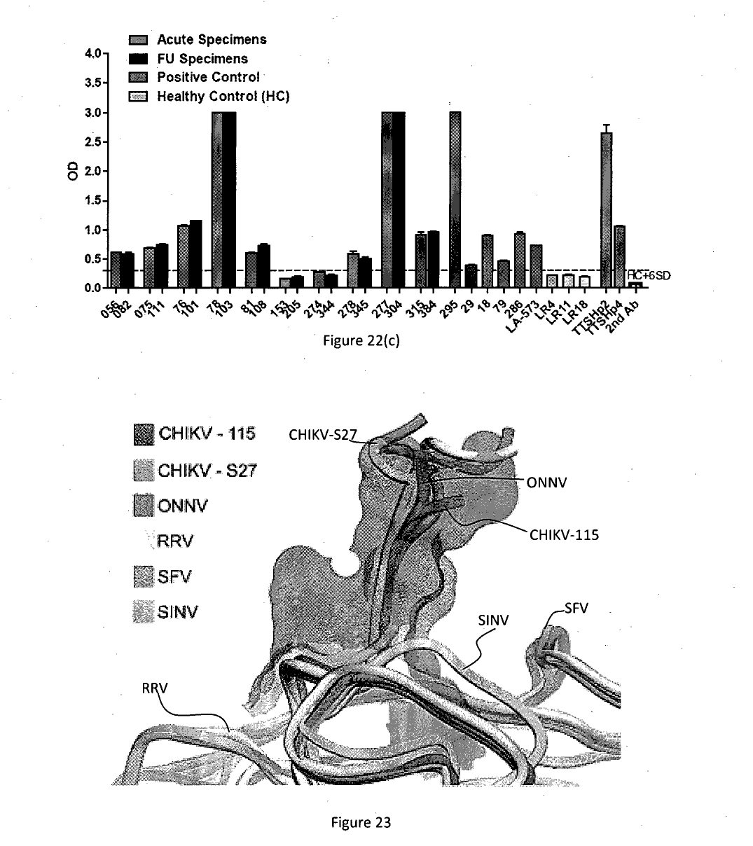

Patients were considered positive when OD reading was greater than average OD of (healthy controls+6SD) (FIGS. 22(a)-22(c)).

FIG. 22(a) shows OD reading of total IgG using E2EP3 peptide-based ELISA. LR4, LR11 and LR18 were used as healthy controls (samples were from previously screened negative patients from Thailand). For peptide 3 of total IgG, LR4 was removed due to high OD reading. Discrepancies with virion-based ELISA are as shaded in Table 6. Samples with OD reading beyond the ranged readable by machine was assigned a value of ‘3’. All samples were labeled as the last 3 digits of plasma ID given in Table 6. LR4 was excluded as the healthy control due to higher reading. Cut off for positive readings was set at HC+6SD. Pooled serum samples from TTSH CHIKV patients at time point 2 and time point 4 were used as positive controls. The definitions for the samples are also applicable for FIGS. 22(b) and 22(c) with LR4 included as the healthy control.

FIG. 22(b) shows OD reading of IgG3 using E2EP3 peptide-based ELISA (1 in 1000 patients serum dilution).

FIG. 22(c) shows OD reading of IgG3 using E2EP3 peptide-based ELISA (1 in 200 patients serum dilution).

From FIG. 21(a), the (average+6SD) total IgG for virion based ELISA was about 0.2593. From FIG. 22(a), the (average+6SD) total IgG for E2EP3 based ELISA was about 0.2247. From FIG. 22(c), the (average+6SD) IgG3 for E2EP3 based ELISA was about 0.3038.

In another independent cohort (from Thailand), E2EP3-specific IgG3 antibodies were detected in over 90% of the serum samples taken from patients during the acute phase of infection. Therefore, this study further validate the potential of E2EP3 specific IgG3 as a commone marker of CHIKV infection.

### E2EP3 Epitope Region is Conserved Across Other Important Alphaviruses:

Since E2EP3 epitope region is well-recognised across species an has the potential for pre-clinical vaccination trials, it was assessed whether if this epitope region may be further developed for other clinically important alphaviruses.

Sequence and structural analyses have indicated that this region is highly conserved in other alphaviruses such as O'nyong nyong virus (ONNV) found in Africa, Ross River virus (RRV) found in Australia, Semlili Forest virus (SFV) found in Europe and Sinbis virus (SV) (FIG. 23).

FIG. 24 shows a summary of exemplary algorithms. From these exemplary algorithms, BFE-SVM20 was shown to be the best classifer.

### Single Amino Acid Substitution in Peptides 350 and 351 (E2EP3) Resulted in Alteration of Antibody-Antigen Interactions:

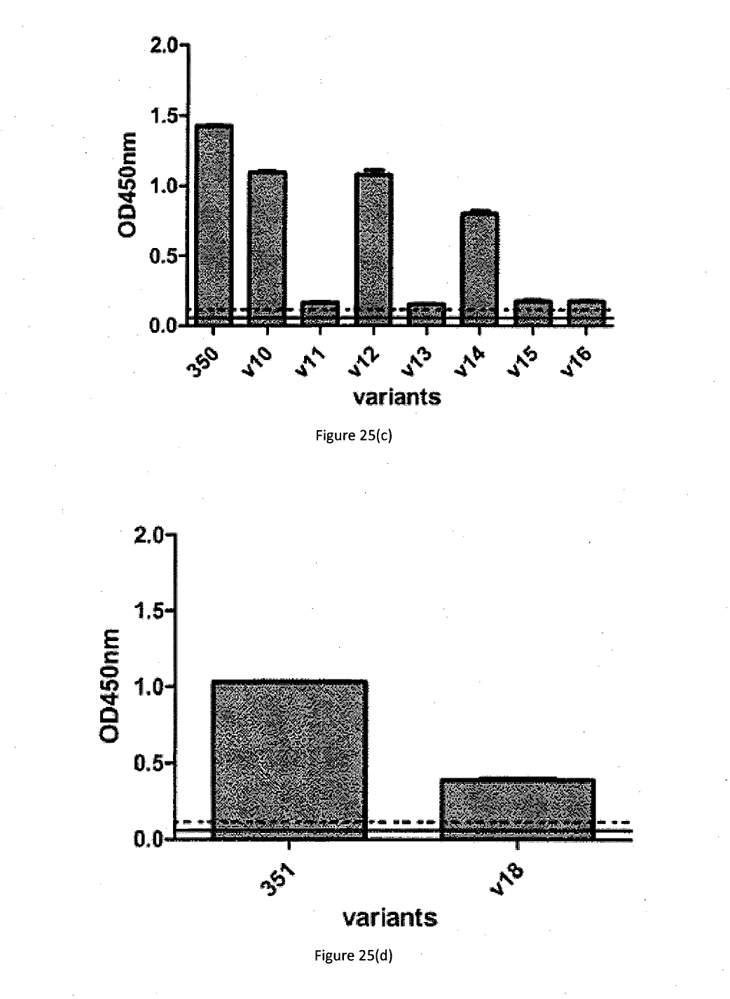

In an effort to look for amino acid variations across the different CHIKV isolates, residues that differ from the consensus sequence within this epitope region were synthesized as new peptides (denoted by v10-16). In several of the variants, there was a reduction in antibody binding ability. Intriguingly, this coincided with the change in residue from asparagine (N) to histine (H) in specific position (as indicated by respective arrowed boxed areas) in FIGS. 25(a) and 25(b). This phenomenon was observed in both peptides 350 (FIG. 25(c)) and 351 (FIG. 25(d)) as the residue of interest lied in the overlapping region between the two peptides.

### Key Amino Acid Residues Involved in E2EP3 Epitope Region:

A series of peptides were generated based on E2EP3 sequence in order to perform an alanine scan (alanine scan is able to identify specific amino acid residues responsible for a peptide's activity) study to identify key amino acid residues involve in the epitope region. Outputs from patients' plasma indicated that amino acid residue 3 and 10 are very important due to the lost of binding capacity, while amino acid residues 5 and 8 are important, and amino acid residue 9 is slightly important. Amino acid residue 3 was not resolved by the crystal structure.

The five important amino acid residues were located at the structural level of the E2 glycoprotein. It was observed that amino acid residue may be involved directly with Ab-binding, while amino acid residues 8, 9, and 10 may be involved in maintaining the structure of the epitope based on their positions.

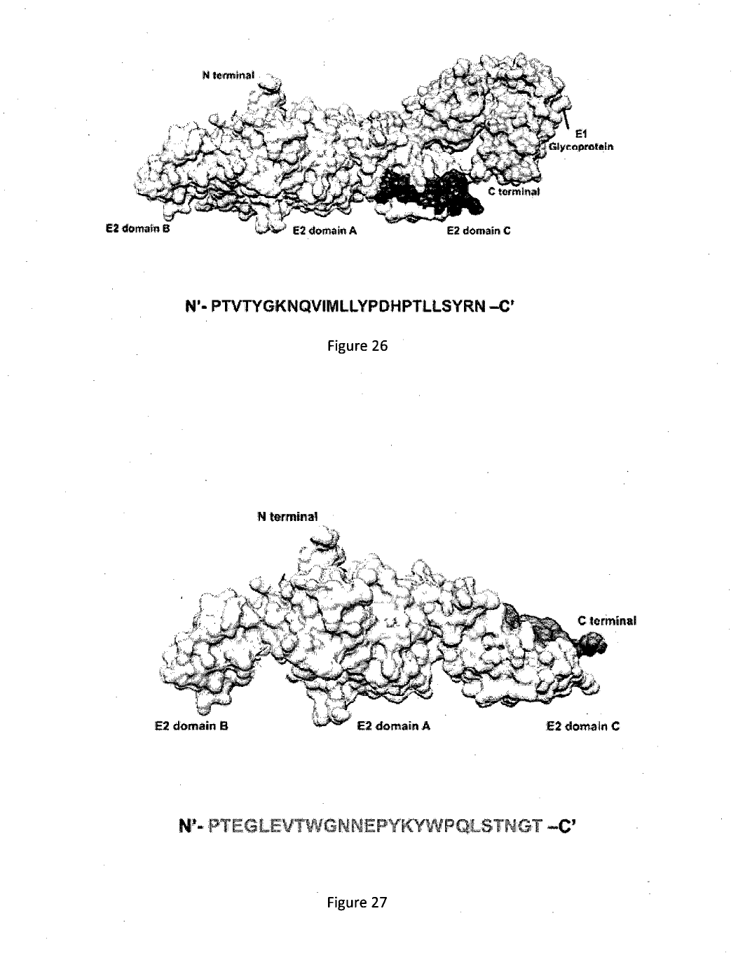

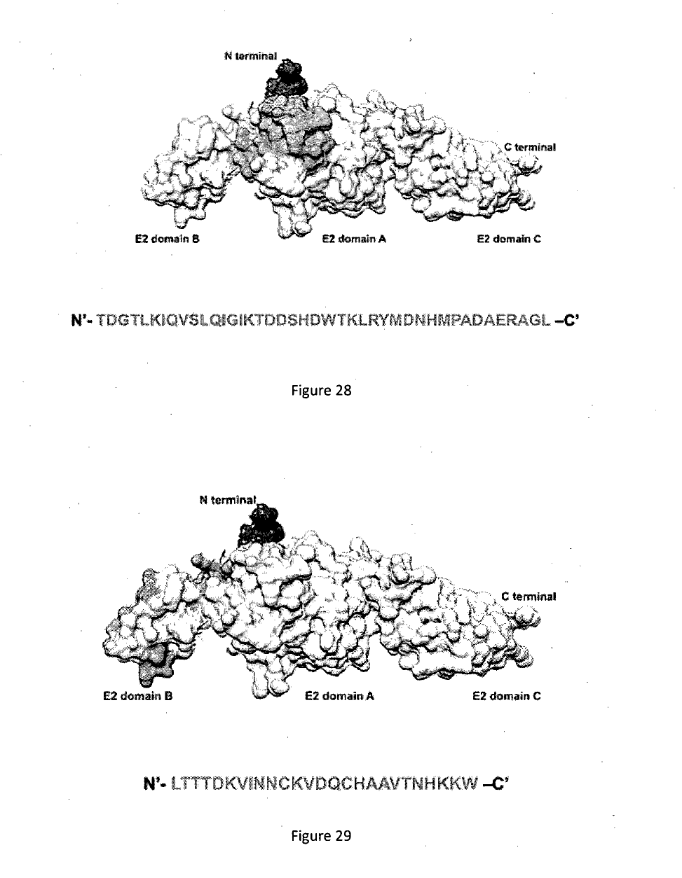

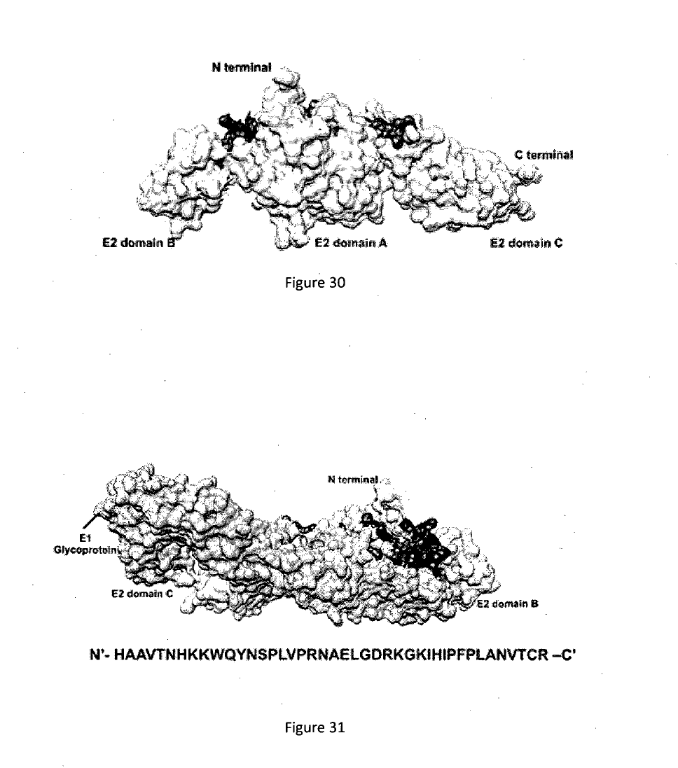

These “epitope regions” were located at the structural level of the E2 glycoprotein (FIGS. 26 to 31). The different “epitope regions” are coded for the peptides. In FIG. 26, a front view of localisation of peptides (equivalently denoted as SEQ ID Nos.) 70 to 71 is provided. In FIG. 27, a front view of localisation of peptides 76 to 77 is provided. FIG. 28 shows a front view of localisation of peptides 41 to 44. FIG. 29 shows a front view of localisation of peptides 62 to 63. FIG. 30 shows a front view of localisation of peptides 64 to 67 and FIG. 31 shows a back view of localisation of peptides 64 to 67.

Peptides 83 to 85 are amino acid residues that were not resolved in the X-ray crystal structure. All other epitope regions were along the surface of the E2 glycoprotein, indicating that these regions are accessible to the CHIKV anitbodies in terms of binding.

Epitope Regions

(a) peptides (equivalently denoted as SEQ ID Nos.) 41 to 44:

TDGTLKIQVSLQIGIKTDDSHDWTKLRYMDNHMPADAERAGL

(b) peptides (equivalently denoted as SEQ ID Nos.) 62 to 63:

LTTTDKVINNCKVDQCHAAVTNHKKW

(c) peptides (equivalently denoted as SEQ ID Nos.) 64 to 67:

HAAVTNHKKWQYNSPLVPRNAELGDRKGKIHIPFPLANVTCR

(d) peptides (equivalently denoted as SEQ ID Nos.) 70 to 71:

PTVTYGKNQVIMLLYPDHPTLLSYRN

(e) peptides (equivalently denoted as SEQ ID Nos.) 76 to 77:

PTEGLEVTWGNNEPYKYWPQLSTNGT

(f) peptides (equivalently denoted as SEQ ID Nos.) 83 to 85:

LLSMVGMAAGMCMCARRRCITPYELTPGATVPFL.

E2 proteins from the above alphaviruses were found to possess at least 70% sequence similarity to the Chikungunya E2 consensus sequence.

A first detailed longitudinal analysis of the antibody response was conducted in a cohort of patients detected early during a CHIKF outbreak. The study revealed that antibodies of the IgG3 isotype dominated the humoral response against CHIKV.

The analysis of the cohort data revealed a clear correlation between efficient viral clearance and clinical protection against persistent arthralgia and the early production of IgG3 antibodies. A putative explanation may be that late IgG3 responders established elevated levels of virus-specific IgG3 only at late phase, a time where virus was no longer detectable in the blood. In joint biopsies of patients with chronic arthralgia, CHIKV was detected in cells such as macrophages. This observation was also confirmed by studies in a non-human primate model (Labadie K, Larcher T, Joubert C, et al., “Chikungunya disease in nonhuman primates involves long-term viral persistence in macrophages”, J Clin Invest, 2010, 120, pp. 894-906).

It is plausible that the viruses in these cells are non-replicative, so that only few virions are released. These two studies may therefore propose that viral reservoirs existed in the afflicted joints, suggesting that CHIKV harboring at these sites may be protected from the neutralization action of the anti-CHIKV IgG3 antibodies. Late IgG3 responders may therefore be more prone to persistent complications.

The early increase of CHIKV IgG3 was associated with an efficient viral clearance in vivo, an effect presumably mediated by an inhibition of virus invasion and/or replication in host cells. The neutralizing effect of IgG3 antibodies was also evident in in vitro infection assays. Exposure of CHIKV to IgG3-depleted patient plasma partly prevented its inhibitory effect on the viral infection of 293T cells. While the elevated titers of early CHIKV-specific antibodies were apparently induced by high viremia, the isotype selection may be linked to IL-6. The early increase of IgG3, apparently induced by a high viremia, was clearly associated with a higher production of the cytokine, which is known to be a major B-cell growth factor and as an inducer of IgG3.

Due to the explosive nature of CHIKV outbreaks and the unpreparedness of the healthcare system in countries where they occurred, no longitudinal studies on anti-CHIV immune responses have been previously performed in this manner. It would be of interest to confirm the findings with cohorts from different parts of the world where CHIKV outbreaks have been reported. The association of anti-CHIKV IgG3 with clinical severity may allow for more cost-effective patient management since a single determination during acute phase may help predict severity.

Further, these studies viewed in the broader context of immune markers of protection against viral diseases, suggested that the production of protective IgG3 antibodies correlated with the virus titer. The paradoxical situation emerged in which a high viral load during the acute phase may be beneficial to establish full protection for the chronic phase. Low viremia, in contrast, which caused less severe symptoms during the initial phase, was often found to be associated with persistent arthralgia at later stages of the disease. Thus, the timely induction of high titers of neutralizing IgG3 may be crucial to prevent persistent complications arising from chronic viral infections. While these may have important implications for prevention and treatment of CHIKF it remained to be seen if this can also be observed for other pathogens causing severe and lasting symptoms.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

Sequence IDs

| Peptide No | Sequence | MWt |
|---|---|---|
| SEQ ID NO. 1 | PVITLYGGPKMEFIPTQT | 2505.64 |
| SEQ ID NO. 2 | PKMEFIPTQTFYNRRYQP | 2829.94 |
| SEQ ID NO. 3 | QTFYNRRYQPRPWTPRST | 2867.89 |
| SEQ ID NO. 4 | QPRPWTPRSTIQIIRPRP | 2712.86 |
| SEQ ID NO. 5 | STIQIIRPRPRPQRQAGQ | 2615.7 |
| SEQ ID NO. 6 | RPRPQRQAGQLAQLISAV | 2502.58 |
| SEQ ID NO. 7 | GQLAQLISAVNKLTMRAV | 2426.59 |
| SEQ ID NO. 8 | AVNKLTMRAVPQQKPRRN | 2620.79 |
| SEQ ID NO. 9 | AVPQQKPRRNRKNKKQKQ | 2745.9 |
| SEQ ID NO. 10 | RNRKNKKQKQKQQAPQNN | 2749.8 |
| SEQ ID NO. 11 | KQKQQAPQNNTNQKKQPP | 2618.61 |
| SEQ ID NO. 12 | NNTNQKKQPPKKKPAQKK | 2618.75 |
| SEQ ID NO. 13 | PPKKKPAQKKKKPGRRER | 2670.91 |
| SEQ ID NO. 14 | KKKKPGRRERMCMKIEND | 2761.02 |
| SEQ ID NO. 15 | ERMCMKIENDCIFEVKHE | 2767.94 |
| SEQ ID NO. 16 | NDCIFEVKHEGKVTGYAC | 2526.56 |
| SEQ ID NO. 17 | HEGKVTGYACLVGDKVMK | 2448.57 |
| SEQ ID NO. 18 | ACLVGDKVMKPAHVKGTI | 2380.58 |
| SEQ ID NO. 19 | MKPAHVKGTIDNADLAKL | 2435.55 |
| SEQ ID NO. 20 | TIDNADLAKLAFKRSSKY | 2554.61 |
| SEQ ID NO. 21 | KLAFKRSSKYDLECAQIP | 2610.74 |
| SEQ ID NO. 22 | KYDLECAQIPVHMKSDAS | 2548.6 |
| SEQ ID NO. 23 | IPVHMKSDASKFTHEKPE | 2594.66 |
| SEQ ID NO. 24 | ASKFTHEKPEGYYNWHHG | 2701.62 |
| SEQ ID NO. 25 | PEGYYNWHHGAVQYSGGR | 2591.46 |
| SEQ ID NO. 26 | HGAVQYSGGRFTIPTGAG | 2289.21 |
| SEQ ID NO. 27 | GRFTIPTGAGKPGDSGRP | 2284.24 |
| SEQ ID NO. 28 | AGKPGDSGRPIFDNKGRV | 2384.36 |
| SEQ ID NO. 29 | RPIFDNKGRVVAIVLGGA | 2395.51 |
| SEQ ID NO. 30 | RVVAIVLGGANEGARTAL | 2280.33 |
| SEQ ID NO. 31 | GANEGARTALSVVTWNKD | 2402.33 |
| SEQ ID NO. 32 | ALSVVTWNKDIVTKITPE | 2527.62 |
| SEQ ID NO. 33 | KDIVTKITPEGAEEWSLA | 2500.51 |
| SEQ ID NO. 34 | PEGAEEWSLAIPVMCLLA | 2442.56 |
| SEQ ID NO. 35 | SPHRQRRSTKDNFNVYKA | 2717.71 |
| SEQ ID NO. 36 | TKDNFNVYKATRPYLAHC | 2654.71 |
| SEQ ID NO. 37 | KATRPYLAHCPDCGEGHS | 2455.44 |
| SEQ ID NO. 38 | HCPDCGEGHSCHSPVALE | 2391.33 |
| SEQ ID NO. 39 | HSCHSPVALERIRNEATD | 2548.5 |
| SEQ ID NO. 40 | LERIRNEATDGTLKIQVS | 2556.58 |
| SEQ ID NO. 41 | TDGTLKIQVSLQIGIKTD | 2443.5 |
| SEQ ID NO. 42 | VSLQIGIKTDDSHDWTKL | 2569.58 |
| SEQ ID NO. 43 | TDDSHDWTKLRYMDNHMP | 2775.74 |
| SEQ ID NO. 44 | KLRYMDNHMPADAERAGL | 2601.67 |
| SEQ ID NO. 45 | MPADAERAGLFVRTSAPC | 2405.46 |
| SEQ ID NO. 46 | GLFVRTSAPCTITGTMGH | 2362.44 |
| SEQ ID NO. 47 | PCTITGTMGHFILARCPK | 2459.67 |
| SEQ ID NO. 48 | GHFILARCPKGETLTVGF | 2459.58 |
| SEQ ID NO. 49 | PKGETLTVGFTDSRKISH | 2486.49 |
| SEQ ID NO. 50 | GFTDSRKISHSCTHPFHH | 2607.58 |
| SEQ ID NO. 51 | SHSCTHPFHHDPPVIGRE | 2566.52 |
| SEQ ID NO. 52 | HHDPPVIGREKFHSRPQH | 2687.69 |
| SEQ ID NO. 53 | REKFHSRPQHGKELPCST | 2650.68 |
| SEQ ID NO. 54 | QHGKELPCSTYVQSTAAT | 2434.39 |
| SEQ ID NO. 55 | STYVQSTAATTEEIEVHM | 2510.44 |
| SEQ ID NO. 56 | ATTEEIEVHMPPDTPDRT | 2552.48 |
| SEQ ID NO. 57 | HMPPDTPDRTLMSQQSGN | 2525.48 |
| SEQ ID NO. 58 | RTLMSQQSGNVKITVNGQ | 2474.5 |
| SEQ ID NO. 59 | GNVKITVNGQTVRYKCNC | 2510.6 |
| SEQ ID NO. 60 | GQTVRYKCNCGGSNEGLT | 2400.36 |
| SEQ ID NO. 61 | NCGGSNEGLTTTDKVINN | 2350.23 |
| SEQ ID NO. 62 | LTTTDKVINNCKVDQCHA | 2516.56 |
| SEQ ID NO. 63 | NNCKVDQCHAAVTNHKKW | 2609.66 |
| SEQ ID NO. 64 | HAAVTNHKKWQYNSPLVP | 2603.65 |
| SEQ ID NO. 65 | KWQYNSPLVPRNAELGDR | 2656.66 |
| SEQ ID NO. 66 | VPRNAELGDRKGKIHIPF | 2560.66 |
| SEQ ID NO. 67 | DRKGKIHIPFPLANVTCR | 2578.75 |
| SEQ ID NO. 68 | PFPLANVTCRVPKARNPT | 2494.62 |
| SEQ ID NO. 69 | CRVPKARNPTVTYGKNQV | 2544.64 |
| SEQ ID NO. 70 | PTVTYGKNQVIMLLYPDH | 2602.72 |
| SEQ ID NO. 71 | QVIMLLYPDHPTLLSYRN | 2686.84 |
| SEQ ID NO. 72 | DHPTLLSYRNMGEEPNYQ | 2677.61 |
| SEQ ID NO. 73 | RNMGEEPNYQEEWVMHKK | 2818.85 |
| SEQ ID NO. 74 | YQEEWVMHKKEVVLTVPT | 2729.86 |
| SEQ ID NO. 75 | KKEVVLTVPTEGLEVTWG | 2498.58 |
| SEQ ID NO. 76 | PTEGLEVTWGNNEPYKYW | 2696.64 |
| SEQ ID NO. 77 | WGNNEPYKYWPQLSTNGT | 2668.59 |
| SEQ ID NO. 78 | YWPQLSTNGTAHGHPHEI | 2558.48 |
| SEQ ID NO. 79 | GTAHGHPHEIILYYYELY | 2689.69 |
| SEQ ID NO. 80 | EIILYYYELYPTMTVVVV | 2721.92 |
| SEQ ID NO. 81 | LYPTMTVVVVSVATFILL | 2479.73 |
| SEQ ID NO. 82 | VVSVATFILLSMVGMAAG | 2279.47 |
| SEQ ID NO. 83 | LLSMVGMAAGMCMCARRR | 2470.8 |
| SEQ ID NO. 84 | AGMCMCARRRCITPYELT | 2588.83 |
| SEQ ID NO. 85 | RRCITPYELTPGATVPFL | 2547.68 |
| SEQ ID NO. 86 | LTPGATVPFLLSLICCIR | 2430.68 |
| SEQ ID NO. 87 | FLLSLICCIRTAKAATYQ | 2528.75 |
| SEQ ID NO. 88 | IRTAKAATYQEAAIYLWN | 2596.65 |
| SEQ ID NO. 89 | STKDNFNVYKATRPYLAHC | |
| SEQ ID NO. 90 | STKDNFNVYKATRPYL | |
| SEQ ID NO. 91 | STKDHFNVYKATRPYLAHC | |
| SEQ ID NO. 92 | KXNXXVXK | |
| SEQ ID NO. 93 | KXNXXVYK | |
| SEQ ID NO. 94 | KXHXXVXK | |
| SEQ ID NO. 95 | KXHXXVYK | |
| SEQ ID NO. 96 | TDGTLKIQVSLQIGIKTDDSHDWTKLRYMDNHMPADAERAGL | |
| SEQ ID NO. 97 | LTTTDKVINNCKVDQCHAAVTNHKKW | |
| SEQ ID NO. 98 | HAAVTNHKKWQYNSPLVPRNAELGDRKGKIHIPFPLANVTCR | |
| SEQ ID NO. 99 | PTVTYGKNQVIMLLYPDHPTLLSYRN | |
| SEQ ID NO. 100 | PTEGLEVTWGNNEPYKYWPQLSTNGT | |
| SEQ ID NO. 101 | LLSMVGMAAGMCMCARRRCITPYELTPGATVPFL | |

Wherein X can be any natural occuring amino acid
Capsid Seq 1:
MEFIPTQTFYNRRYQPRPWTPRPTIQVIRPRPRPQRQAGQLAQLISAVNKLTMRAVPQQK
PRRNRKNKKQKQKQQAPQNNTNQKKQPPKKKPAQKKKKPGRRERMCMKIENDCIFEVKHE
GKVTGYACLVGDKVMKPAHVKGTIDNADLAKLAFKRSSKYDLECAQIPVHMKSDASKFTH
EKPEGYYNWHHGAVQYSGGRFTIPTGAGKPGDSGRPIFDNKGRVVAIVLGGANEGARTAL
SVVTWNKDIVTKITPEGAEEWN
Capsid Seq 2:
MEFIPTQTFYNRRYQPRPWTPRSTIQIIRPRPRPQRQAGQLAQLISAVNKLTMRAVPQQK
PRRNRKNKKQKQKQQAPQNNTNQKKQPPKKKPAQKKKKPGRRERMCMKIENDCIFEVKHE
GKVTGYACLVGDKVMKPAHVKGTIDNADLAKLAFKRSSKYDLECAQIPVHMKSDASKFTH
EKPEGYYNWHHGAVQYSGGRFTIPTGAGKPGDSGRPIFDNKGRVVAIVLGGANEGARTAL
SVVTWNKDIVTKITPEGAEEWN
E2 Glycoprotein Seq 1:
STKDNFNVYKATRPYLAHCPDCGEGHSCHSPVALERIRNEATDGTLKIQVSLQIGIKTDD
SHDWTKLRYMDNHMPADAERAGLFVRTSAPCTITGTMGHFILARCPKGETLTVGFTDSRK
ISHSCTHPFHHDPPVIGREKFHSRPQHGKELPCSTYVQSTAATTEEIEVHMPPDTPDRTL
MSQQSGNVKITVNGQTVRYKCNCGGSNEGLTTTDKVINNCKVDQCHAAVTNHKKWQYNSP
LVPRNAELGDRKGKIHIPFPLANVTCRVPKARNPTVTYGKNQVIMLLYPDHPTLLSYRNM
GEEPNYQEEWVMHKKEVVLTVPTEGLEVTWGNNEPYKYWPQLSTNGTAHGHPHEIILYYY
ELYPTMTVVVVSVATFILLSMVGMAAGMCMCARRRCITPYELTPGATVPFLLSLICCIRT
AKA
E2 Glycoprotein Seq 2:
STKDNFNVYKATRPYLAHCPDCGEGHSCHSPVALERIRNEATDGTLKIQVSLQIGIKTDD
SHDWTKLRYMDNHMPADAERAGLFVRTSAPCTITGTMGHFILARCPKGETLTVGFTDSRK
ISHSCTHPFHHDPPVIGREKFHSRPQHGKELPCSTYVQSTAATTEEIEVHMPPDTPDRTL
MSQQSGNVKITVNGQTVRYKCNCGGSNEGLTTTDKVINNCKVDQCHAAVTNHKKWQYNSP
LVPRNAELGDRQGKIHIPFPLANVTCRVPKARNPTVTYGKNQVIMLLYPDHPTLLSYRNM
GEEPNYQEEWVMHKKEVVLTVPTEGLEVTWGNNEPYKYWPQLSTNGTAHGHPHEIILYYY
ELYPTMTVVVVSVATFILLSMVGMAAGMCMCARRRCITPYELTPGATVPFLLSLICCIRT
AKA

1. Isolated immunogenic peptide,
wherein the isolated immunogenic peptide is selected from the group consisting of:
(1) peptides comprising the amino acid sequence set forth in any one of SEQ ID Nos. 1 to 95;
(2) peptides consisting of the amino acid sequence set forth in any one of SEQ ID Nos. 1 to 95;
(3) peptides comprising at least 6, 7, 8, 9 or 10 contiguous amino acids of any one of the amino acid sequences set forth in SEQ ID Nos. 96 to 101;
(4) peptides comprising an amino acid sequence that is at least 50, 60, 70, 80 or 90% identical to the sequence of any one of the peptides of (1) to (3);
(5) peptides comprising an amino acid sequence that has at least 50, 60, 70, 80 or 90% sequence similarity to the sequence of any one of the peptides of (1) to (3); and
(6) peptides according to any one of (1) to (5), wherein the peptide comprises at least one chemically modified amino acid.

2-3. (canceled)

4. The isolated immunogenic peptide as claimed in claim 1, wherein the peptide comprises a B-cell epitope that binds to a B cell receptor with detectable affinity.

5. (canceled)

6. The isolated immunogenic peptide as claimed in claim 4, wherein the dissociation constant K_{D} of the peptide for the B cell receptor is at least about 10⁻⁶ M.

7. The isolated immunogenic peptide as claimed in claim 1, wherein the peptide is capable of eliciting an IgG or IgM antibody response in a human subject.

8. The isolated immunogenic peptide as claimed in claim 7, wherein the IgG antibody response is an IgG3 antibody response.

9. The peptide as claimed in claim 1, wherein the peptide is coupled to a detectable label.

10. The isolated immunogenic peptide as claimed in claim 9, wherein the label is selected from the group consisting of a fluorophor, a chromophor, a radiolabel, biotin, streptavidin, a Strep-tag, a 6×His-tag, a Myc-tag, and an enzyme.

11. The isolated immunogenic peptide as claimed in claim 1 encoded by a nucleic acid molecule.

12. The isolated immunogenic peptide as claimed in claim 11 wherein the nucleic acid molecule is comprised in a Vector.

13. (canceled)

14. The isolated immunogenic peptide as claimed in claim 11 or 12 wherein the nucleic acid molecule expresses the peptide in a Recombinant cell.

15-16. (canceled)

17. The isolated immunogenic peptide as claimed in claim 11 or 12 wherein the nucleic acid molecule expresses the peptide in a recombinant cell, wherein the cell is a dendritic cell, monocyte or B lymphocyte.

18. (canceled)

19. Antibody specifically binding the isolated immunogenic peptide as claimed in claim 1.

20. The antibody as claimed in claim 19, wherein the antibody binds the peptide with a dissociation constant (K_{D}) of at least 10⁻⁶ M.

21. The isolated immunogenic peptide as claimed in claim 1 further comprising one or more isolated immunogenic peptides and a pharmaceutically acceptable carrier and/or pharmaceutically acceptable excipients.

22. (canceled)

23. The isolated immunogenic peptide as claimed in claim 1, further comprising at least one immunostimulatory agent comprising a adjuvant or a cytokine selected from the group consisting of complete and incomplete Freud's adjuvant, tripalmitoyl-S-glyceryl-cystein, aluminium salts, virosomes, squalene, MF59, monophosphoryl lipid A, QS21, CpG motifs, ISCOMS (structured complex of saponins and lipids), or Advax.

24-25. (canceled)

26. The isolated immunogenic peptide as claimed in claim 1, wherein the isolated immunogenic peptide is bound to an antigen-presenting cell (APC).

27. Method for vaccinating a subject against Alphaviruses, comprising administering to said subject a therapeutically effective amount of an isolated immunogenic peptide as claimed in claim 1, 14 or 16.

28-29. (canceled)

30. Method for monitoring an Alphavirus infection in a subject, comprising contacting a sample obtained from said subject with one or more isolated immunogenic peptides as claimed in claim 1 and determining the level of antibodies specifically binding to said one or more peptides.

31-33. (canceled)

34. The method as claimed in claim 30, wherein the Alphavirus is selected from the group consisting of Chikungunya Virus (CHIKV), Sindbis Virus, Semliki Forest Virus, Mayaro Virus, Ross River Virus, Barmah Forest Virus, Eastern Equine Encephalitis Virus, Western Equine Encephalitis Virus, O'Nyong Nyong Virus (ONNV), Venezuelan Equine Encephalitis Virus, Aura Virus, Bebaru Virus, Cabassou Virus, Eastern Everglades Virus, Fort Morgan Virus, Getah Virus, Highlands J Virus, Middelburg Virus; Mosso das Pedras Virus (78V3531), Mucambo Virus, Ndumu Virus, Pixuna Virus, Rio Negro Virus, Salmon Pancreas Disease Virus, Southern Elephant Seal Virus, Tonate Virus, Trocara Virus, Una Virus, and Whataroa Virus.

35. (canceled)

36. Method as claimed in claim 30, further comprising determining the level of neutralizing IgG3 antibodies specific for a CHIKV antigen in a sample obtained from said patient by contacting said sample with said isolated immunogenic peptides to form a peptide:antibody complexe and detecting the presence and amount of said complexe, wherein antibody levels in a post-acute phase that are higher than amount of a healthy control or a mean value obtained from the healthy control±3SD are indicative of a lower risk for persistent arthralgia and/or the development of full protective immunity.

37. (canceled)

38. The method as claimed in claim 36, wherein the CHIKV antigen is a CHIKV E2 glycoprotein antigen.

39-42. (canceled)


SEQUENCE LISTING (105 sequences)

SEQ ID NO: 1 - protein, 18 aa, Artificial Sequence
  isolated immunogenic peptide
PVITLYGGPKMEFIPTQT

SEQ ID NO: 2 - protein, 18 aa, Artificial Sequence
  isolated immunogenic peptide
PKMEFIPTQTFYNRRYQP

SEQ ID NO: 3 - protein, 18 aa, Artificial Sequence
  isolated immunogenic peptide
QTFYNRRYQPRPWTPRST

SEQ ID NO: 4 - protein, 18 aa, Artificial Sequence
  isolated immunogenic peptide
QPRPWTPRSTIQIIRPRP

SEQ ID NO: 5 - protein, 18 aa, Artificial Sequence
  isolated immunogenic peptide
STIQIIRPRPRPQRQAGQ

SEQ ID NO: 6 - protein, 18 aa, Artificial Sequence
  isolated immunogenic peptide
RPRPQRQAGQLAQLISAV

SEQ ID NO: 7 - protein, 18 aa, Artificial Sequence
  isolated immunogenic peptide
GQLAQLISAVNKLTMRAV

SEQ ID NO: 8 - protein, 18 aa, Artificial Sequence
  isolated immunogenic peptide
AVNKLTMRAVPQQKPRRN

SEQ ID NO: 9 - protein, 18 aa, Artificial Sequence
  isolated immunogenic peptide
AVPQQKPRRNRKNKKQKQ

SEQ ID NO: 10 - protein, 18 aa, Artificial Sequence
  isolated immunogenic peptide
RNRKNKKQKQKQQAPQNN

SEQ ID NO: 11 - protein, 18 aa, Artificial Sequence
  isolated immunogenic peptide
KQKQQAPQNNTNQKKQPP

SEQ ID NO: 12 - protein, 18 aa, Artificial Sequence
  isolated immunogenic peptide
NNTNQKKQPPKKKPAQKK

SEQ ID NO: 13 - protein, 18 aa, Artificial Sequence
  isolated immunogenic peptide
PPKKKPAQKKKKPGRRER

SEQ ID NO: 14 - protein, 18 aa, Artificial Sequence
  isolated immunogenic peptide
KKKKPGRRERMCMKIEND

SEQ ID NO: 15 - protein, 18 aa, Artificial Sequence
  isolated immunogenic peptide
ERMCMKIENDCIFEVKHE

SEQ ID NO: 16 - protein, 18 aa, Artificial Sequence
  isolated immunogenic peptide
NDCIFEVKHEGKVTGYAC

SEQ ID NO: 17 - protein, 18 aa, Artificial Sequence
  isolated immunogenic peptide
HEGKVTGYACLVGDKVMK

SEQ ID NO: 18 - protein, 18 aa, Artificial Sequence
  isolated immunogenic peptide
ACLVGDKVMKPAHVKGTI

SEQ ID NO: 19 - protein, 18 aa, Artificial Sequence
  isolated immunogenic peptide
MKPAHVKGTIDNADLAKL

SEQ ID NO: 20 - protein, 18 aa, Artificial Sequence
  isolated immunogenic peptide
TIDNADLAKLAFKRSSKY

SEQ ID NO: 21 - protein, 18 aa, Artificial Sequence
  isolated immunogenic peptide
KLAFKRSSKYDLECAQIP

SEQ ID NO: 22 - protein, 18 aa, Artificial Sequence
  isolated immunogenic peptide
KYDLECAQIPVHMKSDAS

SEQ ID NO: 23 - protein, 18 aa, Artificial Sequence
  isolated immunogenic peptide
IPVHMKSDASKFTHEKPE

SEQ ID NO: 24 - protein, 18 aa, Artificial Sequence
  isolated immunogenic peptide
ASKFTHEKPEGYYNWHHG

SEQ ID NO: 25 - protein, 18 aa, Artificial Sequence
  isolated immunogenic peptide
PEGYYNWHHGAVQYSGGR

SEQ ID NO: 26 - protein, 18 aa, Artificial Sequence
  isolated immunogenic peptide
HGAVQYSGGRFTIPTGAG

SEQ ID NO: 27 - protein, 18 aa, Artificial Sequence
  isolated immunogenic peptide
GRFTIPTGAGKPGDSGRP

SEQ ID NO: 28 - protein, 18 aa, Artificial Sequence
  isolated immunogenic peptide
AGKPGDSGRPIFDNKGRV

SEQ ID NO: 29 - protein, 18 aa, Artificial Sequence
  isolated immunogenic peptide
RPIFDNKGRVVAIVLGGA

SEQ ID NO: 30 - protein, 18 aa, Artificial Sequence
  isolated immunogenic peptide
RVVAIVLGGANEGARTAL

SEQ ID NO: 31 - protein, 18 aa, Artificial Sequence
  isolated immunogenic peptide
GANEGARTALSVVTWNKD

SEQ ID NO: 32 - protein, 18 aa, Artificial Sequence
  isolated immunogenic peptide
ALSVVTWNKDIVTKITPE

SEQ ID NO: 33 - protein, 18 aa, Artificial Sequence
  isolated immunogenic peptide
KDIVTKITPEGAEEWSLA

SEQ ID NO: 34 - protein, 18 aa, Artificial Sequence
  isolated immunogenic peptide
PEGAEEWSLAIPVMCLLA

SEQ ID NO: 35 - protein, 18 aa, Artificial Sequence
  isolated immunogenic peptide
SPHRQRRSTKDNFNVYKA

SEQ ID NO: 36 - protein, 18 aa, Artificial Sequence
  isolated immunogenic peptide
TKDNFNVYKATRPYLAHC

SEQ ID NO: 37 - protein, 18 aa, Artificial Sequence
  isolated immunogenic peptide
KATRPYLAHCPDCGEGHS

SEQ ID NO: 38 - protein, 18 aa, Artificial Sequence
  isolated immunogenic peptide
HCPDCGEGHSCHSPVALE

SEQ ID NO: 39 - protein, 18 aa, Artificial Sequence
  isolated immunogenic peptide
HSCHSPVALERIRNEATD

SEQ ID NO: 40 - protein, 18 aa, Artificial Sequence
  isolated immunogenic peptide
LERIRNEATDGTLKIQVS

SEQ ID NO: 41 - protein, 18 aa, Artificial Sequence
  isolated immunogenic peptide
TDGTLKIQVSLQIGIKTD

SEQ ID NO: 42 - protein, 18 aa, Artificial Sequence
  isolated immunogenic peptide
VSLQIGIKTDDSHDWTKL

SEQ ID NO: 43 - protein, 18 aa, Artificial Sequence
  isolated immunogenic peptide
TDDSHDWTKLRYMDNHMP

SEQ ID NO: 44 - protein, 18 aa, Artificial Sequence
  isolated immunogenic peptide
KLRYMDNHMPADAERAGL

SEQ ID NO: 45 - protein, 18 aa, Artificial Sequence
  isolated immunogenic peptide
MPADAERAGLFVRTSAPC

SEQ ID NO: 46 - protein, 18 aa, Artificial Sequence
  isolated immunogenic peptide
GLFVRTSAPCTITGTMGH

SEQ ID NO: 47 - protein, 18 aa, Artificial Sequence
  isolated immunogenic peptide
PCTITGTMGHFILARCPK

SEQ ID NO: 48 - protein, 18 aa, Artificial Sequence
  isolated immunogenic peptide
GHFILARCPKGETLTVGF

SEQ ID NO: 49 - protein, 18 aa, Artificial Sequence
  isolated immunogenic peptide
PKGETLTVGFTDSRKISH

SEQ ID NO: 50 - protein, 18 aa, Artificial Sequence
  isolated immunogenic peptide
GFTDSRKISHSCTHPFHH

SEQ ID NO: 51 - protein, 18 aa, Artificial Sequence
  isolated immunogenic peptide
SHSCTHPFHHDPPVIGRE

SEQ ID NO: 52 - protein, 18 aa, Artificial Sequence
  isolated immunogenic peptide
HHDPPVIGREKFHSRPQH

SEQ ID NO: 53 - protein, 18 aa, Artificial Sequence
  isolated immunogenic peptide
REKFHSRPQHGKELPCST

SEQ ID NO: 54 - protein, 18 aa, Artificial Sequence
  isolated immunogenic peptide
QHGKELPCSTYVQSTAAT

SEQ ID NO: 55 - protein, 18 aa, Artificial Sequence
  isolated immunogenic peptide
STYVQSTAATTEEIEVHM

SEQ ID NO: 56 - protein, 18 aa, Artificial Sequence
  isolated immunogenic peptide
ATTEEIEVHMPPDTPDRT

SEQ ID NO: 57 - protein, 18 aa, Artificial Sequence
  isolated immunogenic peptide
HMPPDTPDRTLMSQQSGN

SEQ ID NO: 58 - protein, 18 aa, Artificial Sequence
  isolated immunogenic peptide
RTLMSQQSGNVKITVNGQ

SEQ ID NO: 59 - protein, 18 aa, Artificial Sequence
  isolated immunogenic peptide
GNVKITVNGQTVRYKCNC

SEQ ID NO: 60 - protein, 18 aa, Artificial Sequence
  isolated immunogenic peptide
GQTVRYKCNCGGSNEGLT

SEQ ID NO: 61 - protein, 18 aa, Artificial Sequence
  isolated immunogenic peptide
NCGGSNEGLTTTDKVINN

SEQ ID NO: 62 - protein, 18 aa, Artificial Sequence
  isolated immunogenic peptide
LTTTDKVINNCKVDQCHA

SEQ ID NO: 63 - protein, 18 aa, Artificial Sequence
  isolated immunogenic peptide
NNCKVDQCHAAVTNHKKW

SEQ ID NO: 64 - protein, 18 aa, Artificial Sequence
  isolated immunogenic peptide
HAAVTNHKKWQYNSPLVP

SEQ ID NO: 65 - protein, 18 aa, Artificial Sequence
  isolated immunogenic peptide
KWQYNSPLVPRNAELGDR

SEQ ID NO: 66 - protein, 18 aa, Artificial Sequence
  isolated immunogenic peptide
VPRNAELGDRKGKIHIPF

SEQ ID NO: 67 - protein, 18 aa, Artificial Sequence
  isolated immunogenic peptide
DRKGKIHIPFPLANVTCR

SEQ ID NO: 68 - protein, 18 aa, Artificial Sequence
  isolated immunogenic peptide
PFPLANVTCRVPKARNPT

SEQ ID NO: 69 - protein, 18 aa, Artificial Sequence
  isolated immunogenic peptide
CRVPKARNPTVTYGKNQV

SEQ ID NO: 70 - protein, 18 aa, Artificial Sequence
  isolated immunogenic peptide
PTVTYGKNQVIMLLYPDH

SEQ ID NO: 71 - protein, 18 aa, Artificial Sequence
  isolated immunogenic peptide
QVIMLLYPDHPTLLSYRN

SEQ ID NO: 72 - protein, 18 aa, Artificial Sequence
  isolated immunogenic peptide
DHPTLLSYRNMGEEPNYQ

SEQ ID NO: 73 - protein, 18 aa, Artificial Sequence
  isolated immunogenic peptide
RNMGEEPNYQEEWVMHKK

SEQ ID NO: 74 - protein, 18 aa, Artificial Sequence
  isolated immunogenic peptide
YQEEWVMHKKEVVLTVPT

SEQ ID NO: 75 - protein, 18 aa, Artificial Sequence
  isolated immunogenic peptide
KKEVVLTVPTEGLEVTWG

SEQ ID NO: 76 - protein, 18 aa, Artificial Sequence
  isolated immunogenic peptide
PTEGLEVTWGNNEPYKYW

SEQ ID NO: 77 - protein, 18 aa, Artificial Sequence
  isolated immunogenic peptide
WGNNEPYKYWPQLSTNGT

SEQ ID NO: 78 - protein, 18 aa, Artificial Sequence
  isolated immunogenic peptide
YWPQLSTNGTAHGHPHEI

SEQ ID NO: 79 - protein, 18 aa, Artificial Sequence
  isolated immunogenic peptide
GTAHGHPHEIILYYYELY

SEQ ID NO: 80 - protein, 18 aa, Artificial Sequence
  isolated immunogenic peptide
EIILYYYELYPTMTVVVV

SEQ ID NO: 81 - protein, 18 aa, Artificial Sequence
  isolated immunogenic peptide
LYPTMTVVVVSVATFILL

SEQ ID NO: 82 - protein, 18 aa, Artificial Sequence
  isolated immunogenic peptide
VVSVATFILLSMVGMAAG

SEQ ID NO: 83 - protein, 18 aa, Artificial Sequence
  isolated immunogenic peptide
LLSMVGMAAGMCMCARRR

SEQ ID NO: 84 - protein, 18 aa, Artificial Sequence
  isolated immunogenic peptide
AGMCMCARRRCITPYELT

SEQ ID NO: 85 - protein, 18 aa, Artificial Sequence
  isolated immunogenic peptide
RRCITPYELTPGATVPFL

SEQ ID NO: 86 - protein, 18 aa, Artificial Sequence
  isolated immunogenic peptide
LTPGATVPFLLSLICCIR

SEQ ID NO: 87 - protein, 18 aa, Artificial Sequence
  isolated immunogenic peptide
FLLSLICCIRTAKAATYQ

SEQ ID NO: 88 - protein, 18 aa, Artificial Sequence
  isolated immunogenic peptide
IRTAKAATYQEAAIYLWN

SEQ ID NO: 89 - protein, 19 aa, Artificial Sequence
  isolated immunogenic peptide
STKDNFNVYKATRPYLAHC

SEQ ID NO: 90 - protein, 16 aa, Artificial Sequence
  isolated immunogenic peptide
STKDNFNVYKATRPYL

SEQ ID NO: 91 - protein, 19 aa, Artificial Sequence
  isolated immunogenic peptide
STKDHFNVYKATRPYLAHC

SEQ ID NO: 92 - protein, 8 aa, Artificial Sequence
  isolated immunogenic peptide
KXNXXVXK

SEQ ID NO: 93 - protein, 8 aa, Artificial Sequence
  isolated immunogenic peptide
KXNXXVYK

SEQ ID NO: 94 - protein, 8 aa, Artificial Sequence
  isolated immunogenic peptide
KXHXXVXK

SEQ ID NO: 95 - protein, 8 aa, Artificial Sequence
  isolated immunogenic peptide
KXHXXVYK

SEQ ID NO: 96 - protein, 42 aa, Artificial Sequence
  isolated immunogenic peptide
TDGTLKIQVSLQIGIKTDDSHDWTKLRYMDNHMPADAERAGL

SEQ ID NO: 97 - protein, 26 aa, Artificial Sequence
  isolated immunogenic peptide
LTTTDKVINNCKVDQCHAAVTNHKKW

SEQ ID NO: 98 - protein, 42 aa, Artificial Sequence
  isolated immunogenic peptide
HAAVTNHKKWQYNSPLVPRNAELGDRKGKIHIPFPLANVTCR

SEQ ID NO: 99 - protein, 26 aa, Artificial Sequence
  isolated immunogenic peptide
PTVTYGKNQVIMLLYPDHPTLLSYRN

SEQ ID NO: 100 - protein, 26 aa, Artificial Sequence
  isolated immunogenic peptide
PTEGLEVTWGNNEPYKYWPQLSTNGT

SEQ ID NO: 101 - protein, 34 aa, Artificial Sequence
  isolated immunogenic peptide
LLSMVGMAAGMCMCARRRCITPYELTPGATVPFL

SEQ ID NO: 102 - protein, 262 aa, Chikungunya virus
  CAPSID SEQ 1
MEFIPTQTFYNRRYQPRPWTPRPTIQVIRPRPRPQRQAGQLAQLISAVNKLTMRAVPQQKPRRNRKNKKQKQKQQAPQNNTNQKKQPPKKKPAQKKKKPGRRERMCMKIENDCIFEVKHEGKVTGYACLVGDKVMKPAHVKGTIDNADLAKLAFKRSSKYDLECAQIPVHMKSDASKFTHEKPEGYYNWHHGAVQYSGGRFTIPTGAGKPGDSGRPIFDNKGRVVAIVLGGANEGARTALSVVTWNKDIVTKITPEGAEEWN

SEQ ID NO: 103 - protein, 262 aa, Chikungunya virus
  CAPSID SEQ 2
MEFIPTQTFYNRRYQPRPWTPRSTIQIIRPRPRPQRQAGQLAQLISAVNKLTMRAVPQQKPRRNRKNKKQKQKQQAPQNNTNQKKQPPKKKPAQKKKKPGRRERMCMKIENDCIFEVKHEGKVTGYACLVGDKVMKPAHVKGTIDNADLAKLAFKRSSKYDLECAQIPVHMKSDASKFTHEKPEGYYNWHHGAVQYSGGRFTIPTGAGKPGDSGRPIFDNKGRVVAIVLGGANEGARTALSVVTWNKDIVTKITPEGAEEWN

SEQ ID NO: 104 - protein, 423 aa, Chikungunya virus
  E2 Glycoprotein Seq 1
STKDNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSNEGLTTTDKVINNCKVDQCHAAVTNHKKWQYNSPLVPRNAELGDRKGKIHIPFPLANVTCRVPKARNPTVTYGKNQVIMLLYPDHPTLLSYRNMGEEPNYQEEWVMHKKEVVLTVPTEGLEVTWGNNEPYKYWPQLSTNGTAHGHPHEIILYYYELYPTMTVVVVSVATFILLSMVGMAAGMCMCARRRCITPYELTPGATVPFLLSLICCIRTAKA

SEQ ID NO: 105 - protein, 423 aa, Chikungunya virus
  E2 Glycoprotein Seq 2
STKDNFNVYKATRPYLAHCPDCGEGHSCHSPVALERIRNEATDGTLKIQVSLQIGIKTDDSHDWTKLRYMDNHMPADAERAGLFVRTSAPCTITGTMGHFILARCPKGETLTVGFTDSRKISHSCTHPFHHDPPVIGREKFHSRPQHGKELPCSTYVQSTAATTEEIEVHMPPDTPDRTLMSQQSGNVKITVNGQTVRYKCNCGGSNEGLTTTDKVINNCKVDQCHAAVTNHKKWQYNSPLVPRNAELGDRQGKIHIPFPLANVTCRVPKARNPTVTYGKNQVIMLLYPDHPTLLSYRNMGEEPNYQEEWVMHKKEVVLTVPTEGLEVTWGNNEPYKYWPQLSTNGTAHGHPHEIILYYYELYPTMTVVVVSVATFILLSMVGMAAGMCMCARRRCITPYELTPGATVPFLLSLICCIRTAKA